SEQUENCE LISTING (74 sequences)

SEQ ID NO: 1 - DNA, 1238 bp, Homo sapiens
cggcggagctggtcccgttgtgctgcggcgccgcgcggcctgcagtcccgggcccgcgccccgcgccgcccgcccgcccgccatggagcccggccccgacggccccgccgcctccggccccgccgccatccgcgagggctggttccgcgagacctgcagcctgtggcccggccaggccctgtcgctgcaggtggagcagctgctccaccaccggcgctcgcgctaccaggacatcctcgtcttccgcagtaagacctatggcaacgtgctggtgttggacggtgtcatccagtgcacggagagagacgagttctcctaccaggagatgatcgccaacctgcctctctgcagccaccccaacccgcgaaaggtgctgatcatcgggggcggagatggaggtgtcctgcgggaggtggtgaagcacccctccgtggagtccgtggtccagtgtgagatcgacgaggatgtcatccaagtctccaagaagttcctgccaggcatggccattggctactctagctcgaaggtgaccctacatgtgggtgacggttttgagttcatgaaacagaatcaggatgccttcgacgtgatcatcactgactcctcagaccccatgggccccgccgaaagtctcttcaaggagtcctattaccagctcatgaagacagccctcaaggaagatggtgtcctctgctgccagggcgagtgcgagtggctgcacctggacctcatcaaggagatgcggcagttctgccagtccctgttccccgtggtggcctatgcctactgcaccatccccacctaccccagcggccagatcggcttcatgctgtgcagcaagaacccgagcacgaacttccaggagccggtgcagccgctgacacagcagcaggtggcgcagatgcagctgaagtactacaactccgacgtgcaccgcgccgcctttgtgctgcccgagtttgcccgcaaggccctgaatgatgtgagctgagcccaggcgccaccactgatgccacccaggacctcggaccttggagcctgcggggtgcctcggcccctccagccccgggccggacctcctgctggctctcgcccaccaaccaagtgttacaagccccagaatgctgcccggcctgccctgctgggcggactgtctgtgtgtctgtctctctggcgttccacctccaagcctataccagctgtgtacagcgccatctctctgccttctgttgcccccg

SEQ ID NO: 2 - protein, 302 aa, Homo sapiens
MEPGPDGPAASGPAAIREGWFRETCSLWPGQALSLQVEQLLHHRRSRYQDILVFRSKTYGNVLVLDGVIQCTERDEFSYQEMIANLPLCSHPNPRKVLIIGGGDGGVLREVVKHPSVESVVQCEIDEDVIQVSKKFLPGMAIGYSSSKVTLHVGDGFEFMKQNQDAFDVIITDSSDPMGPAESLFKESYYQLMKTALKEDGVLCCQGECEWLHLDLIKEMRQFCQSLFPVVAYAYCTIPTYPSGQIGFMLCSKNPSTNFQEPVQPLTQQQVAQMQLKYYNSDVHRAAFVLPEFARKALNDVS

SEQ ID NO: 3 - protein, 520 aa, Drosophila
MMAASGRIRKRKHKSHTSGDVPSTTTSVPMPIPTMAPGKMVAETMEEAAALAGDYNNFTHNFVDLQNLLSFNELNGTSGSGGTAVSSLGSSSAIKLNNSAITDTLLGTVLTTATATVAPAASSLLATLAATTTASARGSLAGKSLAIADATSSTYYSNLLNLSPATTSLISAAAATKSYNDSALRWEQLDGSVDFGFDPLYRHSLAMSMVYCVAYIVVFLVGLIGNSFVIAVVLRAPRMRTVTNYFIVNLAIADILVIVFCLPATLIGNTWMLGWLMCKFVPYIQGVSVAASVYSLIAVSLDRFIAIWWPLKQMTKRRARIMIIGIWVIALVTTIPWLLFFDLVPAEEVFSDALVSAYSQPQFLCQEVWPPGTDGNLYFLLANLVACYLLPMSLITLCYVLIWIKVSTRSIPGESKDAQMDRMQQKSKVKVIKMLVAVVILFVLSWLPLYVIFARIKFGSDISQEEFEILKKVMPVAQWLGSSNSCINPILYSVNKKYRRGFAAIIKSRSCCGRLRLVNF

SEQ ID NO: 4 - DNA, 1560 bp, Homo sapiens
tgcctctgcccacctcttctcttctgcttccatattacaggttcatcatgaatgagaaatgggacacaaactcttcagaaaactggcatcccatctggaatgtcaatgacacaaagcatcatctgtactcagatattaatattacctatgtgaactactatcttcaccagcctcaagtggcagcaatcttcattatttcctactttctgatcttctttttgtgcatgatgggaaatactgtggtttgctttattgtaatgaggaacaaacatatgcacacagtcactaatctcttcatcttaaacctggccataagtgatttactagttggcatattctgcatgcctataacactgctggacaatattatagcaggatggccatttggaaacacgatgtgcaagatcagtggattggtccagggaatatctgtcgcagcttcagtctttacgttagttgcaattgctgtagataggttccagtgtgtggtctacccttttaaaccaaagctcactatcaagacagcgtttgtcattattatgatcatctgggtcctagccatcaccattatgtctccatctgcagtaatgttacatgtgcaagaagaaaaatattaccgagtgagactcaactcccagaataaaaccagtccagtctactggtgccgggaagactggccaaatcaggaaatgaggaagatctacaccactgtgctgtttgccaacatctacctggctcccctctccctcattgtcatcatgtatggaaggattggaatttcactcttcagggctgcagttcctcacacaggcaggaagaaccaggagcagtggcacgtggtgtccaggaagaagcagaagatcattaagatgctcctgattgtggccctgctttttattctctcatggctgcccctgtggactctaatgatgctctcagactacgctgacctttctccaaatgaactgcagatcatcaacatctacatctacccttttgcacactggctggcattcggcaacagcagtgtcaatcccatcatttatggtttcttcaacgagaatttccgccgtggtttccaagaagctttccagctccagctctgccaaaaaagagcaaagcctatggaagcttatgccctaaaagctaaaagccatgtgctcataaacacatctaatcagcttgtccaggaatctacatttcaaaaccctcatggggaaaccttgctttataggaaaagtgctgaaaaaccccaacaggaattagtgatggaagaattaaaagaaactactaacagcagtgagatttaaaaagagctagtgtgataatcctaactctactacgcattatatatttaaatccattgctttttgtggctttgcacttcaaatttttcaaagaatgttctaaataaaacatttactgaaagccctctctggcaaaaaaattaaaaataaacaaaaatggtcataagatcataaacaatcttatgttgtataaaaatacgtagagtgacttagacatgtttgcatgaataaatatatttctagagaacagtta

SEQ ID NO: 5 - protein, 420 aa, Homo sapiens
MNEKWDTNSSENWHPIWNVNDTKHHLYSDINITYVNYYLHQPQVAAIFIISYFLIFFLCMMGNTVVCFIVMRNKHMHTVTNLFILNLAISDLLVGIFCMPITLLDNIIAGWPFGNTMCKISGLVQGISVAASVFTLVAIAVDRFQCVVYPFKPKLTIKTAFVIIMIIWVLAITIMSPSAVMLHVQEEKYYRVRLNSQNKTSPVYWCREDWPNQEMRKIYTTVLFANIYLAPLSLIVIMYGRIGISLFRAAVPHTGRKNQEQWHVVSRKKQKIIKMLLIVALLFILSWLPLWTLMMLSDYADLSPNELQIINIYIYPFAHWLAFGNSSVNPIIYGFFNENFRRGFQEAFQLQLCQKRAKPMEAYALKAKSHVLINTSNQLVQESTFQNPHGETLLYRKSAEKPQQELVMEELKETTNSSEI

SEQ ID NO: 6 - DNA, 1293 bp, Homo sapiens
atggagggggagccctcccagcctcccaacagcagttggcccctaagtcagaatgggactaacactgaggccaccccggctacaaacctcaccttctcctcctactatcagcacacctcccctgtggcggccatgttcattgtggcctatgcgctcatcttcctgctctgcatggtgggcaacaccctggtctgtttcatcgtgctcaagaaccggcacatgcatactgtcaccaacatgttcatcctcaacctggctgtcagtgacctgctggtgggcatcttctgcatgcccaccacccttgtggacaacctcatcactgggtggcccttcgacaatgccacatgcaagatgagcggcttggtgcagggcatgtctgtgtcggcttccgttttcacactggtggccattgctgtggaaaggttccgctgcatcgtgcaccctttccgcgagaagctgaccctgcggaaggcgctcgtcaccatcgccgtcatctgggccctggcgctgctcatcatgtgtccctcggccgtcacgctgaccgtcacccgtgaggagcaccacttcatggtggacgcccgcaaccgctcctaccctctctactcctgctgggaggcctggcccgagaagggcatgcgcagggtctacaccactgtgctcttctcgcacatctacctggcgccgctggcgctcatcgtggtcatgtacgcccgcatcgcgcgcaagctctgccaggccccgggcccggcccccgggggcgaggaggctgcggacccgcgagcatcgcggcgcagagcgcgcgtggtgcacatgctggtcatggtggcgctgttcttcacgctgtcctggctgccgctctgggcgctgctgctgctcatcgactacgggcagctcagcgcgccgcagctgcacctggtcaccgtctacgccttccccttcgcgcactggctggccttcttcaacagcagcgccaaccccatcatctacggctacttcaacgagaacttccgccgcggcttccaggccgccttccgcgcccgcctctgcccgcgcccgtcggggagccacaaggaggcctactccgagcggcccggcgggcttctgcacaggcgggtcttcgtggtggtgcggcccagcgactccgggctgccctctgagtcgggccctagcagtggggcccccaggcccggccgcctcccgctgcggaatgggcgggtggctcaccacggcttgcccagggaagggcctggctgctcccacctgcccctcaccattccagcctgggatatctga

SEQ ID NO: 7 - protein, 430 aa, Homo sapiens
MEGEPSQPPNSSWPLSQNGTNTEATPATNLTFSSYYQHTSPVAAMFIVAYALIFLLCMVGNTLVCFIVLKNRHMHTVTNMFILNLAVSDLLVGIFCMPTTLVDNLITGWPFDNATCKMSGLVQGMSVSASVFTLVAIAVERFRCIVHPFREKLTLRKALVTIAVIWALALLIMCPSAVTLTVTREEHHFMVDARNRSYPLYSCWEAWPEKGMRRVYTTVLFSHIYLAPLALIVVMYARIARKLCQAPGPAPGGEEAADPRASRRRARVVHMLVMVALFFTLSWLPLWALLLLIDYGQLSAPQLHLVTVYAFPFAHWLAFFNSSANPIIYGYFNENFRRGFQAAFRARLCPRPSGSHKEAYSERPGGLLHRRVFVVVRPSDSGLPSESGPSSGAPRPGRLPLRNGRVAHHGLPREGPGCSHLPLTIPAWDI

SEQ ID NO: 8 - DNA, 1564 bp, Homo sapiens
cctcccttcaggaagtttgaggctgagacccgaaaagacctgggtgcaagcctccaggcaccctgaagggagtgggctgagggctggcccaagctccctcctctccctctgtagagcctaggatgcccctctgctgcagcggctcctgagctcatggagccctcagccaccccaggggcccagatgggggtcccccctggcagcagagagccgtcccctgtgcctccagactatgaagatgagtttctccgctatctgtggcgtgattatctgtacccaaaacagtatgagtgggtcctcatcgcagcctatgtggctgtgttcgtcgtggccctggtgggcaacacgctggtctgcctggccgtgtggcggaaccaccacatgaggacagtcaccaactacttcattgtcaacctgtccctggctgacgttctggtgactgctatctgcctgccggccagcctgctggtggacatcactgagtcctggctgttcggccatgccctctgcaaggtcatcccctatctacaggctgtgtccgtgtcagtggcagtgctaactctcagcttcatcgccctggaccgctggtatgccatctgccacccactattgttcaagagcacagcccggcgggcccgtggctccatcctgggcatctgggctgtgtcgctggccatcatggtgccccaggctgcagtcatggaatgcagcagtgtgctgcctgagctagccaaccgcacacggctcttctcagtctgtgatgaacgctgggcagatgacctctatcccaagatctaccacagttgcttctttattgtcacctacctggccccactgggcctcatggccatggcctatttccagatattccgcaagctctggggccgccagatccccggcaccacctcagcactggtgcggaactggaagcgcccctcagaccagctgggggacctggagcagggcctgagtggagagccccagccccggggccgcgccttcctggctgaagtgaagcagatgcgtgcacggaggaagacagccaagatgctgatggtggtgctgctggtcttcgccctctgctacctgcccatcagcgtcctcaatgtccttaagagggtgttcgggatgttccgccaagccagtgaccgcgaagctgtctacgcctgcttcaccttctcccactggctggtgtacgccaacagcgctgccaaccccatcatctacaacttcctcagtggcaaattccgggagcagtttaaggctgccttctcctgctgcctgcctggcctgggtccctgcggctctctgaaggcccctagtccccgctcctctgccagccacaagtccttgtccttgcagagccgatgctccatctccaaaatctctgagcatgtggtgctcaccagcgtcaccacagtgctgccctgagcgagggctgccctggaggctccggctcgggggatctgcccctacccctcatggaaagacagctggatgtggtgaaaggctgtggcttcagtcctgggtttctgcctgtgtgactctggataagtcacttcct

SEQ ID NO: 9 - protein, 425 aa, Homo sapiens
MEPSATPGAQMGVPPGSREPSPVPPDYEDEFLRYLWRDYLYPKQYEWVLIAAYVAVFVVALVGNTLVCLAVWRNHHMRTVTNYFIVNLSLADVLVTAICLPASLLVDITESWLFGHALCKVIPYLQAVSVSVAVLTLSFIALDRWYAICHPLLFKSTARRARGSILGIWAVSLAIMVPQAAVMECSSVLPELANRTRLFSVCDERWADDLYPKIYHSCFFIVTYLAPLGLMAMAYFQIFRKLWGRQIPGTTSALVRNWKRPSDQLGDLEQGLSGEPQPRGRAFLAEVKQMRARRKTAKMLMVVLLVFALCYLPISVLNVLKRVFGMFRQASDREAVYACFTFSHWLVYANSAANPIIYNFLSGKFREQFKAAFSCCLPGLGPCGSLKAPSPRSSASHKSLSLQSRCSISKISEHVVLTSVTTVLP

SEQ ID NO: 10 - DNA, 1843 bp, Homo sapiens
ggggggggggtaattgagcttcagctgagccggacgtagctttctcctcctggtgtcattgctgcagcctccagtgccgggtccctagttcctcagctgcctatcttcccggtgcaacatcgcctgtaaagacagcaaagccaccgcagaagttgcccggcagaagactccggaggcattggctcagtaacttttcacgtcattttctgctcgggagccccttctagcctctccgcgcagcctttcccaccgcaaatcaccagtgctcatggggcaggcggagaggagcttgcagcattgagcggaaccggacttgagcccgtgatgtccggcaccaaattggaggactcccccccttgtcgcaactggtcatctgcttcggagctgaatgaaactcaagagccctttttaaaccccaccgactatgacgacgaggaattcctgcggtacctgtggagggaatacctgcacccgaaagaatatgagtgggtcctgatcgccgggtacatcatcgtgttcgtcgtggctctcattgggaacgtcctggtttgtgtggcagtgtggaagaaccaccacatgaggacggtaaccaactacttcatagtcaatctttctctggctgatgtgctcgtgaccatcacctgccttccagccacactggtcgtggatatcactgagacctggttttttggacagtccctttgcaaagtgattccttatctacagaccgtgtcggtgtctgtgtctgtcctcacactgagctgtatcgccttggatcggtggtatgcaatctgtcaccctttgatgtttaagagcacagcaaagcgggcccgtaacagcattgtcatcatctggattgtctcctgcattataatgattcctcaggccatcgtcatggagtgcagcaccgtgttcccaggcttagccaataaaaccaccctctttacggtgtgtgatgagcgctggggtggtgaaatttatcccaagatgtaccacatctgtttctttctggtgacatacatggcaccactgtgtctcatggtgttggcttatctgcaaatatttcgcaaactctggtgtcgacagatccctggaacatcatctgtagttcagagaaaatggaagcccctgcagcctgtttcacagcctcgagggccaggacagccaacgaagtcccggatgagcgctgtggcggctgaaataaagcagatccgagccagaaggaaaacagcccggatgttgatggttgtgcttttggtatttgcaatttgctatctaccaattagcatcctcaatgtgctaaagagagtatttgggatgtttgcccatactgaagacagagagactgtgtatgcctggtttaccttttcacactggcttgtatatgccaatagtgctgcgaatccaattatttataattttctcagtggaaaatttcgagaggaatttaaagctgcgttttcttgctgttgccttggagttcaccatcgccaggaggatcggctcaccaggggacgaactagcacagagagccggaagtccttgaccactcaaatcagcaactttgataacatatcaaaactttctgagcaagttgtgctcactagcataagcacactcccagcagccaatggagcaggaccacttcaaaactggtagaatatttattcatatgacaaggatacctgagtaaaactatcctttttaaaatcactgggaacagaaattttattatcctatgatgtgaagctaaaattacttgtggatcttttttttttttaatctattgctctttggaaataaaaaaaaagtcagtttaaaatgaaaaaaaaaaaaaaaaaaa

SEQ ID NO: 11 - protein, 444 aa, Homo sapiens
MSGTKLEDSPPCRNWSSASELNETQEPFLNPTDYDDEEFLRYLWREYLHPKEYEWVLIAGYIIVFVVALIGNVLVCVAVWKNHHMRTVTNYFIVNLSLADVLVTITCLPATLVVDITETWFFGQSLCKVIPYLQTVSVSVSVLTLSCIALDRWYAICHPLMFKSTAKRARNSIVIIWIVSCIIMIPQAIVMECSTVFPGLANKTTLFTVCDERWGGEIYPKMYHICFFLVTYMAPLCLMVLAYLQIFRKLWCRQIPGTSSVVQRKWKPLQPVSQPRGPGQPTKSRMSAVAAEIKQIRARRKTARMLMVVLLVFAICYLPISILNVLKRVFGMFAHTEDRETVYAWFTFSHWLVYANSAANPIIYNFLSGKFREEFKAAFSCCCLGVHHRQEDRLTRGRTSTESRKSLTTQISNFDNISKLSEQVVLTSISTLPAANGAGPLQNW

SEQ ID NO: 12 - DNA, 1570 bp, Homo sapiens
ggcacgagggagagctggaggggcgtgcgcgcgccctcgctctgttgcgcgcgcggtgtcaccttgggcgcgagcggggccgcgcgcgcacgggacccggagccgagggccattgagtggcgatggcggcgacggcgagtgccggggccggcgggatagacgggaagccccgtacctcccctaagtccgtcaagttcctgtttgggggcctggccgggatgggagctacagtttttgtccagcccctggacctggtgaagaaccggatgcagttgagcggggaaggggccaagactcgagagtacaaaaccagcttccatgccctcaccagtatcctgaaggcagaaggcctgaggggcatttacactgggctgtcggctggcctgctgcgtcaggccacctacaccactacccgccttggcatctataccgtgctgtttgagcgcctgactggggctgatggtactccccctggctttctgctgaaggctgtgattggcatgaccgcaggtgccactggtgcctttgtgggaacaccagccgaagtggctcttatccgcatgactgccgatggccggcttccagctgaccagcgccgtggctacaaaaatgtgtttaacgccctgattcgaatcacccgggaagagggtgtcctcacactgtggcggggctgcatccctaccatggctcgggccgtcgtcgtcaatgctgcccagctcgcctcctactcccaatccaagcagttcttactggactcaggctacttctctgacaacatcttgtgccacttctgtgccagcatgatcagcggtcttgtcaccactgctgcctccatgcctgtggacattgccaagacccgaatccagaacatgcggatgattgatgggaagccggaatacaagaacgggctggacgtgctgttcaaagttgtccgctacgagggcttcttcagcctgtggaagggcttcacgccgtactatgcccgcctgggcccccacaccgtcctcaccttcatcttcttggagcagatgaacaaggcctacaagcgtctcttcctcagtggctgaagcggccgggggctcccactcgcctgctgcgcctatagccactgcgccctgggggcctgggctctgctgccctggacccctctatttatttcccttccacagtgtggtttcttcctctgcggtaaaggacttggtctgttctaccccctgctccagcttgccctgctcgtcctgatcctgtgatttctctgtccttggctattcttgcagggagctggaaaacttcctgaggatttctggcctccccctgggttttagtttcagggcacacaggacagcagaagatcccctttgtcagtggggaaaccaaggcagagctgaggggacagggaggagcagaagccatcaagatggtcaaagggcctgcagagggagatgtggcccttcctccccctcattgaggacttaataaattggattgatgacaccagcaaaaaaaaaaaaaaaaaaaaaaaaaaaaaaaaaaaaaaaaaaaaaaaaaaaaaaaaaaaaaaaaaaaaaaa

SEQ ID NO: 13 - protein, 314 aa, Homo sapiens
MAATASAGAGGIDGKPRTSPKSVKFLFGGLAGMGATVFVQPLDLVKNRMQLSGEGAKTREYKTSFHALTSILKAEGLRGIYTGLSAGLLRQATYTTTRLGIYTVLFERLTGADGTPPGFLLKAVIGMTAGATGAFVGTPAEVALIRMTADGRLPADQRRGYKNVFNALIRITREEGVLTLWRGCIPTMARAVVVNAAQLASYSQSKQFLLDSGYFSDNILCHFCASMISGLVTTAASMPVDIAKTRIQNMRMIDGKPEYKNGLDVLFKVVRYEGFFSLWKGFTPYYARLGPHTVLTFIFLEQMNKAYKRLFLSG

SEQ ID NO: 14 - DNA, 1743 bp, Homo sapiens
ccaggtcggccgtggtagcgtagggttgcgcggcccggaaacgcagagccggccaaagagcggcgcgacgtgagccggggccgtgcgcgaagagacctcgcgggcgcggagcgaaaggccggcgtgagtgagcgcggagacagtggccgccggcggcccaacccgtctatcccttcggccgccgccggcatgagccacatccagatcccgccggggctcacggagctgctgcagggctacacggtggaggtgctgcgacagcagccgcctgacctcgtcgaattcgcagtggagtacttcacccgcctgcgcgaggcccgcgccccagcctcagtcctgcccgccgccaccccacgccagagcctgggccaccccccgccagaacccggcccggaccgtgtcgccgacgccaaaggggacagcgagtcggaggaggacgaggacttggaagttccagttcctagcagatttaatagacgagtatcagtctgtgctgagacctataaccctgatgaggaagaggaagatacagatccaagggtgattcatcctaaaactgatgaacagagatgcagacttcaggaagcttgcaaagatattctccttttcaaaaatcttgatcaggaacagctttctcaagttctcgatgccatgtttgaaaggatagtcaaagctgatgagcatgtcattgaccaaggagatgatggagacaacttttatgtcatagaacggggaacttatgacattttagtaacaaaagataatcaaacccgctctgttggtcaatatgacaaccgtggcagttttggagaactagctctgatgtacaacaccccgagagctgctaccattgttgctacctcagaaggctccctttggggactggaccgggtgacttttagaagaatcatagtgaaaaataatgcaaagaagaggaagatgtttgaatcatttattgagtctgtgcccctccttaaatcactagaggtgtcagaacgaatgaagattgtggatgtaataggagagaagatctataaggatggagaacgcataatcactcagggtgaaaaggctgatagcttttacatcatagagtctggcgaagtgagcatcttgattagaagcaggactaaatcaaacaaggatggtgggaaccaggaggtcgagattgcccgctgccataaggggcagtactttggagagcttgccctggtcaccaacaaacccagagctgcctcagcttatgcagttggagatgtcaaatgcttagttatggatgtacaagcattcgagaggcttctggggccctgcatggacatcatgaagaggaacatctcacactatgaggaacagctggtgaagatgtttggctccagcgtggatctgggcaacctcgggcagtaggtgtgccacaccccagagccttcttagtgtgacaccaaaaccttctggtcagccacagaacacatacagaaaacagacatgacagaactgttcctgccgttgccgccactgctgccattgctgtggttatgggcatttagaaaacttgaaagtcagcactaaaggatgggcagaggttcaacccacacctccactttgcttctgaaggcccattcattagaccacttgtaaagattactccaacccagtttttatatctttggttcaaaacggcatgtctctccaacaatttaagtgcctgatacaaagtccaaagtataaacatgctcctttcctctc

SEQ ID NO: 15 - protein, 404 aa, Homo sapiens
MSHIQIPPGLTELLQGYTVEVLRQQPPDLVEFAVEYFTRLREARAPASVLPAATPRQSLGHPPPEPGPDRVADAKGDSESEEDEDLEVPVPSRFNRRVSVCAETYNPDEEEEDTDPRVIHPKTDEQRCRLQEACKDILLFKNLDQEQLSQVLDAMFERIVKADEHVIDQGDDGDNFYVIERGTYDILVTKDNQTRSVGQYDNRGSFGELALMYNTPRAATIVATSEGSLWGLDRVTFRRIIVKNNAKKRKMFESFIESVPLLKSLEVSERMKIVDVIGEKIYKDGERIITQGEKADSFYIIESGEVSILIRSRTKSNKDGGNQEVEIARCHKGQYFGELALVTNKPRAASAYAVGDVKCLVMDVQAFERLLGPCMDIMKRNISHYEEQLVKMFGSSVDLGNLGQ

SEQ ID NO: 16 - DNA, 3259 bp, Homo sapiens
gacgcgcgccgggagccggcggccgggccagccggcgccggggcccagtgcgccgcgctcgcagccggtagcgcgccagccgtaggcgtcgctcggcagccgcggggccctaggcgtgccggggagggggcgagggcggccaggcgcctgccgccccggaggcaggatgagcatcgagatcccggcgggactgacggagctgctgcagggcttcacggtggaggtgctgaggcaccagcccgcggacctgctggagttcgcgctgcagcacttcacccgcctgcagcaggagaacgagcgcaaaggcaccgcgcgcttcggccatgagggcaggacctggggggacctgggcgccgctgccgggggcggcacccccagcaagggggtcaacttcgccgaggagcccatgcagtccgactccgaggacggggaggaggaggaggcggcgcccgcggacgcaggggcgttcaatgctccagtaataaaccgattcacaaggcgtgcctcagtatgtgcagaagcttataatcctgatgaagaagaagatgatgcagagtccaggattatacatccaaaaactgatgatcaaagaaataggttgcaagaggcttgcaaagacatcctgctgtttaagaatctggatccggagcagatgtctcaagtattagatgccatgtttgaaaaattggtcaaagatggggagcatgtaattgatcaaggtgacgatggtgacaacttttatgtaattgatagaggcacatttgatatttatgtgaaatgtgatggtgttggaagatgtgttggtaactatgataatcgtgggagtttcggcgaactggccttaatgtacaatacacccagagcagctacaatcactgctacctctcctggtgctctgtggggtttggacagggtaaccttcaggagaataattgtgaaaaacaatgccaaaaagagaaaaatgtatgaaagctttattgagtcactgccattccttaaatctttggagttttctgaacgcctgaaagtagtagatgtgataggcaccaaagtatacaacgatggagaacaaatcattgctcagggagattcggctgattcttttttcattgtagaatctggagaagtgaaaattactatgaaaagaaagggtaaatcagaagtggaagagaatggtgcagtagaaatgcctcgatgctcgcggggacagtactttggagagcttgccctggtaactaacaaacctcgagcagcttctgcccacgccattgggactgtcaaatgtttagcaatggatgtgcaagcatttgaaaggcttctgggaccttgcatggaaattatgaaaaggaacatcgctacctatgaagaacagttagttgccctgtttggaacgaacatggatattgttgaacccactgcatgaagcaaaagtatggagcaagacctgtagtgacaaaattacacagtagtggttagtccactgagaatgtgtttgtgtagatgccaagcattttctgtgatttcaggttttttcctttttttacatttacaacgtatcaataaacagtagtgatttaatagtcaataggctttaacatcactttctaaagagtagttcataaaaaaatcaacatactgataaaatgactttgtactccacaaaattatgactgaaaggtttattaaaatgattgtaatatatagaaagtatctgtgtttaagaagataattaaaggatgttatcataggctatatgtgttttacttattcagactgataatcatattagtgactatccccatgtaagagggcacttggcaattaaacatgctacacagcatggcatcacttttttttataactcattaaacacagtaaaattttaatcatttttgttttaaagttttctagcttgataagttatgtgctgccttggcctattggtgaaatggtataaaatatcatatgcagttttaaaactttttatatttttgcaataaagtacattttgactttgttggcataatgtcagtaacatacatattccagtggttttatggacaggcaatttagtcattatgataataaggaaaacagtgttttagatgagagatcattaatgcatttttccctcatcaagcatatatctgcttttttttattttgcaattctctgtattctatgtctttaaaaatttgatcttgacatttaatgtcacaaagttttgtttttttaaaaagtgatttaaacttaagatccgacattttttgtattctttaagattttacacctaaaaaatctctcctatcccaaaaataatgtgggatccttatcagcatgcccacagtttatttctttgttcttcactaggcctgcataatacagtcctatgtagacatctgttcccttgggtttccgttctttcttaggatggttgccaacccacaatctcattgatcagcagccaatatgggtttgtttggtttttttaattcttaaaaacatcctctagaggaatagaaacaaatttttatgagcataaccctatataaagacaaaatgaatttctgaccttaccatatataccattaggccttgccattgctttaatgtagactcatagttgaaattagtgcagaaagaactcagatgtactagattttcattgttcattgatatgctcagtatgctgccacataagatgaatttaattatattcaaccaaagcaatatactcttacatgatttctaggccccatgacccagtgtctagagacattaattctaaccagttgtttgcttttaaatgagtgatttcattttgggaaacaggtttcaaatgaatatatatacatgggtaaaattactctgtgctagtgtagtcttactagagaatgtttatggtcccacttgtatatgaaaatgtggttagaatgttaattggataatgtatatataagaagttaaagtatgtaaagtataacttcagccacatttttagaacactgtttaacatttttgcaaaaccttcttgtaggaaaagagagctctctacatgaagatgacttgttttatatttcagattttattttaaaagccatgtctgttaaacaagaaaaaacacaaaagaactccagattcctggttcatcattctgtattcttactcactttttcaagttatctattttgttgcataaactaattgttaactattcatggaacagcaaacgcctgtttaataaagaactttgaccaag

SEQ ID NO: 17 - protein, 418 aa, Homo sapiens
MSIEIPAGLTELLQGFTVEVLRHQPADLLEFALQHFTRLQQENERKGTARFGHEGRTWGDLGAAAGGGTPSKGVNFAEEPMQSDSEDGEEEEAAPADAGAFNAPVINRFTRRASVCAEAYNPDEEEDDAESRIIHPKTDDQRNRLQEACKDILLFKNLDPEQMSQVLDAMFEKLVKDGEHVIDQGDDGDNFYVIDRGTFDIYVKCDGVGRCVGNYDNRGSFGELALMYNTPRAATITATSPGALWGLDRVTFRRIIVKNNAKKRKMYESFIESLPFLKSLEFSERLKVVDVIGTKVYNDGEQIIAQGDSADSFFIVESGEVKITMKRKGKSEVEENGAVEMPRCSRGQYFGELALVTNKPRAASAHAIGTVKCLAMDVQAFERLLGPCMEIMKRNIATYEEQLVALFGTNMDIVEPTA

SEQ ID NO: 18 - DNA, 1985 bp, Homo sapiens
gcaccgcccccgcccgcaagaaagatggcagtggcctgatccgggcccgttggcggcgtcactgacgcttcgctccggtcctcggatcccgagcgcggggaggcagaccgactgtgagctgcttgtccccatcctgcggccgtcctggggacacagagccctccgtggtgcccggggattggattggagccaggacctcacttcctcctctgcccctgcccctgcccctcccagcacctggcccacaccctgcagcccgccccatggtctggccctgggtggcgatggcgtccaggtggggtcccctcattggcctggctccgtgctgcctctggctcctgggggcagtccttctgatggacgcgtctgcacggcctgccaaccactcgtccactcgagagagagtagccaacagggaggagaatgagatcctgcccccagaccacctgaacggggtgaagctggagatggacgggcacctcaatcgcggcttccaccaggaggtcttcctaggcaaggacctgggtggctttgatgaggacgcggagccgcggcggagccggaggaagctgatggtcatcttttccaaggtggatgtgaacactgaccggaagatcagtgccaaggagatgcagcgctggatcatggagaagacggccgagcacttccaggaggccatggaggagagcaagacacacttccgcgccgtggaccctgacggggacggtcacgtgtcttgggacgagtataaggtgaagtttttggcgagtaaaggccatagcgagaaggaggttgccgacgccatcaggctcaacgaggaactcaaagtggacgaggaaacacaggaagtcctggagaacctgaaggaccgctggtaccaggcggacagcccccctgcagacctgctgctgacggaggaggagttcctgtcgttcctccaccccgagcacagccggggaatgctcaggttcatggtgaaggagatcgtccgggacctggaccaggacggtgacaagcagctctctgtgcccgagttcatctccctgcccgtgggcaccgtggagaaccagcagggccaggacattgacgacaactgggtgaaagacagaaaaaaggagtttgaggagctcattgactccaaccacgacggcatcgtgaccgccgaggagctggagagctacatggaccccatgaacgagtacaacgcgctgaacgaggccaagcagatgatcgccgtcgccgacgagaaccagaaccaccacctggagcccgaggaggtgctcaagtacagcgagttcttcacgggcagcaagctggtggactacgcgcgcagcgtgcacgaggagttttgagcgcccggccgcgccccgcgccgccccccacgcaccaccggggcggcctcgcgggtgactccgggctccgtggctgtcccggaccccacctcttccctgccgcccgccaccggccgaccgaccgcggctgccccagttgatgagcggcgtgtcccctctgcagcgcgcaccccggcggggctttggctgtgacgcggtcggggcgcggggctgggctgtggccccgcggcgccgcctcctccctggtccctcgaaatcgtggcatctcacttctgagaacgaaatctcgcttcagtcactctgccgaaggcgctgacggcatcgcggccggaacctctgggcccggcccctcccagggccgccgctccgtgggaaaaaacagctcctccatttccttgaaaactgaacgattattaaaaatagattaaacttcgctggaaatgagtagccaggaagttcaggggagggtgccgggtccttcccgggcctggcgtgtcggagccacccaggtcccgcagctgccgctgagaaaatgcaaatatttgttgtgacaagaatcacatacatttactttaaatatagttgccttttttggtcagcttcaaaaaaaaaaaaaaaaaaaaaaaaaaaaaa

SEQ ID NO: 19 - protein, 362 aa, Homo sapiens
MVWPWVAMASRWGPLIGLAPCCLWLLGAVLLMDASARPANHSSTRERVANREENEILPPDHLNGVKLEMDGHLNRGFHQEVFLGKDLGGFDEDAEPRRSRRKLMVIFSKVDVNTDRKISAKEMQRWIMEKTAEHFQEAMEESKTHFRAVDPDGDGHVSWDEYKVKFLASKGHSEKEVADAIRLNEELKVDEETQEVLENLKDRWYQADSPPADLLLTEEEFLSFLHPEHSRGMLRFMVKEIVRDLDQDGDKQLSVPEFISLPVGTVENQQGQDIDDNWVKDRKKEFEELIDSNHDGIVTAEELESYMDPMNEYNALNEAKQMIAVADENQNHHLEPEEVLKYSEFFTGSKLVDYARSVHEEF

SEQ ID NO: 20 - DNA, 2092 bp, Homo sapiens
ggcgtcccctccgtgaggtcgcgcccgttcgcaccgcccccgcccgcaagaaagatggcagtggcctgatccgggcccgttggcggcgtcactgacgcttcgctccggtcctcggatcccgagcgcggggaggcagaccgactgtgagctgcttgtccccatcctgcggccgtcctggggacacagagccctccgtggtgcccggggattggattggagccaggacctcacttcctcctctgcccctgcccctgcccctcccagcacctggcccacaccctgcagcccgccccatggtctggccctgggtggcgatggcgtccaggtggggtcccctcattggcctggctccgtgctgcctctggctcctgggggcagtccttctgatggacgcgtctgcacggcctgccaaccactcgtccactcgagagagagtagccaacagggaggagaatgagatcctgcccccagaccacctgaacggggtgaagctggagatggacgggcacctcaatcgcggcttccaccaggaggtcttcctaggcaaggacctgggtggctttgatgaggacgcggagccgcggcggagccggaggaagctgatggtcatcttttccaaggtggatgtgaacactgaccggaagatcagtgccaaggagatgcagcgctggatcatggagaagacggccgagcacttccaggaggccatggaggagagcaagacacacttccgcgccgtggaccctgacggggacggtcacgtgtcttgggacgagtataaggtgaagtttttggcgagtaaaggccatagcgagaaggaggttgccgacgccatcaggctcaacgaggaactcaaagtggacgaggaaacacaggaagtcctggagaacctgaaggaccgctggtaccaggcggacagcccccctgcagacctgctgctgacggaggaggagttcctgtcgttcctccaccccgagcacagccggggaatgctcaggttcatggtgaaggagatcgtccgggacctggaccaggacggtgacaagcagctctctgtgcccgagttcatctccctgcccgtgggcaccgtggagaaccagcagggccaggacattgacgacaactgggtgaaagacagaaaaaaggagtttgaggagctcattgactccaaccacgacggcatcgtgaccgccgaggagctggagaacgtgcccacactcccgctgcagccaataggcaccttaaatagccacttcgtgcggctggccgcggagctcggagggggaaaggcgacgctgacctgtgccccgctcgcccgcagagctacatggaccccatgaacgagtacaacgcgctgaacgaggccaagcagatgatcgccgtcgccgacgagaaccagaaccaccacctggagcccgaggaggtgctcaagtacagcgagttcttcacgggcagcaagctggtggactacgcgcgcagcgtgcacgaggagttttgagcgcccggccgcgccccgcgccgccccccacgcaccaccgggggggcctcgcgggtgactccggcctccgtggctgtcccggaccccacctcttctctgccgcccaccaccggccgaccgaccgcggcttccccagttgatgagcggcgtgtcccctctgcagcgcgcaccccggcggggctttggctgtgacgcggtcggggcgcggggctggtctgtggccccgcggcgcgcctcctccctggtccctcgaaatcgtggcatctcacttctgagaacgaaatctcgcttcagtcactctgccgaaggcgctgacggcatcgcggccggaacctctgggcccggcccctcccagggccgccgctccgtgggaaaaaacagctcctccatttccttggaaactgaacgattattaaaaatagataaacttcgctggaaatgagtagccaggaagttcaggggagggtccggggtccttcccggggcctggcgtgtcggaaccacccaggtcccgcagctgcctctgagaaaatccaaatattttttgtgacaagaatcacaaacatttactttaaatataaaaaaaaaaaaaaa

SEQ ID NO: 21 - protein, 348 aa, Homo sapiens
MVWPWVAMASRWGPLIGLAPCCLWLLGAVLLMDASARPANHSSTRERVANREENEILPPDHLNGVKLEMDGHLNRGFHQEVFLGKDLGGFDEDAEPRRSRRKLMVIFSKVDVNTDRKISAKEMQRWIMEKTAEHFQEAMEESKTHFRAVDPDGDGHVSWDEYKVKFLASKGHSEKEVADAIRLNEELKVDEETQEVLENLKDRWYQADSPPADLLLTEEEFLSFLHPEHSRGMLRFMVKEIVRDLDQDGDKQLSVPEFISLPVGTVENQQGQDIDDNWVKDRKKEFEELIDSNHDGIVTAEELENVPTLPLQPIGTLNSHFVRLAAELGGGKATLTCAPLARRATWTP

SEQ ID NO: 22 - DNA, 1605 bp, Homo sapiens
gcggcgcgctgcaggcgcggggaacaccaatggcggggtacttgaagctggtgtgtgtttcctttcagcgtcaagggttccacactgttgggagtcgctgcaagaatcggacaggcgctgagcacctgtggctgacccgacatctcagggacccatttgtgaaggctgcgaaggtggagagttaccggtgtcgaagcgccttcaagctcctggaggtgaacgagaggcaccagattctgcggcccggccttcgggtgttagactgtggggcagctcctggggcctggagtcaggtggcggtgcagaaggtcaacgccgcaggcacagatcccagctctcctgttggcttcgtgcttggggtagatcttcttcacatattccccctggaaggagcaacttttctgtgccctgctgacgtgactgacccgagaacctcacagagaatcctcgaggtgcttcctggcaggagagcagatgtgattctgagcgacatggcgcccaatgccacagggttccgggacctcgatcatgacaggctcatcagcctgtgcctgacccttctcagcgtgaccccagacatcctgcaacctggggggacattcctttgtaaaacctgggctggaagtcaaagccgtcggttacagaggagactgacagaggaattccagaatgtaaggatcatcaaacctgaagccagcaggaaagagtcatcagaagtgtacttcttggccacacagtaccacggaaggaagggcactgtgaagcagtgaggatttcttgtgccattttcataatggtcattagctccttttaagctagaaacgtagcctgagctcctgaagagttcctgggagatttgagctgattttggagatggagcaggacaagtggggagtctctctctctctttctctctctctctttttaaccaaaaagagatgacaaaactaagttcaggggccatggaaaatgaaaaagtccgctatattgtgatttgggaagagaaagttatcaagagaaagaggtgaggatggaaggatggagaaaaacagactgtgggaaggatcagaaggaatccgccgaggcagggatgggtgtgcccatgtgtgccttgacgggacttcatcttatagactgttaaactgtcacacacaaacaggctttccacccctgctctgagagcaccacgcacagatttccagttcttagtgtggctgtttaaagtagaaaatctgggggctgggtgaggccactcatgcctgtaaacccagggctttagaaggctgaggctgggggattgcttgaagtcaggagttcaagaccaacctgggcaacatagcaacaccccccatgtctacaaaaatgaaaaaccaaaaagcaaaccaaaagaaaaatctgaaatttccatctggggattaacttctgtctttctggtgaacaatatagcaattcacgcattcttcaagcagcaaaagttcccggaacaattagggaagacgtatggtctgaatttatccaggcagtgggtctgctttggtttttgctggaaatttatatcagtgtctgggctcccaagaacataaatgtaattgccaaagcaaaaaa

SEQ ID NO: 23 - protein, 246 aa, Homo sapiens
MAGYLKLVCVSFQRQGFHTVGSRCKNRTGAEHLWLTRHLRDPFVKAAKVESYRCRSAFKLLEVNERHQILRPGLRVLDCGAAPGAWSQVAVQKVNAAGTDPSSPVGFVLGVDLLHIFPLEGATFLCPADVTDPRTSQRILEVLPGRRADVILSDMAPNATGFRDLDHDRLISLCLTLLSVTPDILQPGGTFLCKTWAGSQSRRLQRRLTEEFQNVRIIKPEASRKESSEVYFLATQYHGRKGTVKQ

SEQ ID NO: 24 - DNA, 1709 bp, Homo sapiens
aaacagcggacggaggggccggcggtggcggagccggagcaagcaggggttcggcggcattacctgtacccattcaccggcggctaccggcggcggcgcgcagcgtgtcaggcggagagacccgccgccaggaatgaatctgaagtctgctgcagtaaaacacagaaggctttaaaatgttttcttgcataaaattcaaaacttttaagtagctgcttatgagaatagggaaggcagaaagctaatgtctgtctcaagatacaggacagctgtttgctcatcaacctcaactgtgtgtgcaactgaggaacatggctcaagaaactaatcacagccaagtgcctatgctttgttccactggctgtggattttatggaaaccctcgtacaaatggcatgtgttcagtatgctataaagaacatcttcaaagacagaatagtagtaatggtagaataagcccacctgcaacctctgtcagtagtctgtctgaatctttaccagttcaatgcacagatggcagtgtgccagaagcccagtcagcattagactctacatcttcatctatgcagcccagccctgtatcaaatcagtcacttttatcagaatctgtagcatcttctcaattggacagtacatctgtggacaaagcagtacctgaaacagaagatgtgcaggcttcagtatcagacacagcacagcagccatctgaagagcaaagcaagtctcttgaaaaaccgaaacaaaaaaagaatcgctgtttcatgtgcaggaagaaagtgggacttactgggtttgaatgccggtgtggaaatgtttactgtggtgtacaccgttactcagatgtacacaattgctcttacaattacaaagccgatgctgctgagaaaatcagaaaagaaaatccagtagttgttggtgaaaagatccaaaagatttgaactcctgctggaatacaaaattcttgagcatctgcaaactaaaaattgacttgaggttttttttttcctagtcattgggaatgtagagcagtgtatcttgcatgtcatcggaagaatagatttttgttttggttttgttttgaaaatgactctgaacatttatttccattgcaatttctgtggctgaggagacttaaactttacaagtattatccttttaagatcattttaattttagttgagtgcagagggcttttataacaaacgtgcagaaattttggagggctgtgatttttccagtattaaacatgcatgcattaatcttgcagtttattttctcattgtgtatgtatatatcgcttttctctgcagcacgatttctcttttgataatgccctttagggcacaactagttatcagtaactgaatgtatcttaatcattatggctgcttctgttttttcattaacaaaggttattcatatgttagcatatagtttctttgcacccactatttatgtctgaatcatttgtcacaagagagtgtgtgctgatgagattgtaagtttgtgtgtttaaacttttttttgagcgagggaagaaaaagctgtatgcatttcattgctgtctacaggtttctttcagattatgttcatgggtttgtgtgtatacaatatgaagaatgatctgaagtaattgtgctgtatttatgtttattcaccagtctttgattaaattaaatgaaaaaaaaaaaaaaaaaaaaaaaaaaaaa

SEQ ID NO: 25 - protein, 208 aa, Homo sapiens
MAQETNHSQVPMLCSTGCGFYGNPRTNGMCSVCYKEHLQRQNSSNGRISPPATSVSSLSESLPVQCTDGSVPEAQSALDSTSSSMQPSPVSNQSLLSESVASSQLDSTSVDKAVPETEDVQASVSDTAQQPSEEQSKSLEKPKQKKNRCFMCRKKVGLTGFECRCGNVYCGVHRYSDVHNCSYNYKADAAEKIRKENPVVVGEKIQKI

SEQ ID NO: 26 - DNA, 2425 bp, Homo sapiens
gcggccgccggtcctccctccacctcctcctcggccccccctcgcttccctcctcccacttcccgagctccggcgtcgtcccggccacgctcgacgctgctgcaggaacaaaggaagaccccgcggcggcggcggcgccacctccgcctgctgctccgacccgctcccggcccgcggcggcggcaccagggcgcccggctcagccttcccggaggcctcggcccggcctcatcgtgccggcttcgcgcgcgaacccggctttcgcatttgggaccctgcaggaaaaatatggctcaggagactaaccagaccccggggcccatgctgtgtagcacaggatgtggcttttatggaaatcctaggacaaatggaatgtgttcagtttgctacaaagaacatcttcagaggcagcaaaatagtggcagaatgagcccaatggggacagctagtggttccaacagtcctacctcagattctgcatctgtacagagagcagacactagcttaaacaactgtgaaggtgctgctggcagcacatctgaaaaatcaagaaatgtgcctgtggctgccttgcctgtaactcagcaaatgacagaaatgagcatttcaagagaggacaaaataactaccccgaaaacagaggtgtcagagccagttgtcactcagcccagtccatcagtttctcagcccagtacttctcagagtgaagaaaaagctcctgaattgcccaaaccaaagaaaaacagatgtttcatgtgcagaaagaaagttggtcttacagggtttgactgccgatgtggaaatttgttttgtggacttcaccgttactctgacaagcacaactgtccgtatgattacaaagcagaagctgcagcaaaaatcagaaaagagaatccagttgttgtggctgaaaaaattcagagaatataaattacttcttgtgaagagactgaaactttgtttttattttaatatatcgtaggaaaacattaaagagcagatgcatggccatttttctttgatgttctccagagttttacattacacttgtctgtcttataattgatattttaggatgtttgggtgtttgttacaggcagaattggatagatacagccctacaaatgtatatgccctcccctgaaaaaaattggatgaaaatctgcacagcaaagtgaaacacacagataataggaacaaaatgtagttcccatgtgccaaacaaaataaatgaaatctctgcatgtttgcagcatatctgccttttgggaatgtaatcaaggtataatctttggctagtgttatgtgcctgtatttttttaaaatggtacaccagaaaaggactggcagtctacttctaccatagttaaacttcaccctctttaatttcacaacatattctttggaagcaggaagaaatgctcataaagaggatcagaccttctttcccgtgaaaccagtatttggcgccatatataagcctggttaaattggtcatctaaagctgtcaaataagacattctgtgaaaggtaaacatcgaaactggttataagtaaaaccatcaagccaacaacagggtcttgagataacctttgaagcttattgtactggcctgcaccagaagatgtctgcattactcattgctaaaaatgtgtagcacagaactgcactaggattaatttgtttacaagaagaaatttaaactctacgtttggttttcacatacagcagctctattgaataacatgcatctgaattttaagttgcaaaggtatctgaataatttttcatgtgcatcttttgtcgaatgttttggttcaagaaagaatgtttaaagctttttaaaagacttcagttcttaatgtaactgtacccttctgcatggaaaatcataaccaacatggctgcagtagacttcttagtggtatccagcgccacttgcagagggctgctttatcatattgtacttgggtgtaggactctagtgttcttgggtgtattgcatgggctgcattatctacagcattgtacaataacaactagaaaaggcagtatacttcactgatgcttgtctggtaataatcacttctgtgttataatggaaggttttttgtgatgtatgaaacttgtgttttttatatataaatgagtatagttagtgttgtggtaatgcctgttttcatctgtaaatagttaagtatgtacacgaggcactacttctgatttattgcaatgttcagtcctagtttttacttttattcttaaagcattcagttttgctttcaattttatgtaccttagttctgagttagacctgcagatgtgtacagatagttcatatttatgtattgcacataatcatgctattcagcattgatgctatattgtattatgtaaataataaaagccatgtacagagggaaa

SEQ ID NO: 27 - protein, 213 aa, Homo sapiens
MAQETNQTPGPMLCSTGCGFYGNPRTNGMCSVCYKEHLQRQQNSGRMSPMGTASGSNSPTSDSASVQRADTSLNNCEGAAGSTSEKSRNVPVAALPVTQQMTEMSISREDKITTPKTEVSEPVVTQPSPSVSQPSTSQSEEKAPELPKPKKNRCFMCRKKVGLTGFDCRCGNLFCGLHRYSDKHNCPYDYKAEAAAKIRKENPVVVAEKIQRI

SEQ ID NO: 28 - DNA, 17 bp, Artificial Sequence
  mouse spermidine synthase Taqman primer
ccgtgccgccttcgtac

SEQ ID NO: 29 - DNA, 24 bp, Artificial Sequence
  mouse spermidine synthase Taqman primer
acctggattcagcttatgtcattg

SEQ ID NO: 30 - DNA, 22 bp, Artificial Sequence
  mouse spermidine synthase Taqman probe
cctgagttcacccggaaggccc

SEQ ID NO: 31 - DNA, 19 bp, Artificial Sequence
  mouse RFamide-related peptide receptor Taqman primer
gcagctgcacttgctgtcc

SEQ ID NO: 32 - DNA, 19 bp, Artificial Sequence
  mouse RFamide-related peptide receptor Taqman primer
tgctgtggaagaaggccag

SEQ ID NO: 33 - DNA, 24 bp, Artificial Sequence
  mouse RFamide-related peptide receptor Taqman probe
tctacgccttcccgttggcacact

SEQ ID NO: 34 - DNA, 20 bp, Artificial Sequence
  mouse G protein-coupled receptor 74 Taqman primer
tgagagcgaaacgcaacatg

SEQ ID NO: 35 - DNA, 21 bp, Artificial Sequence
  mouse G protein-coupled receptor 74 Taqman primer
gttttgagacaccggttcctg

SEQ ID NO: 36 - DNA, 25 bp, Artificial Sequence
  mouse G protein-coupled receptor 74 Taqman probe
tcataaacacatcgggcctgctggt

SEQ ID NO: 37 - DNA, 20 bp, Artificial Sequence
  mouse solute carrier family 25 member 11 Taqman primer
catccctaccatggctcgag

SEQ ID NO: 38 - DNA, 26 bp, Artificial Sequence
  mouse solute carrier family 25 member 11 Taqman primer
tgcttagattgagagtaagaggcaag

SEQ ID NO: 39 - DNA, 21 bp, Artificial Sequence
  mouse solute carrier family 25 member 11 Taqman probe
tgtcgttgtcaatgccgccca

SEQ ID NO: 40 - DNA, 23 bp, Artificial Sequence
  mouse protein kinase, cAMP dependent regulatory, type II alpha Taqman primer
gccacaagaacacacacagaaaa

SEQ ID NO: 41 - DNA, 15 bp, Artificial Sequence
  mouse protein kinase, cAMP dependent regulatory, type II alpha Taqman primer
atggcagcggcggag

SEQ ID NO: 42 - DNA, 25 bp, Artificial Sequence
  mouse protein kinase, cAMP dependent regulatory, type II alpha Taqman probe
agacacgacagaaccgctcctgctg

SEQ ID NO: 43 - DNA, 20 bp, Artificial Sequence
  mouse Cab45 Taqman primer
cgcaacgtgcatgaagagtt

SEQ ID NO: 44 - DNA, 20 bp, Artificial Sequence
  mouse Cab45 Taqman primer
agacaggacactggcgacct

SEQ ID NO: 45 - DNA, 21 bp, Artificial Sequence
  mouse Cab45 Taqman probe
tgagcatgcccctcagctggg

SEQ ID NO: 46 - DNA, 20 bp, Artificial Sequence
  mouse cell division protein FtsJTaqman primer
gcaggactgaggaacaagcc

SEQ ID NO: 47 - DNA, 18 bp, Artificial Sequence
  mouse cell division protein FtsJTaqman primer
cctggaggcagcagcaac

SEQ ID NO: 48 - DNA, 25 bp, Artificial Sequence
  mouse cell division protein FtsJTaqman probe
catccataccccactccccgtctcc

SEQ ID NO: 49 - protein, 281 aa, Homo sapiens
SLNNGWFSELQADLWPGQSFSLKVKEVIHKEKSRFQDIQIVETETYGRCLILDGIIQCTARDEFSYQEMISFLPLCAHPNPKKVLIVGGGDGGVAREVVKHPLVEEVHQVEIDDRVVELSKQYLPAMACGFANEKLKLTIGDGFDYMKKHKNEFDVIITDSSDPIGPAVSLFQESYYELMKHALKDDGIVCSQGGSFWLDLDYIKKTMSGCKEHFAKVAYAVTSVPSYPCGHIGFVMGSLNKNQDFATPKLGKSEIDSIDLRYYSSEVHSAAFALPRWVQK

SEQ ID NO: 50 - protein, 282 aa, Homo sapiens
AIREGWFRETCSLWPGQALSLQVEQLLHHRRSRYQDILVFRSKTYGNVLVLDGVIQCTERDEFSYQEMIANLPLCSHPNPRKVLIIGGGDGGVLREVVKHPSVESVVQCEIDEDVIQVSKKFLPGMAIGYSSSKVTLHVGDGFEFMKQNQDAFDVIITDSSDPMGPAESLFKESYYQLMKTALKEDGVLCCQGECEWLHLDLIKEMRQFCQSLFPVVAYAYCTIPTYPSGQIGFMLCSKNPSTNFQEPVQPLTQQQVAQMQLKYYNSDVHRAAFVLPEFARK

SEQ ID NO: 51 - protein, 302 aa, Mus musculus
MEPGPDGPAAPGPAAIREGWFRETCSLWPGQALSLQVEQLLHHRRSRYQDILVFRSKTYGNVLVLDGVIQCTERDEFSYQEMIANLPLCSHPNPRKVLIIGGGDGGVLREVVKHPSVESVVQCEIDEDVIEVSKKFLPGMAVGFSSSKLTLHVGDGFEFMKQNQDAFDVIITDSSDPMGPAESLFKESYYQLMKTALKEDGILCCQGECQWLHLDLIKEMRHFCKSLFPVVDYAYCSIPTYPSGQIGFMLCSKNPSTNFREPVQQLTQAQVEQMQLKYYNSDMHRAAFVLPEFTRKALNDIS

SEQ ID NO: 52 - protein, 287 aa, Drosophila
MDSLNNGWFSELQADLWPGQSFSLKVKEVIHKEKSRFQDIQIVETETYGRCLILDGIIQCTARDEFSYQEMISFLPLCAHPNPKKVLIVGGGDGGVAREVVKHPLVEEVHQVEIDDRVVELSKQYLPAMACGFANEKLKLTIGDGFDYMKKHKNEFDVIITDSSDPIGPAVSLFQESYYELMKHALKDDGIVCSQGGSFWLDLDYIKKTMSGCKEHFAKVAYAVTSVPSYPCGHIGFVMGSLNKNQDFATPKLGKSEIDSIDLRYYSSEVHSAAFALPRWVQKHFYE

SEQ ID NO: 53 - protein, 419 aa, Homo sapiens
MNEKWDTNSSENWHPIWNVNDTKHHLYSDINITYVNYYLHQPQVAAIFIISYFLIFFLCMMGNTVVCFIVMRNKHMHTVTNLFILNLAISDLLVGIFCMPITLLDNIIAGWPFGNTMCKISGLVQGISVAASVFTLVAIAVDRFQCVVYPFKPKLTIKTAFVIIMIIWVLAITIMSPSAVMLHVQEEKYYRVRLNSQNKTSPVYWCREDWPNQEMRKIYTTVLFANIYLAPLSLIVIMYGRIGISFRAAVPHTGRKNQEQWHVVSRKKQKIIKMLLIVALLFILSWLPLWTLMMLSDYADLSPNELQIINIYIYPFAHWLAFGNSSVNPIIYGFFNENFRRGFQEAFQLQLCQKRAKPMEAYALKAKSHVLINTSNQLVQESTFQNPHGETLLYRKSAEKPQQELVMEELKETTNSSEI

SEQ ID NO: 54 - protein, 520 aa, Drosophila; identical to SEQ ID NO: 3

SEQ ID NO: 55 - protein, 294 aa, Drosophila
KTVPTHMKFVMGGTSGMLATCIVQPLDLLKTRMQISGTLGTREYKNSFEVLSKVLKNEGILSLYNGLSAGLLRQATYTSAKMGVYQMELDWYRKNFGNYPSMVASMTMGIVAGAFGAMCGNPAEVALIRMMSDNRLMPEDRRNYKNVGDAFVRIVKDEGVVALWRGCLPTVGRAMVVNMVQLASYSLMKNQLHGYLSEGIPLHLTAALVSGLLTSVTSMPLDMAKTRIQQMKVIDGKPEYSGTIDVLKKVLKNEGAFAVWKGFTPYLMRMGPHTIFSFVFLEQMNKAYSKHMLS

SEQ ID NO: 56 - protein, 297 aa, Homo sapiens
RTSPKSVKFLFGGLAGMGATVFVQPLDLVKNRMQLSGEGAKTREYKTSFHALTSILKAEGLRGIYTGLSAGLLRQATYTTTRLGIYTVLFERLTGADGTPPGFLLKAVIGMTAGATGAFVGTPAEVALIRMTADGRLPADQRRGYKNVFNALIRITREEGVLTLWRGCIPTMARAVVVNAAQLASYSQSKQFLLDSGYFSDNILCHFCASMISGLVTTAASMPVDIAKTRIQNMRMIDGKPEYKNGLDVLFKVVRYEGFFSLWKGFTPYYARLGPHTVLTFIFLEQMNKAYKRLFLS

SEQ ID NO: 57 - protein, 314 aa, Mus musculus
MAATASPGAGRMDGKPRTSPKSVKFLFGGLAGMGATVFVQPLDLVKNRMQLSGEGAKTREYKTSFHALTSILKTEGLKGIYTGLSAGLLRQATYTTTRLGIYTVLFERLTGADGTPPGFLLKALIGMTAGATGAFVGTPAEVALIRMTADGRLPADQRRGYKNVFNALVRIAREEGVPTLWRGCIPTMARAVVVNAAQLASYSQSKQFLLDSGYFSDNILCHFCASMISGLVTTAASMPVDIVKTRIQNMRMIDGKPEYKNGLDVLLKVVRYEGFFSLWKGFTPYYARLGPHTVLTFIFLEQMNKAYKRLFLSG

SEQ ID NO: 58 - protein, 311 aa, Drosophila
MALVYGVEKKTVPTHMKFVMGGTSGMLATCIVQPLDLLKTRMQISGTLGTREYKNSFEVLSKVLKNEGILSLYNGLSAGLLRQATYTSAKMGVYQMELDWYRKNFGNYPSMVASMTMGIVAGAFGAMCGNPAEVALIRMMSDNRLMPEDRRNYKNVGDAFVRIVKDEGVVALWRGCLPTVGRAMVVNMVQLASYSLMKNQLHGYLSEGIPLHLTAALVSGLLTSVTSMPLDMAKTRIQQMKVIDGKPEYSGTIDVLKKVLKNEGAFAVWKGFTPYLMRMGPHTIFSFVFLEQMNKAYSKHMLSDSLSDSVP

SEQ ID NO: 59 - protein, 361 aa, Drosophila
IQVPEELKEVLLQFSISFLVEQPPDVIDYAVEYFTKLQSERPSVSHTDQSTDDQLSVNSQDADAEPPVMASSRRKSVFAEAYDPEADDDDDGATAVFPKTDEQRARLVESVKNVLLFRSLEKEQMNQVLDAMFERKVQPGDFIIRQGDDGDNFYVIESGVYKVYINDKHINTYNHTGLFGELALLYNMPRAATVQAETSGLLWAMDRQTFRRILLKSAFRKRKMYEELLNSVPMLKALQNYERMNLADALVSKSYDNGERIIKQGDAADGMYFIEEGTVSVRMDQDDAEVEISQLGKGQYFGELALVTHRPRAASVYATGGVVKLAFLDVKAFERLLGPCMDIMKRNIDDYESQLVKIFGS

SEQ ID NO: 60 - protein, 392 aa, Homo sapiens
IQIPPGLTELLQGYTVEVLRQQPPDLVEFAVEYFTRLREARAPASVLPAATPRQSLGHPPPEPGPDRVADAKGDSESEEDEDLEVPVPSRFNRRVSVCAETYNPDEEEEDTDPRVIHPKTDEQRCRLQEACKDILLFKNLDQEQLSQVLDAMFERIVKADEHVIDQGDDGDNFYVIERGTYDILVTKDNQTRSVGQYDNRGSFGELALMYNTPRAATIVATSEGSLWGLDRVTFRRIIVKNNAKKRKMFESFIESVPLLKSLEVSERMKIVDVIGEKIYKDGERIITQGEKADSFYIIESGEVSILIRSRTKSNKDGGNQEVEIARCHKGQYFGELALVTNKPRAASAYAVGDVKCLVMDVQAFERLLGPCMDIMKRNISHYEEQLVKMFGS

SEQ ID NO: 61 - protein, 367 aa, Drosophila
IQVPEELKEVLLQFSISFLVEQPPDVIDYAVEYFTKLQSERPSVSHTDQSTDDQLSVNSQDADAEPPVMASSRRKSVFAEAYDPEADDDDDGATAVFPKTDEQRARLVESVKNVLLFRSLEKEQMNQVLDAMFERKVQPGDFIIRQGDDGDNFYVIESGVYKVYINDKHINTYNHTGLFGELALLYNMPRAATVQAETSGLLWAMDRQTFRRILLKSAFRKRKMYEELLNSVPMLKALQNYERMNLADALVSKSYDNGERIIKQGDAADGMYFIEEGTVSVRMDQDDAEVEISQLGKGQYFGELALVTHRPRAASVYATGGVVKLAFLDVKAFERLLGPCMDIMKRNIDDYESQLVKIFGSKNNITD

SEQ ID NO: 62 - protein, 413 aa, Homo sapiens
IEIPAGLTELLQGFTVEVLRHQPADLLEFALQHFTRLQQENERKGTARFGHEGRTWGDLGAAAGGGTPSKGVNFAEEPMQSDSEDGEEEEAAPADAGAFNAPVINRFTRRASVCAEAYNPDEEEDDAESRIIHPKTDDQRNRLQEACKDILLFKNLDPEQMSQVLDAMFEKLVKDGEHVIDQGDDGDNFYVIDRGTFDIYVKCDGVGRCVGNYDNRGSFGELALMYNTPRAATITATSPGALWGLDRVTFRRIIVKNNAKKRKMYESFIESLPFLKSLEFSERLKVVDVIGTKVYNDGEQIIAQGDSADSFFIVESGEVKITMKRKGKSEVEENGAVEIARCSRGQYFGELALVTNKPRAASAHAIGTVKCLAMDVQAFERLLGPCMEIMKRNIATYEEQLVALFGTNMDIVE

SEQ ID NO: 63 - protein, 304 aa, Mus musculus
AVCAEAYNPDEEEDDAESRIIHPKTDDQRNRLQEACKDILLFKNLDPEQMSQVLDAMFEKLVKEGEHVIDQGDDGDNFYVIDRGTFDIYVKCDGVGRCVGNYDNRGSFGELALMYNTPRAATITATSPGALWGLDRVTFRRIIVKNNAKKRKMYESFIESLPFLKSLEVSERLKVVDVIGTKVYNDGEQIIAQGDLADSFFIVESGEVKITMKRKGKSEVEENGAVEIARCFRGQYFGELALVTNKPRAASAHAITVKCLAMDVQAFERLLGPCMEIMKRNIATYEEQLVALFGTNMDIVEPTA

SEQ ID NO: 64 - protein, 401 aa, Mus musculus
MSHIQIPPGLTELLQGYTVEVGQQPPDLVDFAVEYFTRLREARRQESDTFIVSPTTFHTQESSAVPVIEEDGESDSDSEDADLEVPVPSKFTRRVSVCAETFNPDEEEEDNDPRVVHPKTDEQRCRLQEACKDILLFKNLDQEQLSQVLDAMFEKIVKTDEHVIDQGDDGDNFYVIERGTYDILVTKDNQTRSVGQYDNRGSFGELALMYNTPRAATIIATSEGSLWGLDRVTFRRIIVKNNAKKRKMFESFIESVPLFKSLEMSERMKIVDVIGEKIYKDGERIIAQGEKADSFYIIESGEVSILIRSKTKSNKNGGNQEVEIAHCHKGQYFGELALVTNKPRAASAYGVGDVKCLVMDVQAFERLLGPCMDIMKRNISHYEEQLVKMFGSNLDLMDPGQ

SEQ ID NO: 65 - protein, 377 aa, Drosophila
MSSDSSRRIQVPEELKEVLLQFSISFLVEQPPDVIDYAVEYFTKLQSERPSVSHTDQSTDDQLSVNSQDADAEPPVMASSRRKSVFAEAYDPEADDDDDGATAVFPKTDEQRARLVESVKNVLLFRSLEKEQMNQVLDAMFERKVQPGDFIIRQGDDGDNFYVIESGVYKVYINDKHINTYNHTGLFGELALLYNMPRAATVQAETSGLLWAMDRQTFRRILLKSAFRKRKMYEELLNSVPMLKALQNYERMNLADALVSKSYDNGERIIKQGDAADGMYFIEEGTVSVRMDQDDAEVEISQLGKGQYFGELALVTHRPRAASVYATGGVVKLAFLDVKAFERLLGPCMDIMKRNIDDYESQLVKIFGSKNNITDTR

SEQ ID NO: 66 - protein, 255 aa, Drosophila
FKRADRSRDGILSIQELGQYINRRIVEHIDEAIMNNAREFRRVDIGPADGLITWDEYHRFFLREHGMTEADIDEHDEIRHTALNRRAREDMMRDKARWSEAARTDLFTLTIDEYLSFRHPESSVSNLLELVDDLLRQFDQDGDDQLTLEEFSDLNVDDDDDLMRKSLISKTLVERREEFKRIIDKNHDGKADRGELLNYVNPKTPRYALQEAATLFSLCDENKDELLTLKEMTDHAEIFLQSKMIDTANSFHTEF

SEQ ID NO: 67 - protein, 256 aa, Homo sapiens
FSKVDVNTDRKISAKEMQRWIMEKTAEHFQEAMEESKTHFRAVDPDGDGHVSWDEYKVKFLASKGHSEKEVADAIRLNEELKVDEETQEVLENLKDRWYQADSPPADLLLTEEEFLSFLHPEHSRGMLRFMVKEIVRDLDQDGDKQLSVPEFISLPVGTVENQQGQDIDDNWVKDRKKEFEELIDSNHDGIVTAEELESYMDPMNEYNALNEAKQMIAVADENQNHHLEPEEVLKYSEFFTGSKLVDYARSVHEEF

SEQ ID NO: 68 - protein, 418 aa, Drosophila
MTFVNKLSKSFRWLRCSTLCAILFYMFFIGLILHSTSQKLQQQLKEQGTSSSSSSSSSSSSSSSSSSSSSTQADTKMAPNVGHNGAQRWPNTLQELEQQQQKHEQSMRLQPVANNLDSRNENNHRQPHPQSQKVQNASKSPNQPETVILAASNVNEKQILAKAFKRADRSRDGILSIQELGQYINRRIVEHIDEAIMNNAREFRRVDIGPADGLITWDEYHRFFLREHGMTEADIDEHDEIRHTALNRRAREDMMRDKARWSEAARTDLFTLTIDEYLSFRHPESSVSNLLELVDDLLRQFDQDGDDQLTLEEFSDLNVDDDDDLMRKSLISKTLVERREEFKRIIDKNHDGKADRGELLNYVNPKTPRYALQEAATLFSLCDENKDELLTLKEMTDHAEIFLQSKMIDTANSFHTEF

SEQ ID NO: 69 - protein, 215 aa, Drosophila
KGRSKSSQEWLTRQLADPYVEKARMMNYRCRSAFKLLEIDDKYGILRPGDTVLECGAAPGSWTQVAVERTNANGKQERAPQGAVFSIDLLHFHAVPGATIFGGMDFTSSLAQKRLREALQDRKVNCVLSDMAPNATGVRMLDQESITNLCYEVLRFALAMSAPQAHLVVKVWDNGDVPKLERDMLRFYEKVKRVKPRASRGDSAEHFLVARNFKG

SEQ ID NO: 70 - protein, 215 aa, Homo sapiens
KNRTGAEHLWLTRHLRDPFVKAAKVESYRCRSAFKLLEVNERHQILRPGLRVLDCGAAPGAWSQVAVQKVNAAGTDPSSPVGFVLGVDLLHIFPLEGATFLCPADVTDPRTSQRILEVLPGRRADVILSDMAPNATGFRDLDHDRLISLCLTLLSVTPDILQPGGTFLCKTWAGSQSRRLQRRLTEEFQNVRIIKPEASRKESSEVYFLATQYHG

SEQ ID NO: 71 - protein, 246 aa, Mus musculus
MAGHLKLVGVPLKVRRLHTAVCHYRGRTGAEHLWLTRHLKDPFVKAAKVESYRCRSAFKLLEMNEKHQILRPGLRVLDCGAAPGAWSQVAVQRVNATGADSSSPVGFVLGVDLLHIFPLAGATFLCPADVTDPRTFQKILELLPSRRADVILSDMAPNATGIRDLDHDKLISLCLTLVDMAVDILHPGGTLLCKTWAGSKSHLLQKRLTQEFQSTRVVKPEASRKESSEVYLLATQYRGGKGTRRP

SEQ ID NO: 72 - protein, 250 aa, Drosophila
MRLVFTGNCVFKRLLHTEIGGKYAKQQPRNLKGRSKSSQEWLTRQLADPYVEKARMMNYRCRSAFKLLEIDDKYGILRPGDTVLECGAAPGSWTQVAVERTNANGKQERAPQGAVFSIDLLHFHAVPGATIFGGMDFTSSLAQKRLREALQDRKVNCVLSDMAPNATGVRMLDQESITNLCYEVLRFALAMSAPQAHLVVKVWDNGDVPKLERDMLRFYEKVKRVKPRASRGDSAEHFLVARNFKGATDS

SEQ ID NO: 73 - protein, 199 aa, Drosophila
MERESNPMQPMCRSGCGFYGNPATDGLCSVCYKDSLRKKQQPPVSSTPVSVPSPQPSPTFSPAIAITNTAQPTVTSLQQPHNDVKEKITEEAAAAAKVNSEAITSATGPNTSTQAAASANEEDDKDKEDDKDAKKKKNRCGECRKKVGLTGFQCRCGGLYCAVHRYSDKHNCTFDYREHGAQEIRRNNPVVVGEKIQKI

SEQ ID NO: 74 - protein, 208 aa, Homo sapiens; identical to SEQ ID NO: 25

(19) United States
(12) Patent Application Publication
(10) Pub. No.: US 2005/0119206 A1
(43) Pub. Date: Jun. 2, 2005

(54) Cg8327, cg10823, cg18418, cg15862, cg3768, cg11447 and cg16750 homologous proteins involved in the regulation of energy homeostasis

(75) Inventors: Karsten Eulenberg, Bovenden (DE); Arnd Steuernagel, Gottingen (DE); Thomas Hader, Gottingen (DE); Gunter Bronner, Gottingen (DE)

Correspondence Address: ROTHWELL, FIGG, ERNST & MANBECK, P.C., 1425 K STREET, N.W., SUITE 800, WASHINGTON, DC, 20005, US

(73) Assignee: DeveloGen Aktiengesellschaft fuerentwicklungsbolog, Goettingen (DE)

(21) Appl. No.: 10/507,617

(22) PCT Filed: Mar. 14, 2003
(86) PCT No.: PCT/EP03/02714

(30) Foreign Application Priority Data
Mar. 14, 2002 (EP) 02005882.2
Mar. 15, 2002 (EP) 02006012.5
Mar. 20, 2002 (EP) 02006271.7
Mar. 25, 2002 (EP) 02006810.2

(51) Int. Cl. C12Q 1/68; C07H 21/04; A61K 48/00

(52) U.S. Cl. 514/44; 435/6; 435/69.1; 435/320.1; 435/325; 435/194; 530/388.26; 536/23.2

(57) ABSTRACT

The present invention discloses proteins regulating the energy homeostasis and the metabolism of triglycerides, and polynucleotides, which identify and encode the proteins disclosed in this invention. The invention also relates to the use of these sequences in the diagnosis, study, prevention and treatment of metabolic diseases and disorders.

This invention relates to the use of nucleic acid sequences encoding Gadfly Accession Number CG8327, Gadfly Accession Number CG10823, Gadfly Accession Number CG18418, Pka-R2 (cAMP-dependent protein kinase R2; Gadfly Accession Number CG15862), Gadfly Accession Number CG31475 (also referred to as Gadfly Accession Numbers CG14281 and CG3768), Gadfly Accession Number CG11447, and/or Gadfly Accession Number CG16751 (also referred to as Gadfly Accession Numbers CG16750) homologous proteins, and the polypeptides encoded thereby and to the use thereof or effector molecules thereof in the diagnosis, study, prevention, and treatment of diseases and disorders related to body-weight regulation, for example, but not limited to, metabolic diseases such as obesity as well as related disorders such as eating disorder, cachexia, diabetes mellitus, hypertension, coronary heart disease, hypercholesterolemia, dyslipidemia, osteoarthritis, and gallstones.

There are several metabolic diseases of human and animal metabolism, eg., obesity and severe weight loss, that relate to energy imbalance where caloric intake versus energy expenditure is imbalanced. Obesity is one of the most prevalent metabolic disorders in the world. It is still a poorly. understood human disease that becomes more and more relevant for Western society. Obesity is defined as an excess of body fat, frequently resulting in a significant impairment of health. Cardiovascular risk factors like hypertension, high blood levels of triglycerides and fasting glucose as well as low blood levels of HDL cholesterol are often linked to obesity. This typical cluster of symptoms is commonly defined as “metabolic syndrome” (Reaven, 2002, Circulation 106(3): 286-8 reviewed). Hyperlipidemia and elevation of free fatty acids correlate clearly with the metabolic syndrome, which is defined as the linkage between several diseases, including obesity and insulin resistance. This often occurs in the same patients and is a major risk factor for development of Type 2 diabetes and cardiovascular disease. It was suggested that the control of lipid levels and glucose levels is required to treat Type 2 Diabetes, heart disease, and other occurances of Metabolic Syndrome (see, for example, Santomauro A. T. et al., (1999) Diabetes, 48(9):1836-1841 and McCook, 2002, JAMA 288:2709-2716).

Human obesity is strongly influenced by environmental and genetic factors, whereby the environmental influence is often a hurdle for the identification of (human) obesity genes. Obesity is influenced by genetic, metabolic, biochemical, psychological, and behavioral factors. As such, it is a complex disorder that must be addressed on several fronts to achieve lasting positive clinical outcome.

Obesity is not to be considered as a single disorder but a heterogeneous group of conditions with (potential) multiple causes. Obesity is also characterized by elevated fasting plasma insulin and an exaggerated insulin response to oral glucose intake (Koltermann, J. Clin. Invest 65, 1980, 1272-1284) and a clear involvement of obesity in type 2 diabetes mellitus can be confirmed (Kopelman, Nature 404, 2000, 635-643).

Insulin amongst other hormones plays a key role in the regulation of the fuel metabolism. High blood glucose levels stimulate the secretion of insulin by pancreatic beta-cells. Insulin leads to the storage of glycogen and triglycerides and to the synthesis of proteins. The entry of glucose into muscles and adipose cells is stimulated by insulin. In patients who suffer from diabetes mellitus either the amount of insulin produced by the pancreatic islet cells is to low (Diabetes Type 1 or insulin dependent diabetes mellitus IDDM) or liver and muscle cells loose their ability to respond to normal blood insulin levels (insulin resistance). In the next stage pancreatic cells become unable to produce sufficient amounts of insulin (Diabetes Type II or non insulin dependent diabetes mellitus NIDDM).

Even if several candidate genes have been described which are supposed to influence the homeostatic system(s) that regulate body mass/weight, like leptin, VCPI, VCPL, or the peroxisome proliferator-activated receptor-gamma co-activator, the distinct molecular mechanisms and/or molecules influencing obesity or body weight/body mass regulations are not known.

Therefore, the technical problem underlying the present invention was to provide for means and methods for modulating (pathological) metabolic conditions influencing body-weight regulation and/or energy homeostatic circuits. The solution to said technical problem is achieved by providing the embodiments characterized in the claims.

Accordingly, the present invention relates to genes with novel functions in body-weight regulation, energy homeostasis, metabolism, and obesity. The present invention discloses a specific gene involved in the regulation of body-weight, energy homeostasis, metabolism, and obesity, and thus in disorders related thereto such as metabolic syndrome, eating disorder, cachexia, diabetes mellitus, hypertension, coronary heart disease, hypercholesterolemia, dyslipidemia, osteoarthritis, and gallstones. The present invention describes the human homologs of the Drosophila Gadfly Accession Number CG8327, Gadfly Accession Number CG10823, Gadfly Accession Number CG18418, Pka-R2 (cAMP-dependent protein kinase R2; Gadfly Accession Number CG15862), Gadfly Accession Number CG31475, Gadfly Accession Number CG11447, and/or Gadfly Accession Number CG16751 genes as being involved in those conditions mentioned above.

Polyamines (putrescine, spermidine, and spermine) can modulate the functions of RNA, DNA, nucleotide triphosphates, proteins, and other acidic substances (Igarashi K. and Kashiwagi K., 2000, Biochem Biophys Res Commun 271(3):559-564). Polyamines stimulate the activity of glycogen synthase (casein) kinase-1 from bovine kidney and different rat tissues (Singh T. J., 1988, Arch Biochem Biophys 267(1):167-175). The biosynthesis of polyamines, which are universally essential for cellular functions, takes place from arginine and methionine and involves 4 distinct enzymes: spermidine synthase, ornithine decarboxylase, S-adenosyl-L-methionine decarboxylase, and spermine synthase (Janne J. et al., 1991, Ann Med 23(3):241-259). Wahifors et al. (1990, DNA Cell Biol. 9:103-110) cloned a cDNA coding for the full-length subunit of human spermidine synthase. Myohanen et al. (1991, DNA Cell Biol. 10(6):467-474) isolated the human gene from a genomic library and mapped it to chromosome 1. A transgenic mouse line over-expresses the human spermidine synthase gene. The elevated spermidine synthase activity has no effect on tissue putrescine, spermidine or spermine levels (Kauppinen L. et al., 1993, Biochem J 293 (Pt 2):513-516).

Neuropeptide FF (NPFF) is a mammalian neuropeptide with multiple functions. NPFF might have an effect on memory and autonomic regulation, and might be involved in the hypothalamo-pituitary pathway of neuroendocrine regulation. The NPFF receptor appears to be coupled to a G-protein (Panula P. et al., 1996, Prog Neurobiol 48(4-5):461-487). The Neuropeptide FF receptor might be the receptor for Melanocortin to modulate cardiovascular regulation (Versteeg D. H. et al., 1998, Eur J Pharmacol 360(1):1-14). Neuropeptide FF (NPFF) is involved in pain modulation and opioid tolerance. This peptide acts through G protein-coupled receptors (GPCR). NPFF is part of a neurotransmitter system and together with neuropeptide AF involved in pain modulation and opinoid tolerance (Kotani, M. et al., 2001, Br J Pharmacol 133(1):138-144). The anti-opiate neuropeptides FF and AF (NPFF and NPAF) are involved in pain modulation as well as in opioid tolerance and may play a critical role in this process. The neuropeptide FF receptor is expressed in brain regions associated with opiate activity, and in adipose tissue (Elshourbagy N.A., 2000, J Biol Chem 275(34):25965-25971).

Energy transduction in mitochondria requires the transport of many specific metabolites across the inner membrane of this eukaryotic organelle. The mitochondrial carrier family (MCF) consists of at least thirty-seven proteins. (Kuan J. and Saier M. H., 1993, Crit Rev Biochem Mol Biol 28(3):209-233). The ADP/ATP, phosphate, and oxoglutarate/malate carrier proteins found in the inner membranes of mitochondria, and the uncoupling protein from mitochondria in mammalian brown adipose tissue, belong to the same protein superfamily (Walker J E, Runswick M J., 1993, J Bioenerg Biomembr 25(5):435-446). It was shown that overexpression of the human 2-oxoglutarate carrier lowers mitochondrial membrane potential in HEK-293 cells, signaling a uncoupling activity for the 2-oxoglutarate carrier (Yu X.X. et al., 2001, Biochem J 353(Pt 2):369-375). Pyridoxal 5′-phosphate and some other lysine reagents inactivate the purified, reconstituted mitochondrial oxoglutarate transport protein. Glutarate and substrates of other mitochondrial carrier proteins were unable to protect the carrier. Pyridoxal 5′-phosphate interacts with the oxoglutarate carrier at a site(s) (i.e., a lysine residue(s) and/or the amino-terminal glycine residue) which is essential for substrate translocation and may be localized at or near the substrate-binding site (Natuzzi D. et al., 1999, J Bioenerg Biomembr 31(6):535-541).

The Drosophila Pka-R2 encodes for a cAMP-dependent protein kinase regulator protein that is involved in circadian rhythm and protein amino acid phosphorylation. Two types of cAMP-dependent protein kinase (PKA), type I and type II, are present in Drosophila heads, and type II PKA was found to be a major isozyme. The regulatory subunit of type II PKA (RII) may play an essential role in the regulation of neuronal activity in the brain of Drosophila, and possibly in human (Inoue H. and Yoshioka T., 1997, Biochem Biophys Res Commun 235(1):223-226).

cAMP-dependent protein kinase (PKA) is an essential enzyme in the signaling pathway of the second messenger cAMP. Through phosphorylation of target proteins, PKA controls many biochemical events in the cell including regulation of metabolism, ion transport, and gene transcription. The holoenzyme of PKA, a tetramer consisting of 2 regulatory and 2 catalytic subunits, is inactive in the absence of cAMP. Activation occurs when 2 cAMP molecules bind to each regulatory subunit, eliciting a reversible conformational change that releases active catalytic subunits. Four distinct regulatory subunits have been identified: RI-alpha, RI-beta, RII-alpha, and RII-beta. Distinct tissue-specific expression patterns of each of the 4 isoforms have been found. The evolution and conservation of the 4 regulatory subunits, each encoded by a separate gene, may have been necessary to accomplish such diverse functions as regulation of intermediary metabolism and neuronal functions including learning and gene transcription in the nucleus.

The major regulator of lipolysis in white adipocytes and brown adipocytes is cAMP and the actions of cAMP are mediated by protein kinase A (PKA). Multiple subunits of PKA, including RII beta, R1 alpha, C alpha, and C beta 1, are expressed in fat cells but the major holoenzyme assembled under normal conditions contains RII beta and C alpha. Targeted disruption of the RII beta gene in mice revealed that both white and brown adipocytes are capable of compensating by increasing the level of RI alpha. Nevertheless, the mice display a lean phenotype, have an elevated metabolic rate due to activation and induction of uncoupling protein in brown fat, and are resistant to diet-induced obesity and insulin resistance (McKnight G. S., 1998, Recent Prog Horm Res 53:139-159).

The CREC family is a family of multiple EF-hand, low-affinity Ca(2+)-binding proteins. It consists of a number of proteins that localize to the secretory pathway of mammalian cells, including the protein of the invention, Cab45. Similar proteins are found in quite diverse invertebrate organisms. The proteins may participate in Ca(2+)-regulated activities. Some CREC family members are involved in pathological activities such as malignant cell transformation, mediation of the toxic effects of snake venom toxins and putative participation in amyloid formation (Honore B. and Vorum H., 2000, FEBS Lett 466(1):11-18). Cab45 is ubiquitously expressed, contains an NH₂ terminal signal sequence but no membrane-anchor sequences, and binds Ca²⁺ due to the presence of six EF-hand motifs. Within the superfamily of calcium-binding proteins, it belongs to a recently identified group of proteins consisting of Reticulocalbin and ERC 55, both of which share significant sequence homology with Cab45 outside the EF-hand motifs. Cab45 is a soluble protein resident in the Golgi lumen (Scherer P. E., 1996, J Cell Biol 133(2):257-268).

Ribosomal RNAs undergo several nucleotide modifications including methylation. FtsJ is an Escherichia coli methyltransferase of the 23 S rRNA. The methylated nucleotide is 2′-O-methyluridine 2552. (Caldas T. et al., 2000, J Biol Chem 275(22):16414-16419). The cell division protein FtsJ is well conserved, from bacteria to humans (Bugl H. et al., 2000, Mol Cell 6(2):349-360). Cellular RNAs in eukaryotes undergo extensive posttranscriptional modifications. FTSJ2 (FtsJ homolog 2), a human gene encoding a RNA methyltransferase, belongs to a family of evolutionarily conserved S-adenosylmethionine-binding proteins. FTSJ2 transcripts are abundant in skeletal muscle, placenta, and heart, as well as in cancer cells. FTSJ2 is a nucleolar RNA methyltransferase involved in eukaryotic RNA processing and modification (Ching Y. P. et al., 2002, Genomics 79(1):2-6).

AWP1 is a ubiquitously expressed protein that associates with serine/threonine kinase PRK1 in vivo (Duan W. et al., 2000, Gene 256(1-2):113-121). The Awp1 gene is switched on during early human and mouse development. AWP1 possesses a conserved zf-A20 zinc finger domain at its amino-terminal domain and a zf-AN1 zinc finger domain at the carboxy-terminus. AWP1 may play a regulatory role in mammalian signal transduction pathways. PRK1 is a serine/threonine kinase that has been shown to be activated by RhoA (Amano M. et al., 1996, Science 271, 648-650). The serine/threonine kinase Prk1p is a factor regulating the actin cytoskeleton organization in yeast (Zeng G and Cai M., 1999, J Cell Biol 144(1):71-82).

So far, it has not been described that Gadfly Accession Number CG8327, Gadfly Accession Number CG10823, Gadfly Accession Number CG18418, Pka-R2 (cAMP-dependent protein kinase R2; Gadfly Accession Number CG15862), Gadfly Accession Number CG31475, Gadfly Accession Number CG1447, and/or Gadfly Accession Number CG16751 encoded proteins and closely related proteins, particularly human proteins spermidine synthase (SRM), neuropeptide FF 2 (NPFF2; also referred to as neuropeptide G protein-coupled receptor; G protein-coupled receptor 74; GPR74), neuropeptide FF 1 (NPFF1; also referred to as RFamide-related peptide receptor), orexin receptor 1 (OX1R), orexin receptor 2 (OX2R), solute carrier family 25 (mitochondrial carrier; oxoglutarate carrier) member 11 (SLC25A11), protein kinase, cAMP-dependent, regulatory, type II, alpha (PRKAR2A), protein kinase, cAMP-dependent, regulatory, type II, beta (PRKAR2B), calcium binding protein Cab45 precursor (Cab45; also referred to as stromal cell derived factor 4), cell division protein FtsJ (FtsJ2), protein associated with PKR1 (AWP1), and/or zinc finger protein 216 (ZNF216) are involved in the regulation of energy homeostasis and body-weight regulation and related disorders, and thus, no functions in metabolic diseases and other diseases as listed above have been discussed.

In this invention we demonstrate that the correct gene dose of Gadfly Accession Number CG8327, Gadfly Accession Number CG10823, Gadfly Accession Number CG18418, Pka-R2 (cAMP-dependent protein kinase R2; Gadfly Accession Number CG15862), Gadfly Accession Number CG31475, Gadfly Accession Number CG11447, and/or Gadfly Accession Number CG16751 is essential for maintenance of energy homeostasis. A genetic screen was used to identify that mutation of Gadfly Accession Number CG8327, Gadfly Accession Number CG10823, Gadfly Accession Number CG18418, Pka-R2 (cAMP-dependent protein kinase R2; Gadfly Accession Number CG15862), Gadfly Accession Number CG31475, Gadfly Accession Number CG11447, and/or Gadfly Accession Number CG16751 homologous genes cause obesity, reflected by a significant increase of triglyceride content, the major energy storage substance.

Polynucleotides encoding proteins with homologies to Gadfly Accession Number CG8327, Gadfly Accession Number CG10823, Gadfly Accession Number CG18418, Pka-R2 (cAMP-dependent protein kinase R2; Gadfly Accession Number CG15862), Gadfly Accession Number CG31475, Gadfly Accession Number CG11447, and/or Gadfly Accession Number CG16751 are suitable to investigate diseases and disorders as described above. Further new compositions useful in diagnosis, treatment, and prognosis of diseases and disorders as described above are provided.

Before the present proteins, nucleotide sequences, and methods are described, it is understood that this invention is not limited to the particular methodology, protocols, cell lines, vectors, and reagents described as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the cell lines, vectors, and methodologies that are reported in the publications which might be used in connection with the invention. Nothing herein is to be construed as an admission that the invention is not entitled to antedate such disclosure.

The present invention discloses that Gadfly Accession Number CG8327, Gadfly Accession Number CG10823, Gadfly Accession Number CG18418, Pka-R2 (cAMP-dependent protein kinase R2; Gadfly Accession Number CG15862), Gadfly Accession Number CG31475, Gadfly Accession Number CG11447, and/or Gadfly Accession Number CG16751 homologous proteins are regulating the energy homeostasis and fat metabolism especially the metabolism and storage of triglycerides, and polynucleotides, which identify and encode the proteins disclosed in this invention. The invention also relates to vectors, host cells, antibodies, and recombinant methods for producing the polypeptides and polynucleotides of the invention. The invention also relates to the use of these sequences in the diagnosis, study, prevention, and treatment of diseases and disorders, for example, but not limited to, metabolic diseases such as obesity as well as related disorders such as metabolic syndrome, eating disorder, cachexia, diabetes mellitus, hypertension, coronary heart disease, hypercholesterolemia, dyslipidemia, osteoarthritis, and gallstones.

The term “polynucleotide comprising the nucleotide sequence as shown in GenBank Accession number” relates to the expressible gene of the nucleotide sequences deposited under the corresponding GenBank Accession number. The term “GenBank Accession Number” relates to NCBI GenBank database entries (Ref.: Benson et al., (2000) Nucleic Acids Res. 28: 15-18).

Gadfly Accession Number CG8327, Gadfly Accession Number CG10823, Gadfly Accession Number CG18418, Pka-R2 (cAMP-dependent protein kinase R2; Gadfly Accession Number CG15862), Gadfly Accession Number CG31475, Gadfly Accession Number CG11447, and/or Gadfly Accession Number CG16751 homologous proteins and nucleic acid molecules coding therefore are obtainable from insect or vertebrate species, e.g. mammals or birds. Particularly preferred are homologous nucleic acids, particularly nucleic acids encoding a human spermidine synthase (SRM), neuropeptide FF 2 (NPFF2), neuropeptide FF 1 (NPFF1), orexin receptor 1 (OX1R), orexin receptor 2 (OX2R), solute carrier family 25 (mitochondrial carrier; oxoglutarate carrier) member 11 (SLC25A11), protein kinase, cAMP-dependent, regulatory, type II, alpha (PRKAR2A), protein kinase, cAMP-dependent, regulatory, type II, beta (PRKAR2B), calcium binding protein Cab45 precursor (Cab45), cell division protein FtsJ (FtsJ2), protein associated with PKR1 (AWP1), or zinc finger protein 216 (ZNF216).

The invention particularly relates to a nucleic acid molecule encoding a polypeptide contributing to regulating the energy homeostasis and the metabolism of triglycerides, wherein said nucleic acid molecule comprises
(a) the nucleotide sequence of or a nucleotide sequence encoding (i) Gadfly Accession Number CG8327, human spermidine synthase (SEQ ID NO: 1; GenBank Accession Number NM_{—}003132), (ii) Gadfly Accession Number CG10823, neuropeptide FF 2 (SEQ ID NO: 4; GenBank Accession Number NM_{—}053036), neuropeptide FF 1 (SEQ ID NO: 6; GenBank Accession Number NM_{—}022146), orexin receptor 1 (SEQ ID NO: 8; GenBank Accession Number NM_{—}001525), orexin receptor 2 (SEQ ID NO: 10; GenBank Accession Number NM_{—}001526), (iii) Gadfly Accession Number CG18418, solute carrier family 25 (mitochondrial carrier;

oxoglutarate carrier) member 11 (SEQ ID NO: 12; GenBank Accession Number NM_{—}003562), (iv) Pka-R2 (cAMP-dependent protein kinase R2; Gadfly Accession Number CG15862), protein kinase, cAMP-dependent, regulatory, type II, alpha (SEQ ID NO: 14; GenBank Accession Number NM_{—}004157), protein kinase, cAMP-dependent, regulatory, type II, beta (SEQ ID NO: 16; GenBank Accession Number NM_{—}002736; formerly GenBank Accession Number XM_{—}004959), (v) Gadfly Accession Number CG31475, calcium binding protein Cab45 precursor variant 1 or variant 2 (SEQ ID Nos: 18 and 20; GenBank Accession Numbers NM_{—}016176 and NM_{—}016547), (vi) Gadfly Accession Number CG11447, cell division protein FtsJ (SEQ ID NO: 22; GenBank Accession Number NM_{—}013393), (vii) Gadfly Accession Number CG16751, protein associated with PKR1 (SEQ ID NO: 24; GenBank Accession Number NM_{—}019006; formerly GenBank Accession Number XM_{—}044547), and/or zinc finger protein 216 (SEQ ID NO: 26; GenBank Accession Number NM_{—}006007), and/or a sequence complementary thereto,
(b) a nucleotide sequence which hybridizes at 50° C. in a solution containing 1×SSC and 0.1% SDS to a sequence of (a),
(c) a sequence corresponding to the sequences of (a) or (b) within the degeneration of the genetic code,
(d) a sequence which encodes a polypeptide which is at least 85%, preferably at least 90%, more preferably at least 95%, more preferably at least 98% and up to 99.6% identical to the amino acid sequences of Gadfly Accession Number CG8327, Gadfly Accession Number CG10823, Gadfly Accession Number CG18418, Pka-R2 (cAMP-dependent protein kinase R2; Gadfly Accession Number CG15862), Gadfly Accession Number CG31475, Gadfly Accession Number CG11447, and/or Gadfly Accession Number CG16751,
(e) a sequence encoding a Gadfly Accession Number CG8327, Gadfly Accession Number CG10823, Gadfly Accession Number CG18418, Pka-R2 (cAMP-dependent protein kinase R2; Gadfly Accession Number CG15862), Gadfly Accession Number CG31475, Gadfly Accession Number CG11447, and/or Gadfly Accession Number CG16751 homologous protein, preferably a human Gadfly Accession Number CG8327, Gadfly Accession Number CG10823, Gadfly Accession Number CG18418, Pka-R2 (cAMP-dependent protein kinase R2; Gadfly Accession Number CG15862), Gadfly Accession Number CG31475, Gadfly Accession Number CG11447, and/or Gadfly Accession Number CG16751 homologous protein, particularly human spermidine synthase (SEQ ID NO: 2; GenBank Accession Number NP_{—}003123), neuropeptide FF 2 (SEQ ID NO: 5; GenBank Accession Number NP_{—}444264), neuropeptide FF 1 (SEQ ID NO: 7; GenBank Accession Number NP_{—}071429), orexin receptor 1 (SEQ ID NO: 9; GenBank Accession Number NP_{—}001516), orexin receptor 2 (SEQ ID NO: 11; GenBank Accession Number NP_{—}001517), solute carrier family 25 (mitochondrial carrier; oxoglutarate carrier) member 11 (SEQ ID NO: 13; GenBank Accession Number NP_{—}003553), protein kinase, cAMP-dependent, regulatory, type II, alpha (SEQ ID NO: 15; GenBank Accession Number NP_{—}004148), protein kinase, cAMP-dependent, regulatory, type II, beta (SEQ ID NO: 17; GenBank Accession Number NP_{—}002727), calcium binding protein Cab45 precursor variant 1 or variant 2 (SEQ ID Nos: 19 and 21; GenBank Accession Numbers NP_{—}057260 and NP_{—}057631), cell division protein FtsJ (SEQ ID NO: 23; GenBank Accession Number NP_{—}037525), protein associated with PKR1 (SEQ ID NO: 25; GenBank Accession Number NP_{—}061879), and/or zinc finger protein 216 (SEQ ID NO: 27; GenBank Accession Number NP_{—}005998),
(f) a sequence which differs from the nucleic acid molecule of (a) to (d) by mutation and wherein said mutation causes an alteration, deletion, duplication and/or premature stop in the encoded polypeptide or
(g) a partial sequence of any of the nucleotide sequences of (a) to (e) having a length of at least 15 bases, preferably at least 20 bases, more preferably at least 25 bases and most preferably at least 50 bases.

The invention is based on the finding that Gadfly Accession Number CG8327, Gadfly Accession Number CG10823, Gadfly Accession Number CG18418, Pka-R2 (cAMP-dependent protein kinase R2; Gadfly Accession Number CG15862), Gadfly Accession Number CG31475, Gadfly Accession Number CG11447, and/or Gadfly Accession Number CG16751 homologous proteins (herein referred to as SRM, NPFF2, NPFF1, OX1R, OX2R, SLC25A11, PRKAR2A, PRKAR2B, Cab45, FtsJ2, AWP1, or ZNF216, or as the proteins of the invention) and the polynucleotides encoding these, are involved in the regulation of triglyceride storage and therefore energy homeostasis. The invention describes the use of these polypeptides, polynucleotides and effectors thereof for the diagnosis, study, prevention, or treatment of diseases and disorders related thereto, including metabolic diseases such as obesity as well as related disorders such as metabolic syndrome, eating disorder, cachexia, diabetes mellitus, hypertension, coronary heart disease, hypercholesterolemia, dyslipidemia, osteoarthritis, or gallstones.

Accordingly, the present invention relates to genes with novel functions in body-weight regulation, energy homeostasis, metabolism, and obesity, fragments of said genes, polypeptides encoded by said genes or fragments thereof, and effectors e.g. antibodies, biologically active nucleic acids, such as antisense molecules, RNAi molecules or ribozymes, aptamers, peptides or low-molecular weight organic compounds recognizing said polynucleotides or polypeptides.

The ability to manipulate and screen the genomes of model organisms such as the fly Drosophila melanogaster provides a powerful tool to analyze biological and biochemical processes that have direct relevance to more complex vertebrate organisms due to significant evolutionary conservation of genes, cellular processes, and pathways (see, for example, Adams M. D. et al., (2000) Science 287: 2185-2195). Identification of novel gene functions in model organisms can directly contribute to the elucidation of correlative pathways in mammals (humans) and of methods of modulating them. A correlation between a pathology model (such as changes in triglyceride levels as indication for metabolic syndrome including obesity) and the modified expression of a fly gene can identify the association of the human ortholog with the particular human disease.

In one embodiment, a forward genetic screen is performed in fly displaying a mutant phenotype due to misexpression of a known gene (see, Johnston Nat Rev Genet 3: 176-188 (2002); Rorth P., (1996) Proc Natl Acad Sci U S A 93: 12418-12422). Triglycerides are the most efficient storage for energy in cells. Obese people mainly show a significant increase in the content of triglycerides. In this invention, we have used a genetic screen to identify mutations of genes encoding the proteins of the invention and homologous genes that cause changes in the body weight which is reflected by a significant change of triglyceride levels. In order to isolate genes with a function in energy homeostasis, several thousand proprietary and publicly available EP-lines were tested for their triglyceride content after a prolonged feeding period (see Examples for more detail). Lines with significantly changed triglyceride content were selected as positive candidates for further analysis. The change of triglyceride content due to the loss of a gene function suggests gene activities in energy homeostasis in a dose dependent manner that control the amount of energy stored as triglycerides.

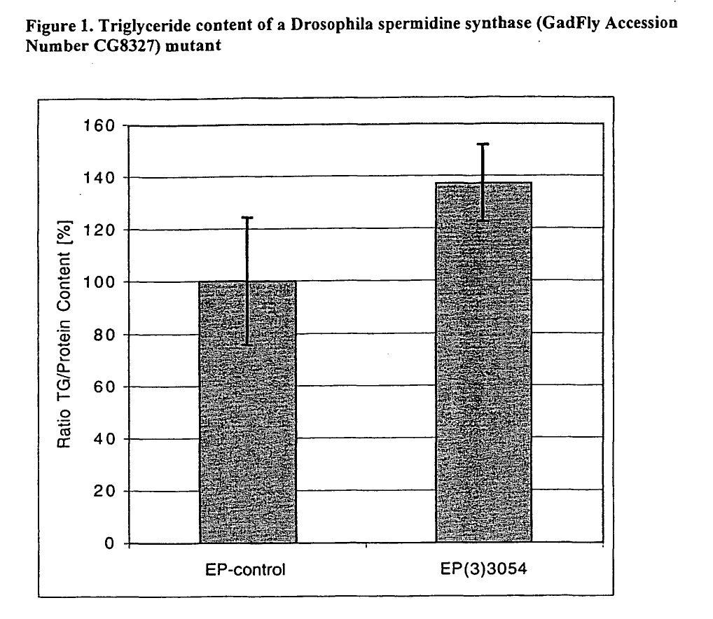

In one embodiment, flies homozygous for the integration of vectors for Drosophila lines EP(3)3054, HD-EP(3)31068, HD-EP(3)31149, EP(3)3322, EP(2)2162, EP(3)0650, HD-EP(3)31393, and HD-EP(3)32007 were analyzed in an assay measuring the triglyceride contents of these flies, illustrated in more detail in the EXAMPLES section of the invention. The results of the triglyceride content analysis are shown in FIGS. 1, 5, 9, 13, 17, 21, and 25.

The increase of triglyceride content due to the loss of a gene function suggests gene activities in energy homeostasis in a dose dependent manner that controls the amount of energy stored as triglycerides.

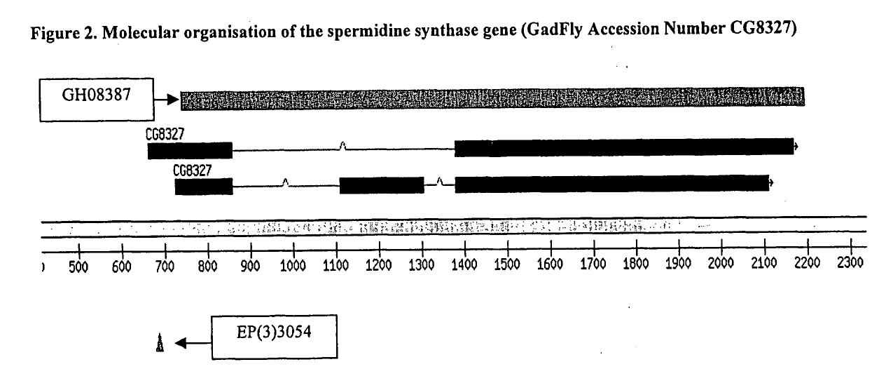

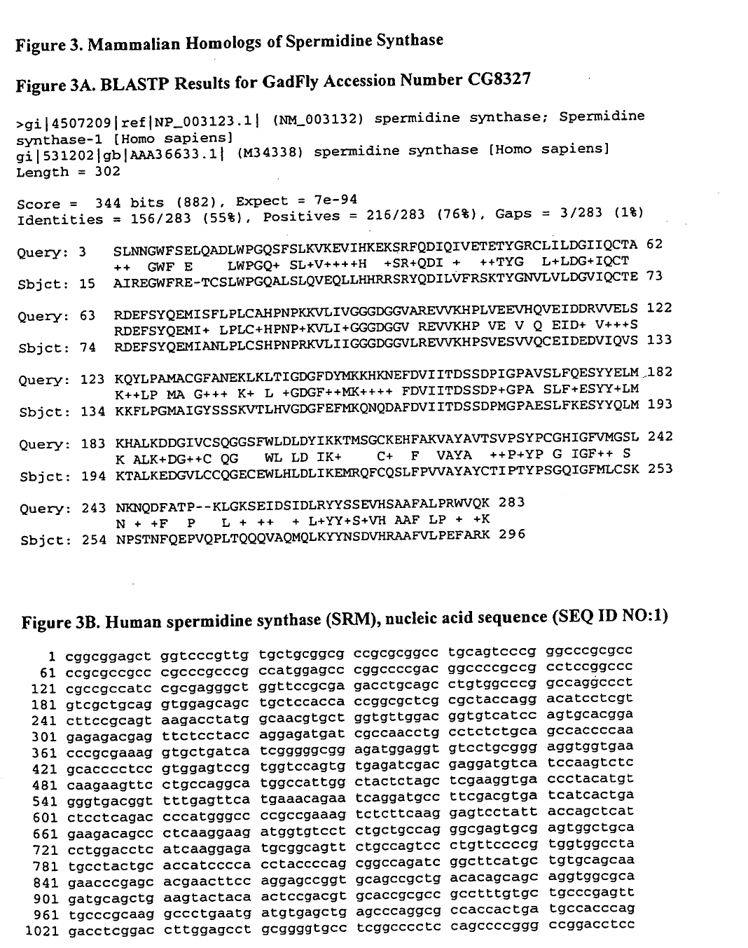

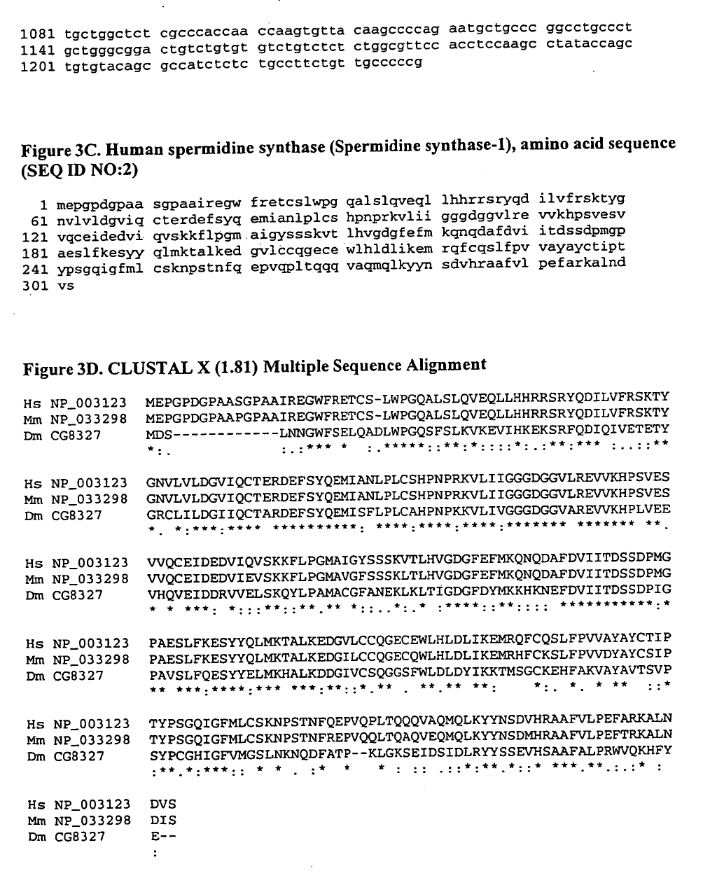

Nucleic acids-encoding the proteins of the present invention were identified using a plasmid-rescue technique. Genomic DNA sequences were isolated that are localized adjacent to the EP vector (herein EP(3)3054, HD-EP(3)31068, HD-EP(3)31149, EP(3)3322, EP(2)2162, EP(3)0650, HD-EP(3)31393, and HD-EP(3)32007) integration. Using those isolated genomic sequences public databases like Berkeley Drosophila Genome Project (GadFly; see also FlyBase (1999) Nucleic Acids Research 27:85-88) were screened thereby identifying the integration sites of the vectors, and the corresponding genes, described in more detail in the EXAMPLES section. The molecular organization of the genes is shown in FIGS. 2, 6, 10, 14, 18, 22, and 26. The Drosophila genes and proteins encoded thereby with functions in the regulation of triglyceride metabolism were further analysed in publicly available sequence databases (see EXAMPLES for more detail) and mammalian homologs were identified (see FIGS. 3, 7, 11, 15, 19, 23, and 27). Comparisons (Clustal analysis) between the proteins of different species (human, mouse and Drosophila) were conducted (see FIGS. 3, 7, 11, 15, 19, 23, and 27). Based upon homology, the Drosophila proteins of the invention and each homologous protein or peptide may share at least some activity. No functional data described the regulation of body weight control and related metabolic diseases such as obesity and diabetes are available in the prior art for the genes of the invention.

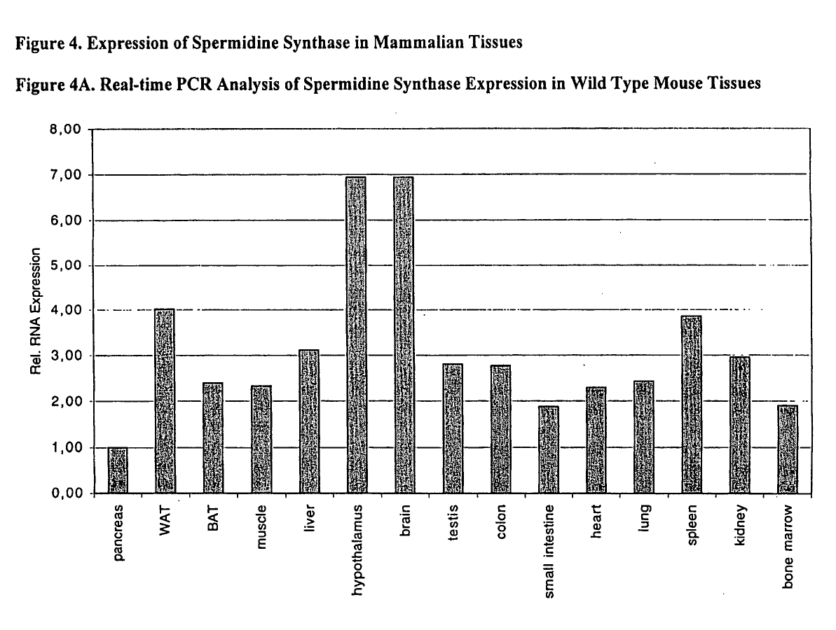

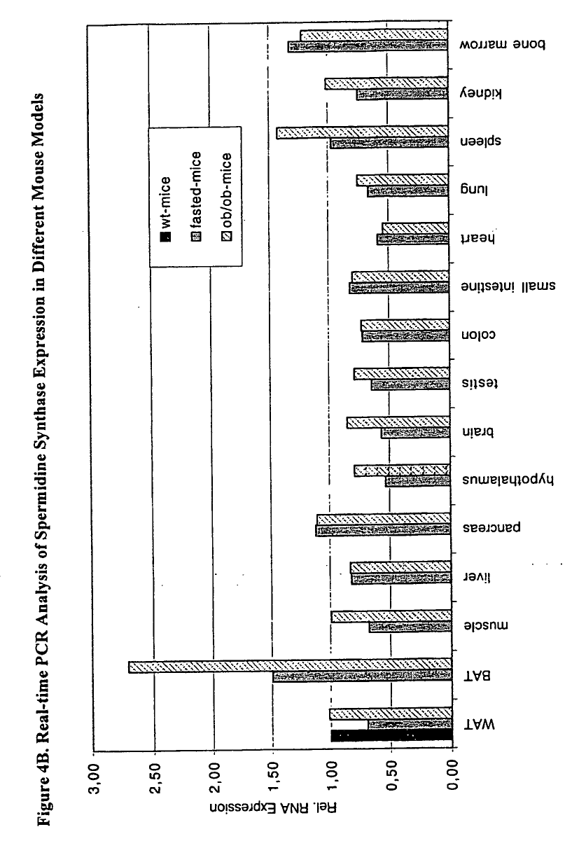

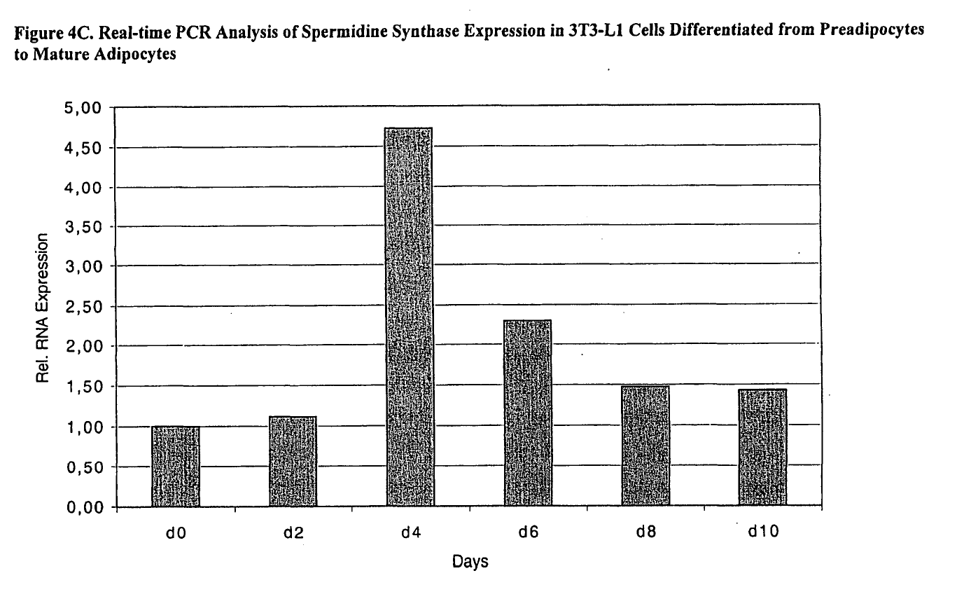

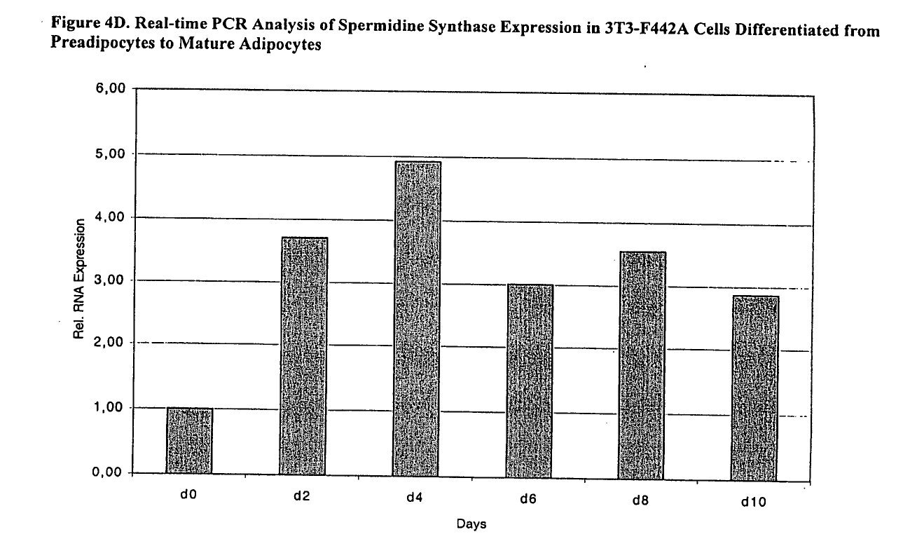

The function of the mammalian homologs in energy homeostasis was further validated in this invention by analyzing the expression of the transcripts in different tissues and by analyzing the role in adipocyte differentiation (see FIGS. 4, 8, 12, 16, 20, and 24). Expression profiling studies (see Examples for more detail) confirm the particular relevance of the proteins of the invention as regulators of enery metabolism in mammals. Further, we show that the proteins of the invention are regulated by fasting or by genetically induced obesity. In this invention, we used mouse models of insulin resistance and/or diabetes, such as mice carrying gene knockouts in the leptin pathway (for example, ob (leptin) or db (leptin receptor) mice) to study the expression of the proteins of the invention. Such mice develop typical symptoms of diabetes, show hepatic lipid accumulation, and frequently have increased plasma lipid levels (see Bruning et al, 1998, Mol. Cell. 2:449-569). In addition, we show in this invention that the mRNAs of the proteins of the invention are regulated during adipocyte differentiation in vitro (see EXAMPLES for more detail), suggesting a role as modulator of adipocyte lipid accumulation. Thus, we conclude that the protein of the invention (or variants thereof) have a function in the metabolism of mature mammalian adipocytes.

The present invention further describes polypeptides-comprising the amino acid sequences of SRM, NPFF2, NPFF1, OX1R, OX2R, SLC25A11, PRKAR2A, PRKAR2B, Cab45, FtsJ2, AWP1, ZNF216, and homologous proteins. Based upon homology, the proteins of the invention and each homologous protein or peptide may share at least some activity. No functional data described the regulation of body weight control and related metabolic diseases such as obesity are available in the prior art for the genes of the invention.

The invention also encompasses polynucleotides that encode a protein of the invention or homologous proteins. Accordingly, any nucleic acid sequence, which encodes the amino acid sequences of a protein of the invention or homologous proteins, can be used to generate recombinant molecules that express a protein of the invention or homologous proteins. It will be appreciated by those skilled in the art that as a result of the degeneracy of the genetic code, a multitude of nucleotide sequences encoding the proteins of the invention or homologous proteins, some bearing minimal homology to the nucleotide sequences of any known and naturally occurring gene, may be produced. Thus, the invention contemplates each and every possible variation of nucleotide sequence that can be made by selecting combinations based on possible codon choices.

Also encompassed by the invention are polynucleotide sequences that are capable of hybridizing to the claimed nucleotide sequences, and in particular, those of the polynucleotide encoding a protein of the invention, under various conditions of stringency. Hybridization conditions are based on the melting temperature (Tm) of the nucleic acid binding complex or probe, as taught in Wahl, G. M. and S. L. Berger (1987: Methods Enzymol. 152:399-407) and Kimmel, A. R. (1987; Methods Enzymol. 152:507-511), and may be used at a defined stringency. Preferably, hybridization under stringent conditions means that after washing for 1 h with 1×SSC and 0.1% SDS at 50° C., preferably at 55° C., more preferably at 62° C. and most preferably at 68° C., particularly for 1 h in 0.2×SSC and 0.1% SDS at 50° C., preferably at 55° C., more preferably at 62° C. and most preferably at 68° C., a positive hybridization signal is observed. Altered nucleic acid sequences encoding a protein of the invention or homologous proteins which are encompassed by the invention include deletions, insertions, or substitutions of different nucleotides resulting in a polynucleotide that encodes the same or a functionally equivalent protein.

The encoded proteins may also contain deletions, insertions, or substitutions of amino acid residues, which produce a silent change and result in functionally equivalent proteins. Deliberate amino acid substitutions may be made on the basis of similarity in polarity, charge, solubility, hydrophobicity, hydrophilicity, and/or the amphipathic nature of the residues as long as the biological activity of the protein is retained. Furthermore, the invention relates to peptide fragments of the proteins or derivates of such peptides such as cyclic peptides, retro-inverso peptides or peptide mimetics having a length of at least 4, preferably at least 6 and up to 50 amino acids.

Also included within the scope of the present invention are alleles of the genes encoding a protein of the invention or homologous proteins. As used herein, an ‘allele’ or ‘allelic sequence’ is an alternative form of the gene, which may result from at least one mutation in the nucleic acid sequence. Alleles may result in altered mRNAs or polypeptides whose structures or function may or may not be altered. Any given gene may have none, one, or many allelic forms. Common mutational changes, which give rise to alleles, are generally ascribed to natural deletions, additions, or substitutions of nucleotides. Each of these types of changes may occur alone, or in combination with the others, one or more times in a given sequence.

The nucleic acid sequences encoding a protein of the invention or homologous proteins may be extended utilizing a partial nucleotide sequence and employing various methods known in the art to detect upstream sequences such as promoters and regulatory elements. For example, one method which may be employed, “restriction-site” PCR, uses universal primers to retrieve unknown sequence adjacent to a known locus (Sarkar, G. (1993) PCR Methods Applic. 2:318-322). Inverse PCR may also be used to amplify or extend sequences using divergent primers based on a known region (Triglia, T. et al. (1988) Nucleic Acids Res. 16:8186). Another method which may be used is capture PCR which involves PCR amplification of DNA fragments adjacent to a known sequence in human and yeast artificial chromosome DNA (Lagerstrom, M. et al. (PCR Methods Applic. 1:111-119). Another method which may be used to retrieve unknown sequences is that of Parker, J. D. et al. (1 991; Nucleic Acids Res. 19:3055-3060). Additionally, one may use PCR, nested primers, and PROMOTERFINDER libraries to walk in genomic DNA (Clontech, Palo Alto, Calif.). This process avoids the need to screen libraries and is useful in finding intron/exon junctions.

In order to express a biologically active protein, the nucleotide sequences encoding the protein or functional equivalents may be inserted into appropriate expression vectors, i.e. a vector, which contains the necessary elements for the transcription and translation of the inserted coding sequence. Methods, which are well known to those skilled in the art, may be used to construct expression vectors containing sequences encoding the proteins and appropriate transcriptional and translational control elements. These methods include in vitro recombinant DNA techniques, synthetic techniques, and in vivo genetic recombination. Such techniques are described in Sambrook, J. et al. (1989) Molecular Cloning, A Laboratory Manual, Cold Spring Harbor Press, Plainview, N.Y., and Ausubel, F. M. et al. (1 989) Current Protocols in Molecular Biology, John Wiley & Sons, New York, N.Y.

A variety of expression vector/host systems may be utilized to contain and express sequences encoding a protein of the invention or homologous proteins. These include, but are not limited to, micro-organisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or PBR322 plasmids); or animal cell systems. The “control elements” or “regulatory sequences” are those non-translated regions of the vector-enhancers, promoters, 5′ and 3′ untranslated regions which interact with host cellular proteins to carry out transcription and translation. Such elements may vary in their strength and specificity. Depending on the vector system and host utilized, any number of suitable transcription and translation elements, including constitutive and inducible promoters, may be used.

The presence of polynucleotide sequences encoding a protein of the invention or homologous proteins can be detected by DNA-DNA or DNA-RNA hybridization and/or amplification using probes or portions or fragments of polynucleotides encoding the protein. Nucleic acid amplification-based assays involve the use of oligonucleotides or oligomers based on the sequences specific for a protein of the invention and nucleic acids to detect transformants containing DNA or RNA encoding the proteins. As used herein ‘oligonucleotides’ or ‘oligomers’ refer to a nucleic acid sequence of at least about 10 nucleotides and as many as about 60 nucleotides, preferably about 15 to 30 nucleotides, and more preferably about 20-25 nucleotides, which can be used as a probe or amplimer.

A variety of protocols for detecting and measuring the expression of the proteins of the invention or homologous proteins, using either polyclonal or monoclonal antibodies specific for the protein are known in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on the protein is preferred, but a competitive binding assay may be employed. These and other assays are described, among other places, in Hampton, R. et al. (1990; Serological Methods, a Laboratory Manual, APS Press, St Paul, Minn.) and Maddox, D. E. et al. (1983; J. Exp. Med. 158:1211-1216).

A wide variety of labels and conjugation techniques are known by those skilled in the art and may be used in various nucleic acid and amino acid assays. Means for producing labeled hybridization or PCR probes for detecting sequences related to polynucleotides encoding a protein of the invention or homologous proteins include oligo-labeling, nick translation, end-labeling or PCR amplification using a labeled nucleotide.

Alternatively, the sequences encoding a protein of the invention or homologous proteins, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits (Pharmacia & Upjohn, (Kalamazoo, Mich.); Promega (Madison Wis.); and U.S. Biochemical Corp., (Cleveland, Ohio).

Suitable reporter molecules or labels, which may be used, include radionuclides, enzymes, fluorescent, chemiluminescent, or chromogenic agents as well as substrates, co-factors, inhibitors, magnetic particles, and the like.

Host cells transformed with nucleotide sequences encoding a protein of the invention or homologous proteins may be cultured under conditions suitable for the expression and recovery of the protein from cell culture. The protein produced by a recombinant cell may be secreted or contained intracellularly depending on the sequence and/or the vector used. As will be understood by those of skill in the art, expression vectors containing polynucleotides which encode the protein may be designed to contain signal sequences, which direct secretion of the protein through a prokaryotic or eukaryotic cell membrane. Other recombinant constructions may be used to join sequences encoding the desired protein to a nucleotide sequence encoding a polypeptide domain, which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAG extension/affinity purification system (Immunex Corp., Seattle, Wash.)

Diagnostics and Therapeutics

The data disclosed in this invention show that the nucleic acids and proteins of the invention and effector molecules thereof are useful in diagnostic and therapeutic applications implicated, for example but not limited to, in metabolic disorders such as obesity as well as related disorders such as metabolic syndrome, eating disorder, cachexia, diabetes mellitus, hypertension, coronary heart disease, hypercholesterolemia, dyslipidemia, osteoarthritis, and gallstones. Hence, diagnostic and therapeutic uses for the nucleic acids and proteins of the present invention, and homologous nucleic acids and proteins of the invention are, for example but not limited to, the following: (i) protein therapeutic, (ii) small molecule drug target, (iii) antibody target (therapeutic, diagnostic, drug targeting/cytotoxic antibody), (iv) diagnostic and/or prognostic marker, (v) gene therapy (gene delivery/gene ablation), (vi) research tools, and (vii) tissue regeneration in vitro and in vivo (regeneration for all these tissues and cell types composing these tissues and cell types derived from these tissues).

The nucleic acids and proteins of the invention-are useful in diagnostic and therapeutic applications implicated in various applications as described below. For example, but not limited to, cDNAs encoding the proteins of the invention and particularly their human homologues may be useful in gene therapy, and the proteins of the invention and particularly their human homologues may be useful when administered to a subject in need thereof. By way of non-limiting example, the compositions of the present invention will have efficacy for treatment of patients suffering from, for example, but not limited to, in metabolic disorders as described above.

The novel nucleic acid encoding a protein of the invention, or homologous proteins, or fragments thereof, may further be useful in diagnostic applications, wherein the presence or amount of the nucleic acids or the proteins are to be assessed. These materials are further useful in the generation of antibodies that bind immunospecifically to the novel substances of the invention for use in therapeutic or diagnostic methods.

For example, in one aspect, antibodies which are specific for a protein of the invention or homologous proteins may be used directly as an antagonist, or indirectly as a targeting or delivery mechanism for bringing a pharmaceutical agent to cells or tissue which express the protein. The antibodies may be generated using methods that are well known in the art. Such antibodies may include, but are not limited to, polyclonal, monoclonal, chimerical, single chain, Fab fragments, and fragments produced by a Fab expression library. Neutralising antibodies, (i.e., those which inhibit dimer formation) are especially preferred for therapeutic use.

For the production of antibodies, various hosts including goats, rabbits, rats, mice, humans, and others, may be immunized by injection with a protein of the invention or any fragment or oligopeptide thereof which has immunogenic properties. Depending on the host species, various adjuvants may be used to increase immunological response. It is preferred that the peptides, fragments, or oligopeptides used to induce antibodies have an amino acid sequence consisting of at least five amino acids, and more preferably at least 10 amino acids.

Monoclonal antibodies may be prepared using any technique that provides for the production of antibody molecules by continuous cell lines in culture. These include, but are not limited to, the hybridoma technique, the human B-cell hybridoma technique, and the EBV-hybridoma technique (Köhler, G. et al. (1975) Nature 256:495-497; Kozbor, D. et al. (1985) J. Immunol. Methods 81:31-42; Cote, R. J. et al. Proc. Natl. Acad. Sci. 80:2026-2030; Cole, S. P. et al. (1984) Mol. Cell Biol. 62:109-120).

In addition, techniques developed for the production of “chimeric antibodies”, the splicing of mouse antibody genes to human antibody genes to obtain a molecule with appropriate antigen specificity and biological activity can be used (Morrison, S. L. et al. (1984) Proc. Natl. Acad. Sci. 81:6851-6855; Neuberger, M. S. et al. (1984) Nature 312:604-608; Takeda, S. et al. (1985). Nature 314:452-454). Alternatively, techniques described for the production of single chain antibodies may be adapted, using methods known in the art, to produce specific single chain antibodies. Antibodies with related specificity, but of distinct idiotypic composition, may be generated by chain shuffling from random combinatorial immunoglobulin libraries (Burton, D. R. (1 991) Proc. Natl. Acad. Sci. 88:11120-3). Antibodies may also be produced by inducing in vivo production in the lymphocyte population or by screening recombinant immunoglobulin libraries or panels of highly specific binding reagents as disclosed in the literature (Orlandi, R. et al. (1989) Proc. Natl. Acad. Sci. 86:3833-3837; Winter, G. et al. (1991) Nature 349:293-299).

Antibody fragments, which contain specific binding sites for the proteins, may also be generated. For example, such fragments include, but are not limited to, the F(ab′)₂ fragments which can be produced by Pepsin digestion of the antibody molecule and the Fab fragments which can be generated by reducing the disulfide bridges of F(ab′)₂ fragments. Alternatively, Fab expression libraries may be constructed to allow rapid and easy identification of monoclonal Fab fragments with the desired specificity (Huse, W. D. et al. (1989) Science 254:1275-1281).

Various immunoassays may be used for screening to identify antibodies having the desired specificity. Numerous protocols for competitive binding and immunoradiometric assays using either polyclonal or monoclonal antibodies with established specificities are well known in the art. Such immunoassays typically involve the measurement of complex formation between a protein of the invention or homologous proteins and its specific antibody. A two-site, monoclonal-based immunoassay utilising monoclonal antibodies reactive to two non-interfering protein epitopes are preferred, but a competitive binding assay may also be employed (Maddox, supra).

In another embodiment of the invention, the polynucleotides or fragments thereof, or nucleic acid effector molecules such as antisense molecules, aptamers, RNAi molecules or ribozymes may be used for therapeutic purposes. In one aspect, aptamers, i.e. nucleic acid molecules, which are capable of binding to a protein of the invention and modulating its activity, may be generated by a screening and selection procedure involving the use of combinatorial nucleic acid libraries.

In a further aspect, antisense molecules to the polynucleotide may be used in situations in which it would be desirable to block the transcription of the mRNA. In particular, cells may be transformed with sequences complementary to polynucleotides encoding the proteins. Thus, antisense molecules may be used to modulate protein activity, or to achieve regulation of gene function. Such technology is now well know in the art, and sense or antisense oligomers or larger fragments, can be designed from various locations along the coding or control regions of sequences encoding the proteins. Expression vectors derived from retroviruses, adenoviruses, herpes or vaccinia viruses, or from various bacterial plasmids may be used for delivery of nucleotide sequences to the targeted organ, tissue or cell population. Methods, which are well known to those skilled in the art, can be used to construct recombinant vectors, which will express antisense molecules complementary to the polynucleotides of the genes encoding the proteins. These techniques are described both in Sambrook et al. (supra) and in Ausubel et al. (supra). Genes encoding a protein of the invention or homologous proteins can be turned off by transforming a cell or tissue with expression vectors which express high levels of polynucleotide or fragment thereof which encodes the protein. Such constructs may be used to introduce untranslatable sense or antisense sequences into a cell. Even in the absence of integration into the DNA, such vectors may continue to transcribe RNA molecules until they are disabled by endogenous nucleases. Transient expression may last for a month or more with a non-replicating vector and even longer if appropriate replication elements are part of the vector system.

As mentioned above, modifications of gene expression can be obtained by designing antisense molecules, e.g. DNA, RNA, or PNA, nucleic acid analogues such as to the control regions of the genes, i.e. the promoters, enhancers, and introns. Oligonucleotides derived from the transcription initiation site, e.g., between positions −10 and +10 from the start site, are preferred. Similarly, inhibition can be achieved using “triple helix” base-pairing methodology. Triple helix pairing is useful because it cause inhibition of the ability of the double helix to open sufficiently for the binding of polymerases, transcription factors, or regulatory molecules. Recent therapeutic advances using triplex DNA have been described in the literature (Gee, J. E. et al. (1994) In; Huber, B. E. and B. I. Carr, Molecular and Immunologic Approaches, Futura Publishing Co., Mt. Kisco, N.Y.). The antisense molecules may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. Examples, which may be used, include engineered hammerhead motif ribozyme molecules that can be specifically and efficiently catalyze endonucleolytic cleavage of sequences encoding the proteins. Specific ribozyme cleavage sites within any potential RNA target are initially identified by scanning the target molecule for ribozyme cleavage sites which include the following sequences: GUA, GUU, and GUC. Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridization with complementary oligonucleotides using ribonuclease protection assays.

Nucleic acid effector molecules, e.g. antisense molecules and ribozymes of the invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding the proteins. Such DNA sequences may be incorporated into a variety of vectors with suitable RNA polymerase promoters such as T7 or SP6. Alternatively, these cDNA constructs that synthesize antisense RNA constitutively or inducibly can be introduced into cell lines, cells, or tissues. RNA molecules may be modified to increase intracellular stability and half-life. Possible modifications include, but are not limited to, the addition of flanking sequences at the 5′ and/or 3′ ends of the molecule or the use of phosphorothioate or 2′ O-methyl rather than phosphodiesterase linkages within the backbone of the molecule. This concept is inherent in the production of PNAs and can be extended in all of these molecules by the inclusion of non-traditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection and by liposome injections may be achieved using methods, which are well known in the art. Any of the therapeutic methods described above may be applied to any suitable subject including, for example, mammals such as dogs, cats, cows, horses, rabbits, monkeys, and most preferably, humans.

An additional embodiment of the invention relates to the administration of a pharmaceutical composition, in conjunction with a pharmaceutically acceptable carrier, for any of the therapeutic effects discussed above. Such pharmaceutical compositions may comprise a protein of the invention is or homologous proteins, antibodies to a protein of the invention or homologous proteins, mimetics, agonists, antagonists, or inhibitors of a protein of the invention or homologous proteins. The compositions may be administered alone or in combination with at least one other agent, such as stabilizing compound, which may be administered in any sterile, biocompatible pharmaceutical carrier, including, but not limited to, saline, buffered saline, dextrose, and water. The compositions may be administered to a patient alone, or in combination with other agents, drugs or hormones. The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

In addition to the active ingredients, these pharmaceutical compositions may contain suitable pharmaceutically-acceptable carriers comprising excipients and auxiliaries, which facilitate processing of the active compounds into preparations which, can be used pharmaceutically. Further details on techniques for formulation and administration may be found in the latest edition of Remington's Pharmaceutical Sciences (Maack Publishing Co., Easton, Pa.).

The pharmaceutical compositions of the present invention may be manufactured in a manner that is known in the art, e.g., by means of conventional mixing, dissolving, granulating, dragee-making, levigating, emulsifying, encapsulating, entrapping, or lyophilizing processes. After pharmaceutical compositions have been prepared, they can be placed in an appropriate container and labeled for treatment of-an indicated condition. For administration of a protein of the invention or homologous proteins, such labeling would include amount, frequency, and method of administration.

Pharmaceutical compositions suitable for use in the invention include compositions wherein the active ingredients are contained in an effective amount to achieve the intended purpose. The determination of an effective dose is well within the capability of those skilled in the art. For any compounds, the therapeutically effective does can be estimated initially either in cell culture assays, e.g., of preadipocyte cell lines, or in animal models, usually mice, rabbits, dogs, or pigs. The animal model may also be used to determine the appropriate concentration range and route of administration. Such information can then be used to determine useful doses and routes for administration in humans. A therapeutically effective dose refers to that amount of active ingredient, for example a protein of the invention or homologous proteins, fragments thereof, antibodies of a protein of the invention or homologous proteins etc., which is effective for the treatment of a specific condition. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50. Pharmaceutical compositions, which exhibit large therapeutic indices, are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage from employed, sensitivity of the patient, and the route of administration. The exact dosage will be determined by the practitioner, in light of factors related to the subject that requires treatment. Dosage and administration are adjusted to provide sufficient levels of the active moiety or to maintain the desired effect.

Factors, which may be taken into account, include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation. Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art employ different formulations for nucleotides than for proteins or their inhibitors. Similarly, delivery of polynucleotides or polypeptides will be specific to particular cells, conditions, locations, etc.

In another embodiment, antibodies which specifically bind a protein of the invention or homologous proteins may be used for the diagnosis of conditions or diseases characterized by or associated with over- or underexpression of the proteins, or in assays to monitor patients being treated with the proteins, agonists, antagonists or inhibitors. The antibodies useful for diagnostic purposes may be prepared in the same manner as those described above for therapeutics. Diagnostic assays for the proteins include methods, which utilize the antibody and a label to detect a protein of the invention or homologous proteins in human body fluids or extracts of cells or tissues. The antibodies may be used with or without modification, and may be labeled by joining them, either covalently or non-covalently, with a reporter molecule. A wide variety of reporter molecules, which are known in the art may be used several of which are described above.

A variety of protocols including ELISA, RIA, and FACS for measuring a protein of the invention or homologous proteins are known in the art and provide a basis for diagnosing altered or abnormal levels of expression. Normal or standard values for protein expression are established by combining body fluids or cell extracts taken from normal mammalian subjects, preferably human, with antibodies to the proteins under conditions suitable for complex formation. The amount of standard complex formation may be quantified by various methods, but preferably by photometric means. Quantities of proteins expressed in control and disease samples, e.g. from biopsied tissues are compared with the standard values. Deviation between standard and subject values establishes the parameters for diagnosing disease.

In another embodiment of the invention, the polynucleotides specific for a protein of the invention or homologous nucleic acids may be used for diagnostic purposes. The polynucleotides, which may be used, include oligonucleotide sequences, antisense RNA and DNA molecules, and PNAs. The polynucleotides may be used to detect and quantitate gene expression in biopsied tissues in which expression of the proteins may be correlated with disease. The diagnostic assay may be used to distinguish between absence, presence, and excess expression of the genes, and to monitor regulation of expression levels during therapeutic intervention.

In one aspect, hybridization with probes, e.g. PCR probes which are capable of detecting polynucleotide sequences, including genomic sequences, encoding a protein of the invention or homologous proteins, or closely related molecules, may be used to identify nucleic acid sequences which encode the proteins. The specificity of the probe, whether it is made from a highly specific region, e.g. unique nucleotides in the 5′ regulatory region, or a less specific region, e.g. especially in the 3′ coding region, and the stringency of the hybridization or amplification (maximal, high, intermediate, or low) will determine whether the probe identifies only naturally occurring sequences encoding a protein of the invention or homologous proteins, alleles, or related sequences. Probes may also be used for the detection of related sequences, and should preferably contain at least 50% of the nucleotides from any of the protein-encoding sequences. The hybridization probes of the subject invention may be DNA or RNA and are preferably derived from the nucleotide sequence of the polynucleotide comprising a protein of the invention, or from a genomic sequence including promoter, enhancer elements, and introns of the naturally occurring sequences encoding a protein of the invention or homologous genes. Means for producing specific hybridization probes for DNAs encoding the proteins include the cloning of suitable nucleic acid sequences into vectors for the production of mRNA probes. Such vectors are known in the art, commercially available, and may be used to synthesize RNA probes in vitro by means of the addition of the appropriate RNA polymerases and the appropriate labeled nucleotides. Hybridization probes may be labeled by a variety of reporter groups, for example, radionuclides such as ³²p or ³⁵S, or enzymatic labels, such as alkaline phosphatase coupled to the probe via avidin/biotin coupling systems, and the like.

Polynucleotide sequences specific for a protein of the invention or homologous nucleic acids may be used for the diagnosis of conditions or diseases, which are associated with expression of the genes. Examples of such conditions or diseases include, but are not limited to, metabolic diseases and disorders, including obesity and diabetes. Polynucleotide sequences specific for the proteins of the invention or homologous nucleic acids may also be used to monitor the progress of patients receiving treatment for metabolic diseases and disorders, including obesity and diabetes. The polynucleotide sequences may be used in Southern or Northern analysis, dot blot, or other membrane-based technologies; in PCR technologies; or in dip stick, pin, ELISA or chip assays utilizing fluids or tissues from patient biopsies to detect altered gene expression. Such qualitative or quantitative methods are well known in the art.

In a particular aspect, the nucleotide sequences specific for a protein of the invention or homologous nucleic acids may be useful in assays that detect activation or induction of various metabolic diseases such as obesity as is well as related disorders such as metabolic syndrome, eating disorder, cachexia, diabetes mellitus, hypertension, coronary heart disease, hypercholesterolemia, dyslipidemia, osteoarthritis, and gallstones. The nucleotide sequences may be labeled by standard methods, and added to a fluid or tissue sample from a patient under conditions suitable for the formation of hybridization complexes. After a suitable incubation period, the sample is washed and the signal is quantitated and compared with a standard value. The presence of altered levels of nucleotide sequences in the sample indicates the presence of the associated disease. Such assays may also be used to evaluate the efficacy of a particular therapeutic treatment regimen in animal studies, in clinical trials, or in monitoring the treatment of an individual patient.

In order to provide a basis for the diagnosis of a disease associated with expression of a protein of the invention or homologous proteins, a normal or standard profile for expression is established. This may be accomplished by combining body fluids or cell extracts taken from normal subjects, either animal or human, with a sequence, or a fragment thereof, which is specific for a protein of the invention or homologous nucleic acids, under conditions suitable for hybridization or amplification. Standard hybridization may be quantified by comparing the values obtained from normal subjects with those from an experiment where a known amount of a substantially purified polynucleotide is used. Standard values obtained from normal samples may be compared with values obtained from samples from patients who are symptomatic for disease. Deviation between standard and subject values is used to establish the presence of disease. Once disease is established and a treatment protocol is initiated, hybridization assays may be repeated on a regular basis to evaluate whether the level of expression in the patient begins to approximate that, which is observed in the normal patient. The results obtained from successive assays may be used to show the efficacy of treatment over a period ranging from several days to months.

With respect to metabolic diseases such as obesity as well as related disorders such as metabolic syndrome, eating disorder, cachexia, diabetes mellitus, hypertension, coronary heart disease, hypercholesterolemia, dyslipidemia, osteoarthritis, and gallstones, the presence of a relatively high amount of transcript in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the metabolic diseases and disorders. Additional diagnostic uses for oligonucleotides designed from the sequences encoding a protein of the invention or homologous proteins may involve the use of PCR. Such oligomers may be chemically synthesized, generated enzymatically, or produced from a recombinant source. Oligomers will preferably consist of two nucleotide sequences, one with sense orientation (5′.fwdarw.3′) and another with antisense (3′.rarw.5′), employed under optimized conditions for identification of a specific gene or condition. The same two oligomers, nested sets of oligomers, or even a degenerate pool of oligomers may be employed under less stringent conditions for detection and/or quantification of closely related DNA or RNA sequences.

Methods which may also be used to quantitate the expression of a protein of the invention include radiolabeling or biotinylating nucleotides, coamplification of a control nucleic acid, and standard curves onto which the experimental results are interpolated (Melby, P. C. et al. (1 993) J. Immunol. Methods, 159:235-244; Duplaa, C. et al. (1993) Anal. Biochem. 21 2:229-236). The speed of quantification of multiple samples may be accelerated by running the assay in an ELISA format where the oligomer of interest is presented in various dilutions and a spectrophotometric or calorimetric response gives rapid quantification.

In another embodiment of the invention, the nucleic acid sequences, which encode a protein of the invention or homologous proteins, may also be used to generate hybridization probes, which are useful for mapping the naturally occurring genomic sequence. The sequences may be mapped to a particular chromosome or to a specific region of the chromosome using well known techniques. Such techniques include FISH, FACS, or artificial chromosome constructions, such as yeast artificial chromosomes, bacterial artificial chromosomes, bacterial P1 constructions or single chromosome cDNA libraries as reviewed in Price, C. M. (1993) Blood Rev. 7:127-134, and Trask, B. J. (1991) Trends Genet. 7:149-154. FISH (as described in Verma et al. (1 988) Human Chromosomes: A Manual of Basic Techniques, Pergamon Press, New York, N.Y.) may be correlated with other physical chromosome mapping techniques and genetic map data. Examples of genetic map data can be found in the 1994 Genome Issue of Science (265:1981f). Correlation between the location of the gene encoding a protein of the invention on a physical chromosomal map and a specific disease, or predisposition to a specific disease, may help to delimit the region of DNA associated with that genetic disease.

The nucleotide sequences of the subject invention may be used to detect differences in gene sequences between normal, carrier, or affected individuals. An analysis of polymorphisms, e.g. single nucleotide polymorphisms may be carried out. Further, in situ hybridization of chromosomal preparations and physical mapping techniques such as linkage analysis using established chromosomal markers may be used for extending genetic maps. Often the placement of a gene on the chromosome of another mammalian species, such as mouse, may reveal associated markers even if the number or arm of a particular human chromosome is not known. New sequences can be assigned to chromosomal arms, or parts thereof, by physical mapping. This provides valuable information to investigators searching for disease genes using positional cloning or other gene discovery techniques. Once the disease or syndrome has been crudely localized by genetic linkage to a particular genomic region, for example, AT to 11q22-23 (Gatti, R. A. et al. (1988) Nature 336:577-580), any sequences mapping to that area may represent associated or regulatory genes for further investigation. The nucleotide sequences of the subject invention may also be used to detect differences in the chromosomal location due to translocation, inversion, etc. among normal, carrier, or affected individuals.

In another embodiment of the invention, a protein of the invention or homologous proteins, their catalytic or immunogenic fragments or oligopeptides thereof, can be used for screening libraries of compounds, e.g. peptides or low-molecular weight organic compounds, in any of a variety of drug screening techniques.

One can identify effectors, e.g. receptors, enzymes, proteins, ligands, or substrates that bind to, modulate or mimic the action of one or more of the proteins of the invention. The protein or fragment employed in such screening may be free in solution, affixed to a solid support, borne on a cell surface, or located intracellularly. The formation of binding complexes, between a protein of the invention and the agent tested, may be measured.

Agents can also be identified, which either directly or indirectly, influence the activity of the proteins of the invention.

The cAMP-dependent protein kinase (PKA) holoenzyme consists of 2 regulatory (PRKAR2A and PRKAR2B are two of several regulatory subunits) and 2 catalytic subunits that dissociate upon the binding of 2 cAMP molecules to each of the regulatory subunits. The free, activated catalytic subunits then catalyze the phosphorylation of specific substrate proteins. In vivo, the enzymatic kinase activity of the unmodified polypeptides of PKA, or homologues thereof, towards a substrate can be measured. Activation of the kinase may be induced in the natural context by extracellular or intracellular stimuli, such as signaling molecules or environmental influences. One may generate a system containing the regulatory and catalytic subunits of PKA, or homologues thereof, may it be an organism, a tissue, a culture of cells or cell-free environment, by exogenously applying this stimulus or by mimicking this stimulus by a variety of the techniques, some of them described further below. A system containing the regulatory and catalytic subunits of PKA, or homologues thereof may be produced (i) for the purpose of diagnosis, study, prevention, and treatment of diseases and disorders related to body-weight regulation and thermogenesis, for example, but not limited to, metabolic diseases, (ii) for the purpose of identifying or validating therapeutic candidate agents, pharmaceuticals or drugs that influence the genes of the invention or their encoded polypeptides, (iii) for the purpose of generating cell lysates containing activated polypeptides encoded by the genes of the invention, (iv) for the purpose of isolating from this source activated polypeptides encoded by the genes of the invention.

In one embodiment of the invention, one may produce activated PKA independent of the natural stimuli for the above said purposes by, for example, but not limited to, (i) an agent that mimics the natural stimulus; (ii) an agent, that acts downstream of the natural stimulus, such as activators of PKA, constitutive active alleles of PKA itself as they are described or may be developed; (iii) by introduction of single or multiple amino acid substitutions, deletions or insertions within the sequence of PKA to yield constitutive active forms; (iv) by the use of isolated fragments of PKA. In addition, one may generate enzymatically active PKA in an ectopic system, prokaryotic or eukaryotic, in vivo or in vitro, by co-transfering to this system the activating components.

In addition, activity of SRM, NPFF2, NPFF1, OX1R, OX2R, PRKAR2A, PRKAR2B, or FtsJ2 homolog proteins against their physiological substrate(s) or derivatives thereof can be measured in cell-based assays. In case of NPFF2, NPFF1, OX1R, or OX2R, agents could also interfere with or mimic the binding of ligands to its receptor, thereby showing antagonists' of agonists' properties. In case of SLC25A11, agents may regulate the oxoglutarate/malate carrier activity of the decoupling activity of SLC25A11. Agents may also interfere with posttranslational modifications of the proteins of the invention, such as phosphorylation and dephosphorylation, farnesylation, palmitoylation, acetylation, alkylation, ubiquitination, proteolytic processing, subcellular localization, and degradation. Moreover, agents can influence the dimerization or oligomerization of the proteins of the invention, particularly of SLC25A11, Cab45, AWP1, or ZNF216 or, in a heterologous manner, of the proteins of the invention with other proteins, for example, but not exclusively, docking proteins, enzymes, receptors, ion channels, uncoupling proteins or translation factors. Agents can also act on the physical interaction of the proteins of this invention with other proteins, which are required for protein function, for example, but not exclusively, their downstream signaling.

Methods for determining protein-protein interaction are well known in the art. For example, binding of a fluorescently labeled peptide derived from a protein of the invention to the interacting protein (or vice versa), can be detected by a change in polarisation. In case that both binding partners, which can be either the full length proteins as well fragments thereof are fluorescently labeled, binding can be detected by fluorescence energy transfer (FRET) from one fluorophore to the other. In addition, a variety of commercially available assay principles suitable for detection of protein-protein interaction are well known in the art, for example but not exclusively AlphaScreen (PerkinElmer) or Scintillation Proximity Assays (SPA) by Amersham. Alternatively, the interaction of the proteins of the invention with cellular proteins can be the basis for a cell-based screening assay, in which both proteins are fluorescently labeled and interaction of both proteins is detected by analyzing cotranslocation of both proteins with a cellular imaging reader, as developed for example, but not exclusively, by Cellomics or EvotecOAI. In all cases, the two or more binding partners can be different proteins with one being a protein of the invention, or in case of dimerization and/or oligomerization the protein of the invention itself. Proteins of the invention, for which one target mechanism of interest, but not the only one, would be such protein/protein interactions are SLC25A11, Cab45, AWP1, or ZNF216.

Assays for determining enzymatic and carrier activity of the proteins of the invention are well known in the art. Well known in the art are also a variety of assay formats to measure receptor/ligand binding.

Of particular interest are screening assays for agents that have a low toxicity for mammalian cells. The term “agent” as used herein describes any molecule, e.g. protein or pharmaceutical, with the capability of altering or mimicking the physiological function of one or more of the proteins of the invention. Candidate agents encompass numerous chemical classes, though typically they are organic molecules, preferably small organic compounds having a molecular weight of more than 50 and less than about 2,500 Daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise carbocyclic or heterocyclic structures and/or aromatic or polyaromatic structures substituted with one or more of the above functional groups.

Candidate agents are also found among biomolecules including peptides, saccharides, fatty acids, steroids, purines, pyrimidines, nucleic acids and derivatives, structural analogs or combinations thereof. Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Where the screening assay is a binding assay, one or more of the molecules may be joined to a label, where the label can directly or indirectly provide a detectable signal.

Candidate agents may also be found in kinase assays where a kinase substrate such as a protein, a peptide, a lipid, or an organic compound, which may or may not include modifications as further described below, or others are phosphorylated by the proteins or protein fragments of the invention. A therapeutic candidate agent may be identified by its ability to increase or decrease the enzymatic activity of the proteins of the invention. The kinase activity may be detected by change of the chemical, physical or immunological properties of the substrate due to phosphorylation. One example could be the transfer of radioisotopically labelled phosphate groups from an appropriate donor molecule to the kinase substrate catalyzed by the polypeptides of the invention. The phosphorylation of the substrate may be followed by detection of the substrates autoradiography with techniques well known in the art.

Yet in another example, the change of mass of the substrate due to its phosphorylation may be detected by mass spectrometry techniques. One could also detect the phosphorylation status of a substrate with an analyte discriminating between the phosphorylated and unphosphorylated status of the substrate. Such an analyte may act by having different affinities for the phosphorylated and unphosphorylated forms of the substrate or by having specific affinity for phosphate groups. Such an analyte could be, but is not limited to, an antibody or antibody derivative, a recombinant antibody-like structure, a protein, a nucleic acid, a molecule containing a complexed metal ion, an anion exchange chromatography matrix, an affinity chromatography matrix or any other molecule with phosphorylation dependend selectivity towards the substrate.

Such an analyte could be employed to detect the kinase substrate, which is immobilized on a solid support during or after an enzymatic reaction. If the analyte is an antibody, its binding to the substrate could be detected by a variety of techniques as they are described in Harlow and Lane, 1998, Antibodies, CSH Lab Press, NY. If the analyte molecule is not an antibody, it may be detected by virtue of its chemical, physical or immunological properties, being endogenously associated with it or engineered to it.

Yet in another example the kinase substrate may have features, designed or endogenous, to facilitate its binding or detection in order to generate a signal that is suitable for the analysis of the substrates phosphorylation status. These features may be, but are not limited to, a biotin molecule or derivative thereof, a glutathione-S-transferase moiety, a moiety of six or more consecutive histidine residues, an amino acid sequence or hapten to function as an epitope tag, a fluorochrome, an enzyme or enzyme fragment. The kinase substrate may be linked to these or other features with a molecular spacer arm to avoid steric hindrance.

In one example, the kinase substrate may be labelled with a fluorochrome. The binding of the analyte to the labelled substrate in solution may be followed by the technique of fluorescence polarization as it is described in the literature (see, for example, Deshpande, S. et al. (1999) Prog. Biomed. Optics (SPIE) 3603:261; Parker, G. J. et al. (2000) J. Biomol. Screen. 5:77-88; Wu, P. et al. (1997) Anal. Biochem. 249:29-36). In a variation of this example, a fluorescent tracer molecule may compete with the substrate for the analyte to detect kinase activity by a technique which is known to those skilled in the art as indirect fluorescence polarization.

Another technique for drug screening, which may be used, provides for high throughput screening of compounds having suitable binding affinity to the protein of interest as described in published PCT application WO84/03564. In this method, as applied to a protein of the invention or homologous proteins large numbers of different small test compounds are synthesized on a solid substrate, such as plastic pins or some other surface. The test compounds are reacted with the protein, or a fragment thereof, and washed. Bound protein is then detected by methods well known in the art. Purified protein can also be coated directly onto plates for use in the aforementioned drug screening techniques. Alternatively, non-neutralizing antibodies can be used to capture the peptide and immobilize it on a solid support. In another embodiment, one may use competitive drug screening assays in which neutralizing antibodies against the protein specifically compete with a test compound for binding the protein. In this manner, the antibodies can be used to detect the presence of any peptide, which shares one or more antigenic determinants with a protein of the invention or homologous proteins.

In another embodiment, one may use competitive drug screening assays in which neutralising antibodies capable of binding a protein of the invention specifically compete with a test compound for binding said protein of the invention. In this manner, the antibodies can be used to detect the presence of any peptide, which shares one or more antigenic determinants with said protein of the invention.

The nucleic acids encoding the proteins of the invention can be used to generate transgenic cell lines and animals. These transgenic non-human animals are useful in the study of the function and regulation of the proteins of the invention in vivo. Transgenic animals, particularly mammalian transgenic animals, can serve as a model system for the investigation of many developmental and cellular processes common to humans. A variety of non-human models of metabolic disorders can be used to test modulators of the protein of the invention. Misexpression (for example, overexpression or lack of expression) of the protein of the invention, particular feeding conditions, and/or administration of biologically active compounts can create models of metablic disorders.

In one embodiment of the invention, such assays use mouse models of insulin resistance and/or diabetes, such as mice carrying gene knockouts in the leptin pathway (for example, ob (leptin) or db (leptin receptor) mice). Such mice develop typical symptoms of diabetes, show hepatic lipid accumulation and frequently have increased plasma lipid levels (see Bruning et al, 1998, Mol. Cell. 2:449-569). Susceptible wild type mice (for example C57BI/6) show similiar symptoms if fed a high fat diet. In addition to testing the expression of the proteins of the invention in such mouse strains (see EXAMPLES section), these mice could be used to test whether administration of a candidate modulator alters for example lipid accumulation in the liver, in plasma, or adipose tissues using standard assays well known in the art, such as FPLC, calorimetric assays, blood glucose level tests, insulin tolerance tests and others.

Transgenic animals may be made through homologous recombination in embryonic stem cells, where the normal locus of the gene encoding the protein of the invention is mutated. Alternatively, a nucleic acid construct encoding the protein is injected into oocytes and is randomly integrated into the genome. One may also express the genes of the invention or variants thereof in tissues where they are not normally expressed or at abnormal times of development. Furthermore, variants of the genes of the invention like specific constructs expressing anti-sense molecules or expression of dominant negative mutations, which will block or alter the expression of the proteins of the invention may be randomly integrated into the genome. A detectable marker, such as lac Z or luciferase may be introduced into the locus of the genes of the invention, where upregulation of expression of the genes of the invention will result in an easily detectable change in phenotype. Vectors for stable integration include plasmids, retroviruses and other animal viruses, yeast artificial chromosomes (YACs), and the like. DNA constructs for homologous recombination will contain at least portions of the genes of the invention with the desired genetic modification, and will include regions of homology to the target locus. Conveniently, markers for positive and negative selection are included. DNA constructs for random integration do not need to contain regions of homology to mediate recombination. DNA constructs for random integration will consist of the nucleic acids encoding the proteins of the invention, a regulatory element (promoter), an intron and a poly-adenylation signal. Methods for generating cells having targeted gene modifications through homologous recombination are known in the field.

For embryonic stem (ES) cells, an ES cell line may be employed, or embryonic cells may be obtained freshly from a host, e.g. mouse, rat, guinea pig, etc. Such cells are grown on an appropriate fibroblast-feeder layer and are grown in the presence of leukemia inhibiting factor (LIF). ES or embryonic cells may be transfected and can then be used to produce transgenic animals. After transfection, the ES cells are plated onto a feeder layer in an appropriate medium. Cells containing the construct may be selected by employing a selection medium. After sufficient time for colonies to grow, they are picked and analyzed for the occurrence of homologous recombination. Colonies that are positive may then be used for embryo manipulation and morula aggregation. Briefly, morulae are obtained from 4 to 6 week old superovulated females, the Zona Pellucida is removed and the morulae are put into small depressions of a tissue culture dish. The ES cells are trypsinized, and the modified cells are placed into the depression closely to the morulae. On the following day the aggregates are transfered into the uterine horns of pseudopregnant females. Females are then allowed to go to term. Chimeric offsprings can be readily detected by a change in coat color and are subsequently screened for the transmission of the mutation into the next generation (F1-generation). Offspring of the F1-generation are screened for the presence of the modified gene and males and females having the modification are mated to produce homozygous progeny. If the gene alterations cause lethality at some point in development, tissues or organs can be maintained as allogenic or congenic grafts or transplants, or in vitro culture. The transgenic animals may be any non-human mammal, such as laboratory animal, domestic animals, etc., for example, mouse, rat, guinea pig, sheep, cow, pig, and others. The transgenic animals may be used in functional studies, drug screening, and other applications and are useful in the study of the function and regulation of the proteins of the invention in vivo.

Finally, the invention also relates to a kit comprising at least one of
(a) a SRM, NPFF2, NPFF1, OX1R, OX2R, SLC25A11, PRKAR2A, PRKAR2B, Cab45, FtsJ2, AWP1, or ZNF216 nucleic acid molecule or a fragment thereof;
(b) a SRM, NPFF2, NPFF1, OX1R, OX2R, SLC25A11, PRKAR2A, PRKAR2B, Cab45, FtsJ2, AWP1, or ZNF216 amino acid molecule or a fragment or an isoform thereof;
(c) a vector comprising the nucleic acid of (a);
(d) a host cell comprising the nucleic acid of (a) or the vector of (b);
(e) a polypeptide encoded by the nucleic acid of (a);
(f) a fusion polypeptide encoded by the nucleic acid of (a);
(g) an effector, e.g. an antibody, an aptamer or another receptor against the nucleic acid of (a) or the polypeptide of (b), (e) or (f) and
(g) an anti-sense oligonucleotide of the nucleic acid of (a).

The kit may be used for diagnostic or therapeutic purposes or for screening applications as described above. The kit may further contain user instructions.

The Figures show:

FIG. 1 shows the triglyceride content of a spermidine synthase (GadFly Accession Number CG8327) mutant. Shown is the increase of triglyceride content of EP(3)3054 flies (referred to as ‘EP(3)3054’) caused by homozygous viable integration of the P-vector in comparison to controls with integration of this vector type elsewhere in the genome (referred to as ‘EP-control’).

FIG. 2 shows the molecular organisation of the mutated spermidine synthase (Gadfly Accession Number CG8327) gene locus.

FIG. 3 shows the mammalian homologs of GadFly Accession Number CG8327.

FIG. 3A shows the BLASTP search result for the CG8327 gene product (Query) with the best human homolog match (Sbject).

FIG. 3B shows the nucleic acid sequence of human spermidine synthase (SEQ ID NO: 1; GenBank Accession Number NM_{—}003132).

FIG. 3C shows the amino acid sequence (one-letter code) of human spermidine synthase (SEQ ID NO: 2; GenBank Accession Number NP_{—}003123).

FIG. 3D shows the Clustal X (1.81) protein sequence alignment for human spermidine synthase (referred to as ‘Hs NP_{—}003123’), murine spermidine synthase (referred to as ‘Mm NP_{—}033298’), and Drosophila spermidine synthase (referred to as ‘Dm CG8327’). Gaps in the alignment are represented as -.

FIG. 4 shows the expression of spermidine synthase in mammalian tissues. The relative RNA-expression is shown on the Y-axis. In FIG. 4A and B, the tissues tested are given on the X-axis. “WAT” refers to white adipose tissue, “BAT” refers to brown adipose tissue. In FIG. 4C and D, the X-axis represents the time axis. “d0” refers to day 0 (start of the experiment), “d2”-“d10” refers to day 2-day 10 of adipocyte differentiation.

FIG. 4A: Real-time PCR analysis of spermidine synthase expression in wildtype mouse tissues.

FIG. 4B: Real-time PCR mediated analysis of spermidine synthase expression in different mouse models.

FIG. 4C: Real-time PCR mediated analysis of spermidine synthase expression during the differentiation of 3T3-L1 cells from preadipocytes to mature adipocytes.

FIG. 4D: Real-time PCR mediated analysis of spermidine synthase expression during the differentiation of 3T3-F442A cells from preadipocytes to mature adipocytes.

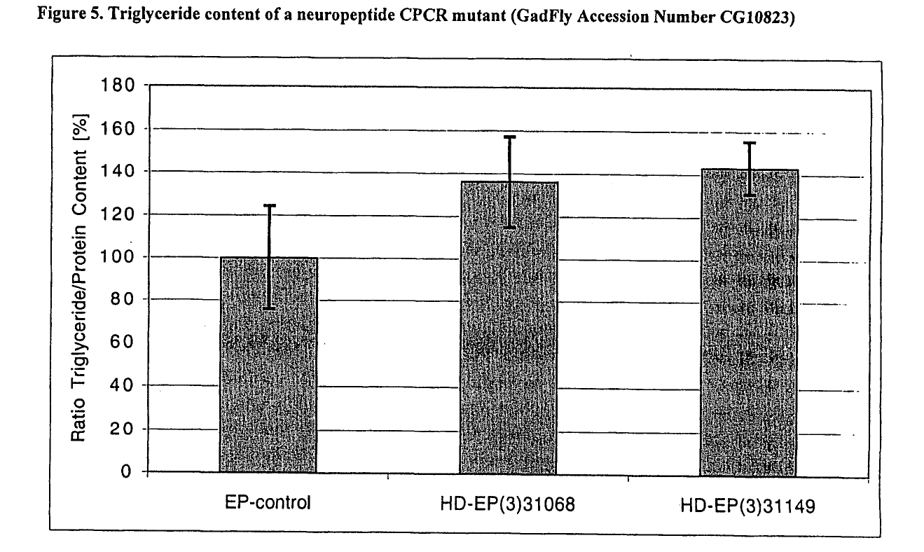

FIG. 5 shows the triglyceride content of two GPCR (GadFly Accession Number CG10823) mutants. Shown is the increase of triglyceride content of HD-EP(3)31068 flies (referred to as ‘HD-EP(3)31068’; column 2) and HD-EP(3)31149 flies (referred to as ‘HD-EP(3)31149’; column 3) caused by homozygous viable integration of the P-vector in comparison to controls with integration of this vector elsewhere in the genome (referred to as ‘EP-control’; column 1).

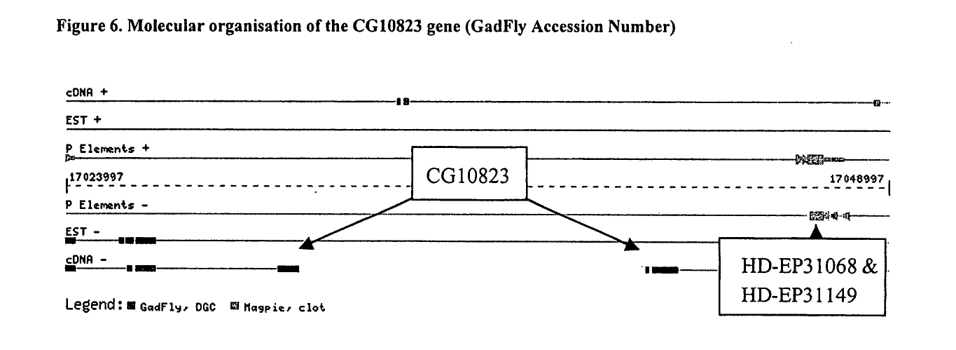

FIG. 6 shows the molecular organization of the mutated gene locus of Gadfly Accession Number CG10823.

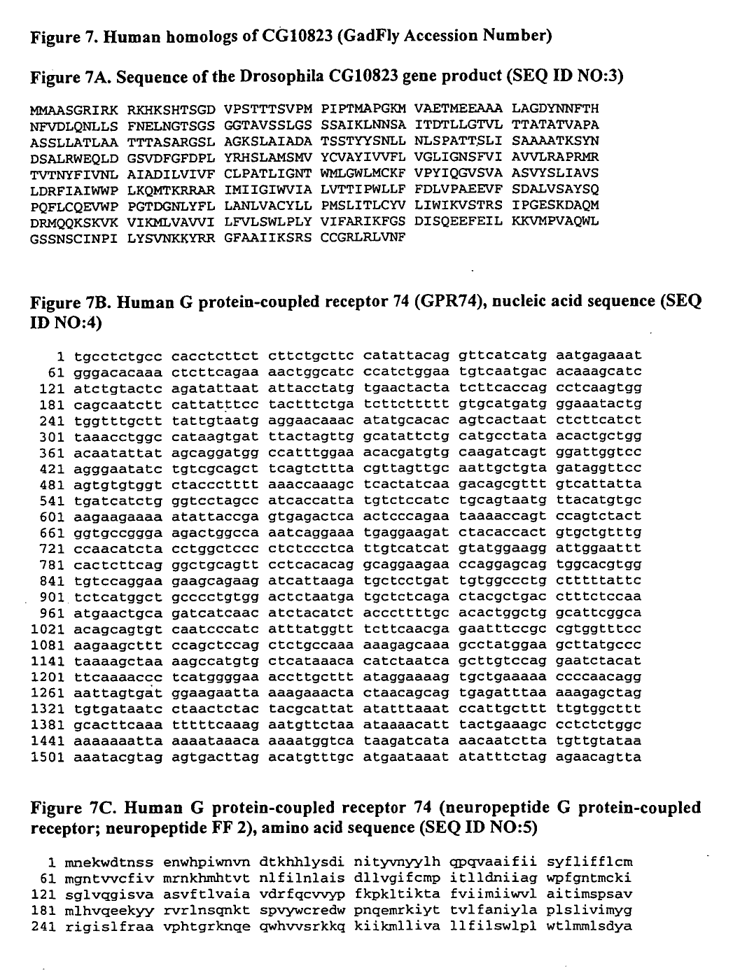

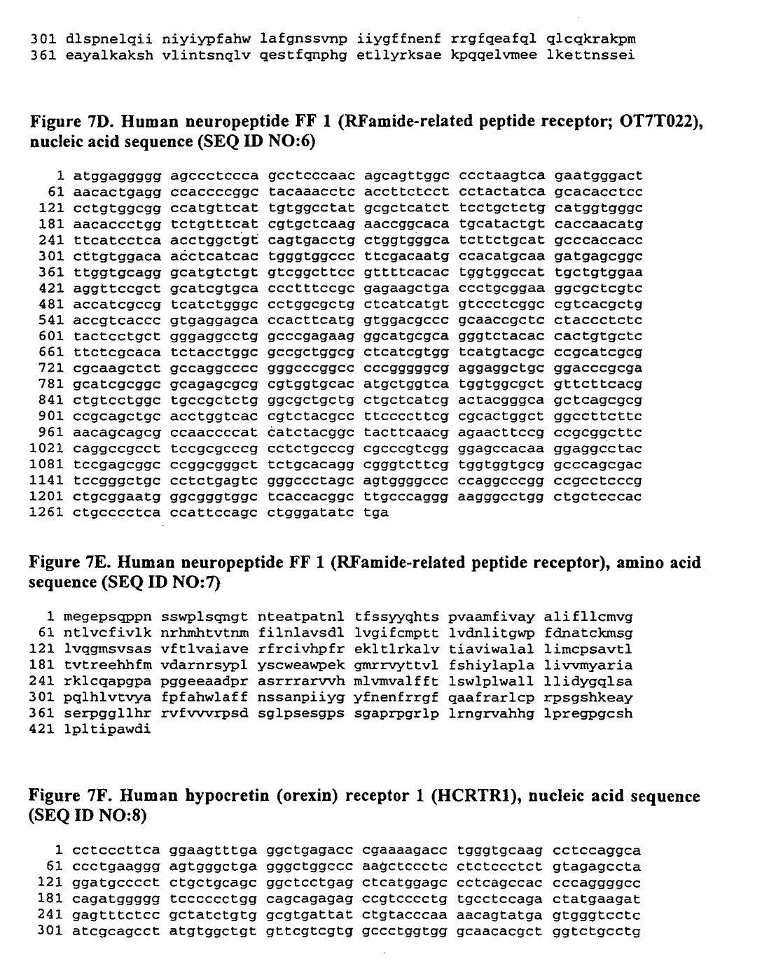

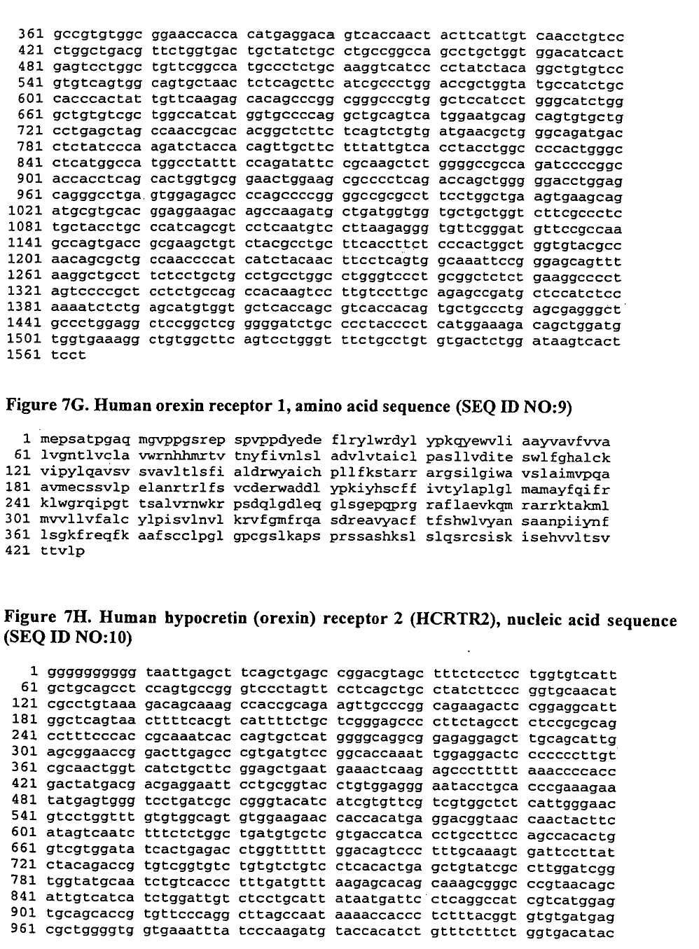

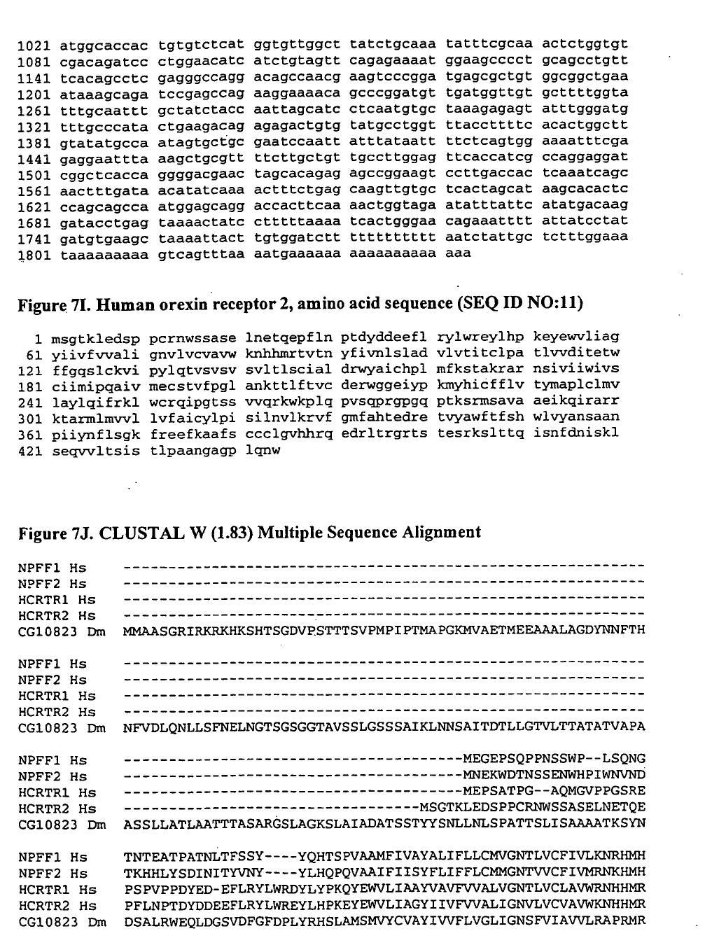

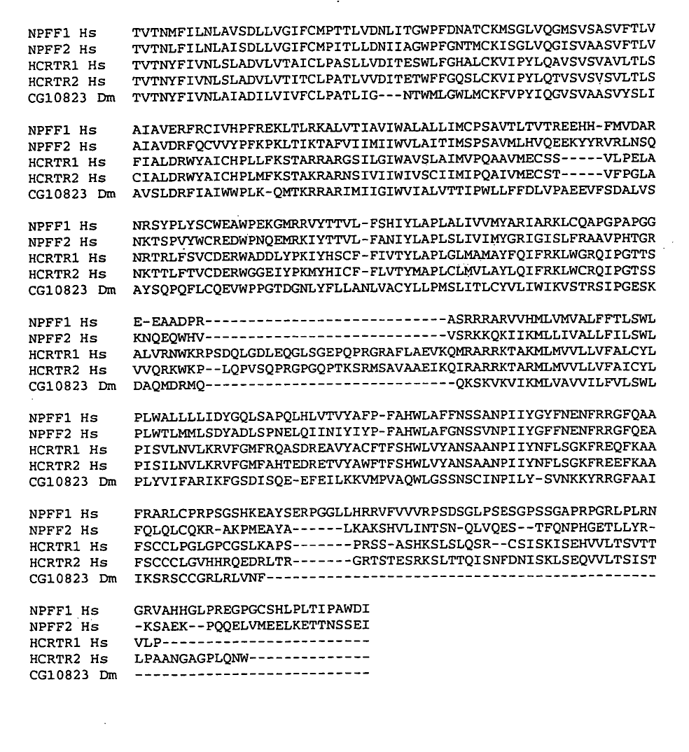

FIG. 7 shows the amino acid sequence of GadFly Accession NumberCG10823 and the human homologs of CG10823.

FIG. 7A shows the amino acid sequence of Drosophila CG10823 protein (SEQ ID NO: 3).

FIG. 7B shows the nucleic acid sequence of human G protein-coupled receptor 74 (also referred to as neuropeptide FF 2; SEQ ID NO: 4; GenBank Accession Number NM_{—}053036).

FIG. 7C shows the amino acid sequence (one-letter code) of human neuropeptide FF 2 (SEQ ID NO: 5; GenBank Accession Number NP_{—}444264).

FIG. 7D shows the nucleic acid sequence of human neuropeptide FF 1 (SEQ ID NO: 6; GenBank Accession Number NM_{—}022146).

FIG. 7E shows the amino acid sequence (one-letter code) of human neuropeptide FF 1 (SEQ ID NO: 7; GenBank Accession Number NP_{—}071429).

FIG. 7F shows the nucleic acid sequence of human orexin receptor 1 (SEQ ID NO: 8; GenBank Accession Number NM_{—}001525).

FIG. 7G shows the amino acid sequence (one-letter code) of human orexin receptor 1 (SEQ ID NO: 9; GenBank Accession Number NP_{—}001516).

FIG. 7H shows the nucleic acid sequence of human orexin receptor 2 (SEQ ID NO:10; GenBank Accession Number NM_{—}001526).

FIG. 7I shows the amino acid sequence (one-letter code) of human orexin receptor 2 (SEQ ID NO: 11; GenBank Accession Number NP_{—}001517).

FIG. 7J shows the Claustal W (1.83) multiple sequence alignment analysis of human neuropeptide FF1 receptor (referred to as ‘NPFF1 Hs’), human neuropeptide FF2 receptor (referred to as ‘NPFF2 Hs’), human orexin receptor 1 (referred to as ‘HCRTR1 Hs’), human orexin receptor 2 (referred to as ‘HCRTR2 Hs’), and Drosophila CG10823 protein (referred to as ‘CG10823 Dm’). Gaps in the alignment are represented as -.

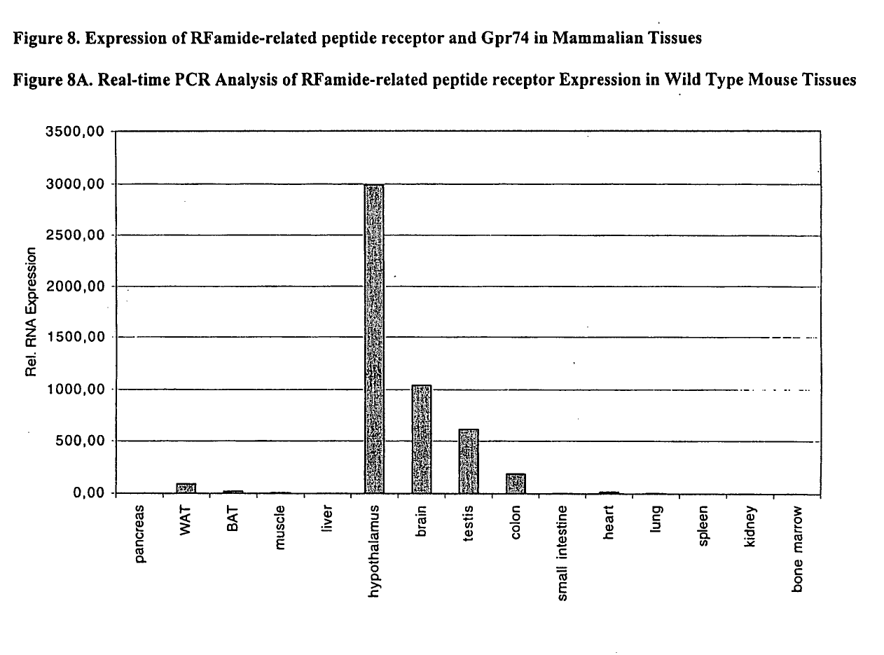

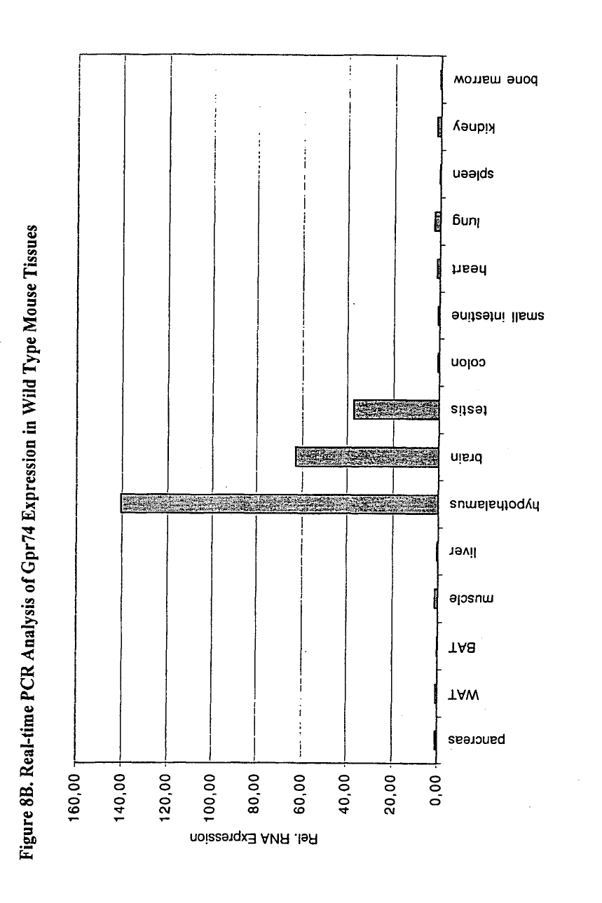

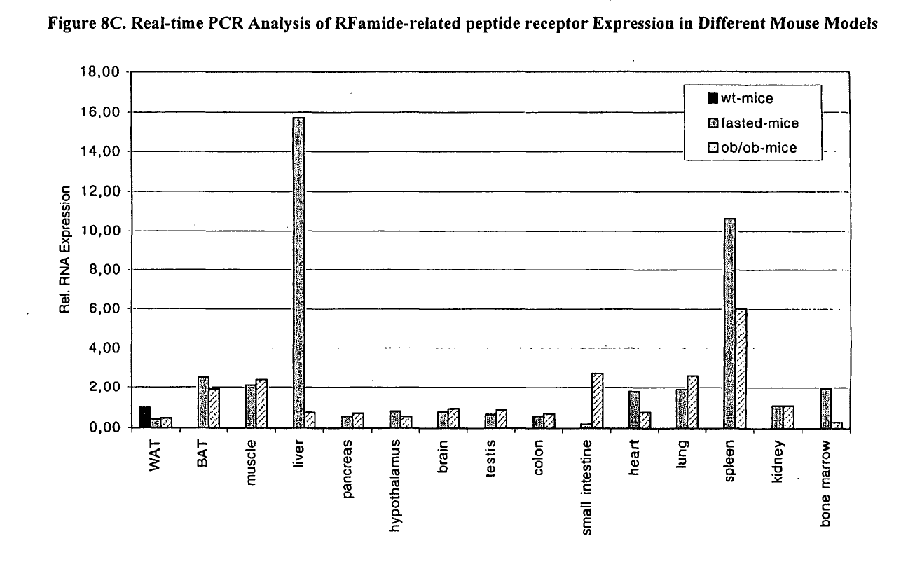

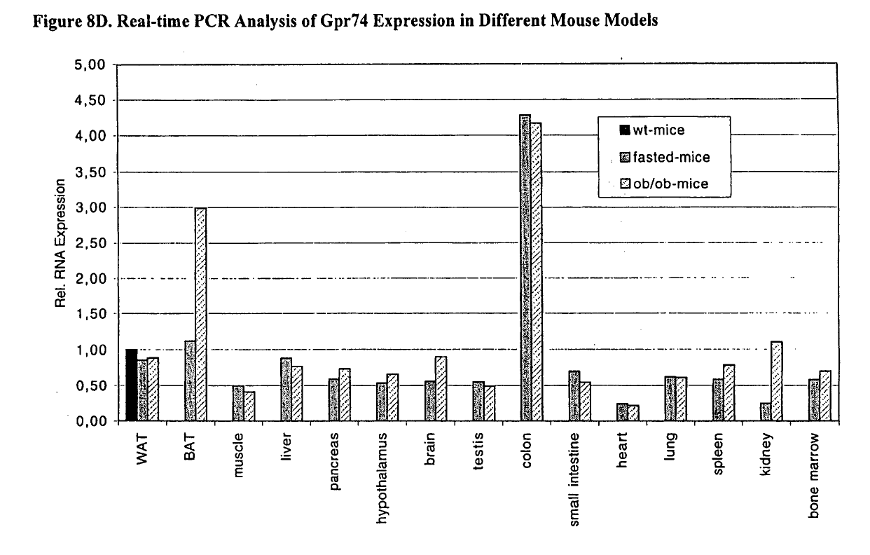

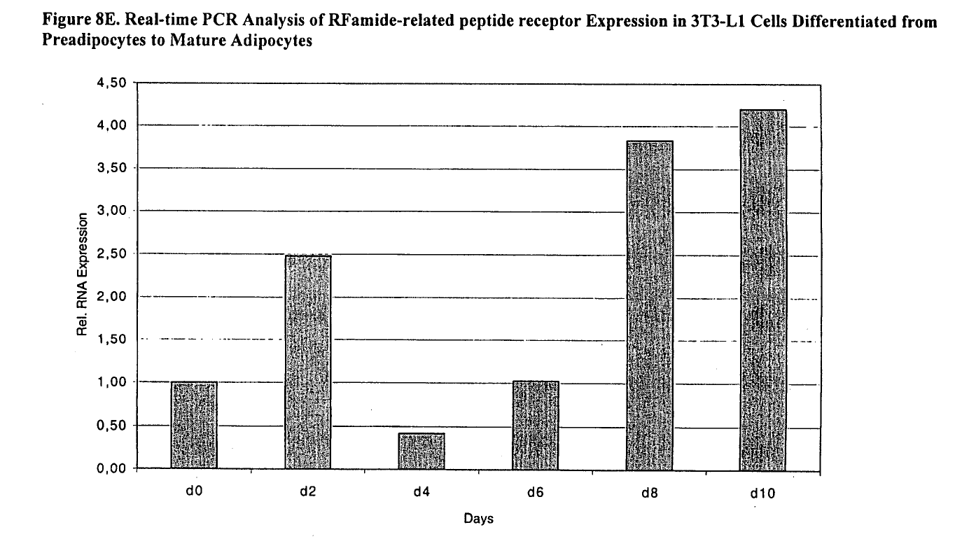

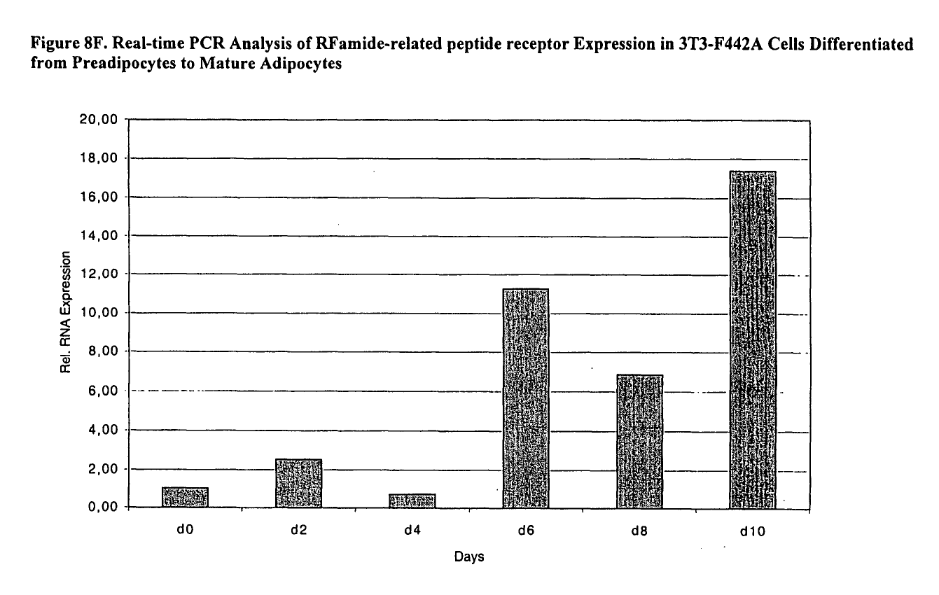

FIG. 8 shows the expression of Gpr74 (NPFF2 homolog) and RFamide-related peptide receptor (NPFF1 homolog) in mammalian tissues. The relative RNA-expression is shown on the Y-axis. In FIG. 8A, B, C, and D, the tissues tested are given on the X-axis. “WAT” refers to white adipose tissue, “BAT” refers to brown adipose tissue. In FIG. 8E and F, the X-axis represents the time axis. “d0” refers to day 0 (start of the experiment), “d2”-“d10” refers to day 2-day 10 of adipocyte differentiation.

FIG. 8A: Real-time PCR analysis of RFamide-related peptide receptor expression in wildtype mouse tissues.

FIG. 8B: Real-time PCR analysis of Gpr74 expression in wildtype mouse tissues.

FIG. 8C: Real-time PCR mediated analysis of RFamide-related peptide receptor expression in different mouse models.

FIG. 8D: Real-time PCR mediated analysis of Gpr74 expression in different mouse models.

FIG. 8E: Real-time PCR mediated analysis of RFamide-related peptide receptor expression during the differentiation of 3T3-L1 cells from preadipocytes to mature adipocytes.

FIG. 8F: Real-time PCR mediated analysis of RFamide-related peptide receptor expression during the differentiation of 3T3-F442A cells from preadipocytes to mature adipocytes.

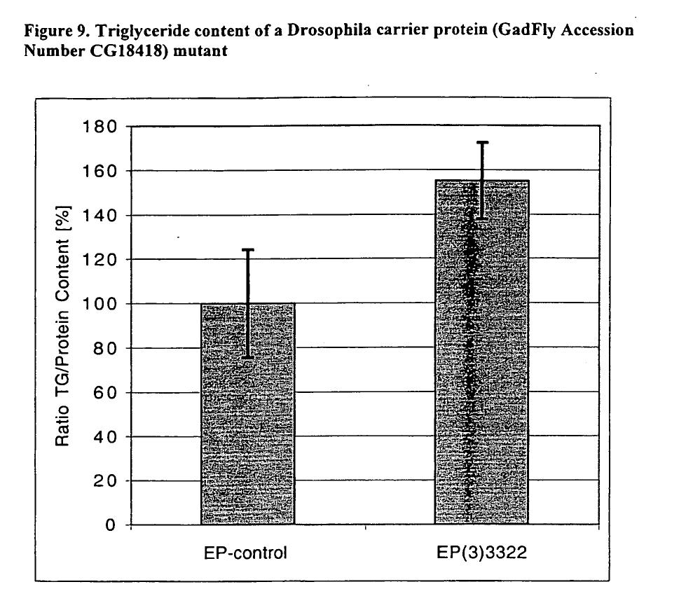

FIG. 9 shows the triglyceride content of a Drosophila carrier protein (Gadfly Accession Number CG18418) mutant. Shown is the increase of triglyceride content of EP(3)3322 flies (referred to as ‘EP(3)3322’) caused by homozygous viable integration of the P-vector in comparison to controls with integration of these vectors elsewhere in the genome (referred to as ‘EP control’).

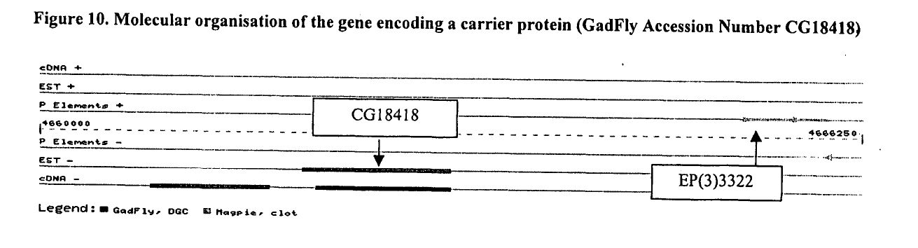

FIG. 10 shows the molecular organization of the mutated gene locus of Drosophila carrier protein (Gadfly Accession Number CG18418).

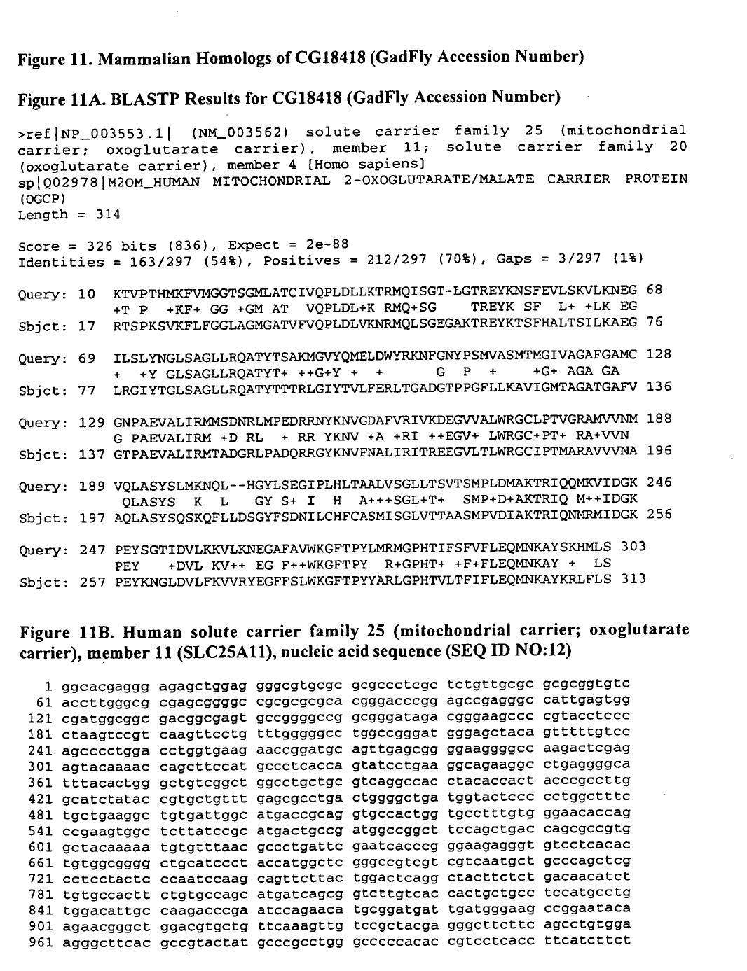

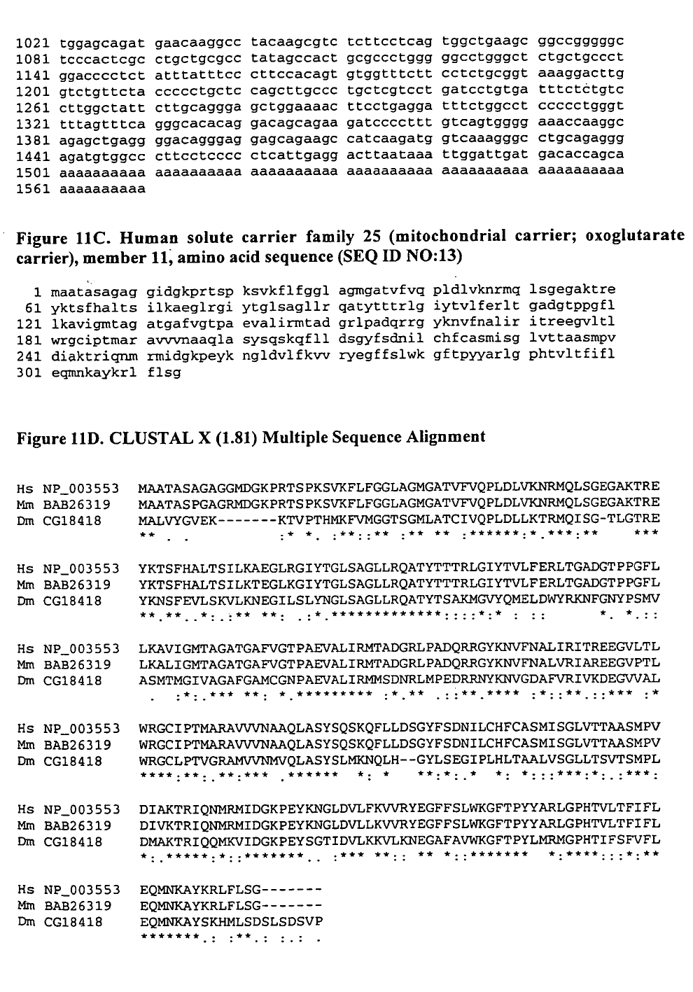

FIG. 11 shows the mammalian homologs of GadFly Accession Number CG18418.

FIG. 11A shows the BLASTP search result for CG18418 (Query) with the best human homologous match (Sbject).

FIG. 11B shows the nucleic acid sequence of human solute carrier family 25 (mitochondrial carrier; oxoglutarate carrier) member 11 (SEQ ID NO: 12; GenBank Accession Number NM_{—}003562).

FIG. 11C shows the amino acid sequence (one-letter code) of human solute carrier family 25 (mitochondrial carrier; oxoglutarate carrier) member 11 (SEQ ID NO: 13; GenBank Accession Number NP_{—}003553).

FIG. 11D shows the Clustal X (1.81) protein sequence alignment for human solute carrier (referred to as ‘Hs NP_{—}003553’), mouse putative solute carrier (referred to as ‘Mm BAB26319’), and Drosophila CG18418 (referred to as ‘Dm CG18418’). Gaps in the alignment are represented as

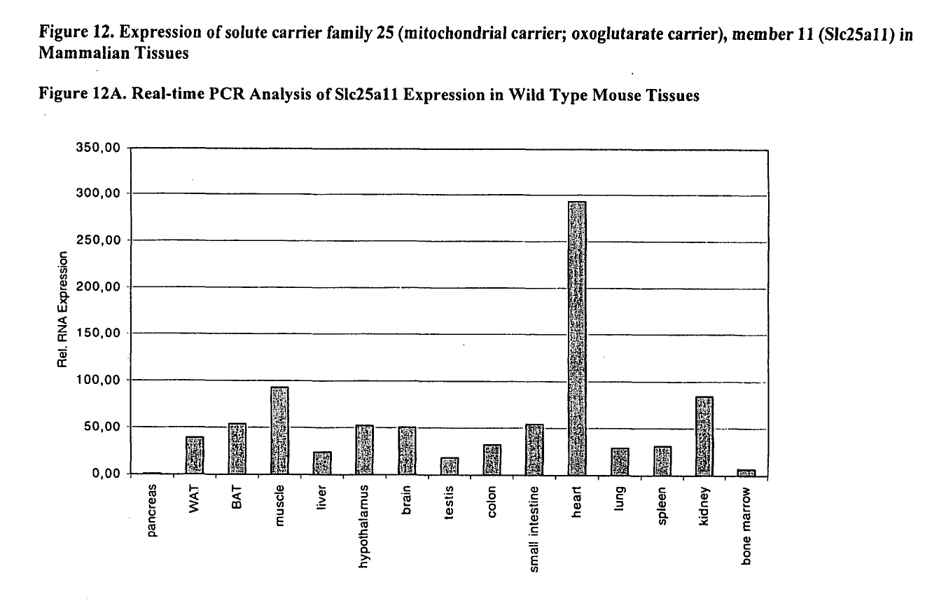

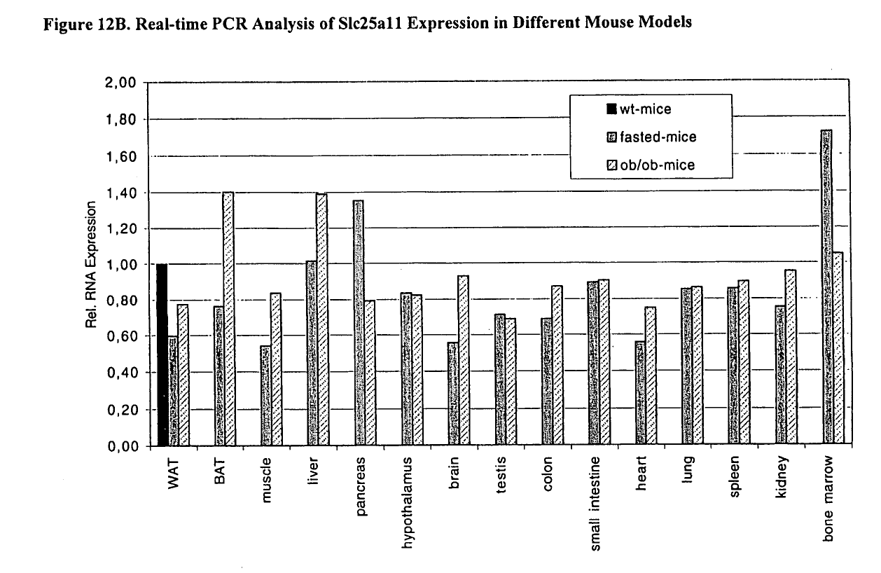

FIG. 12 shows the expression of solute carrier family 25 (mitochondrial carrier; oxoglutarate carrier), member 11 (Slc25a11) in mammalian tissues. The relative RNA-expression is shown on the Y-axis. In FIGS. 12A and B, the tissues tested are given on the X-axis. “WAT” refers to white adipose tissue, “BAT” refers to brown adipose tissue.

FIG. 12A: Real-time PCR analysis of Slc25a11 expression in wildtype mouse tissues.

FIG. 12B: Real-time PCR mediated analysis of Slc25al 1 expression in different mouse models.

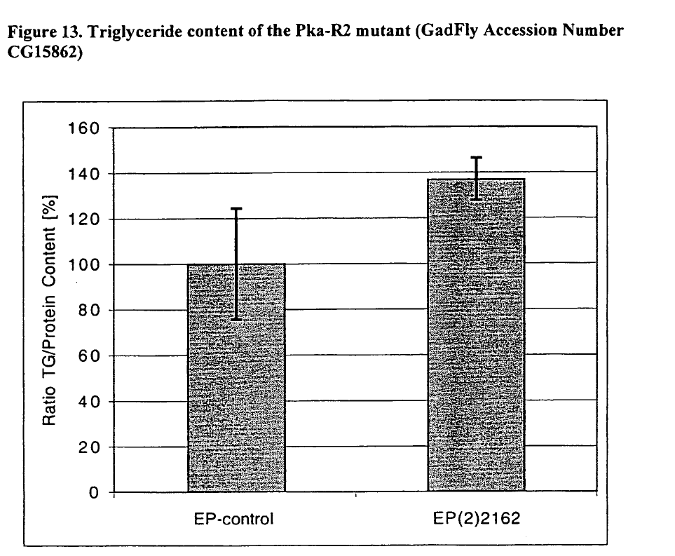

FIG. 13 shows the increase of triglyceride content of EP(2)2162 flies (referred to as ‘EP(2)2162’) caused by homozygous viable integration of the P-vector in comparison to controls with integration of these vectors elsewhere in the genome (referred to as ‘EP-control’).

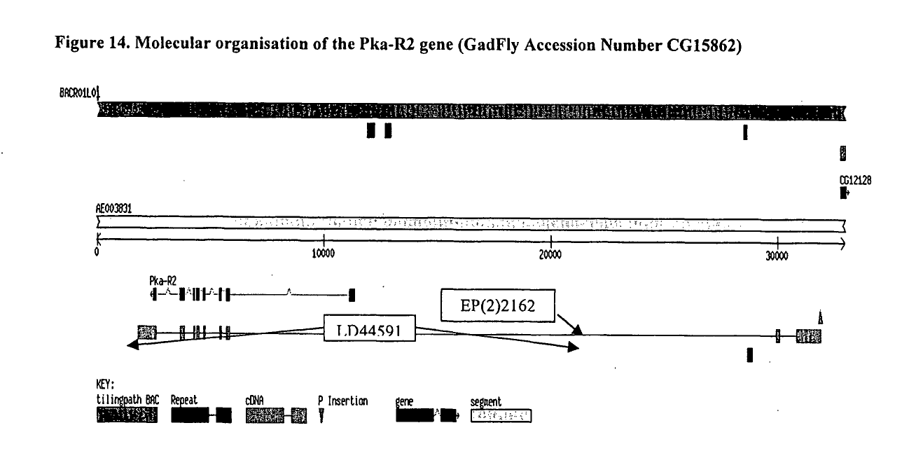

FIG. 14 shows the molecular organization of the mutated gene locus of Drosophila Pka-R2 (GadFly Accession Number CG15862).

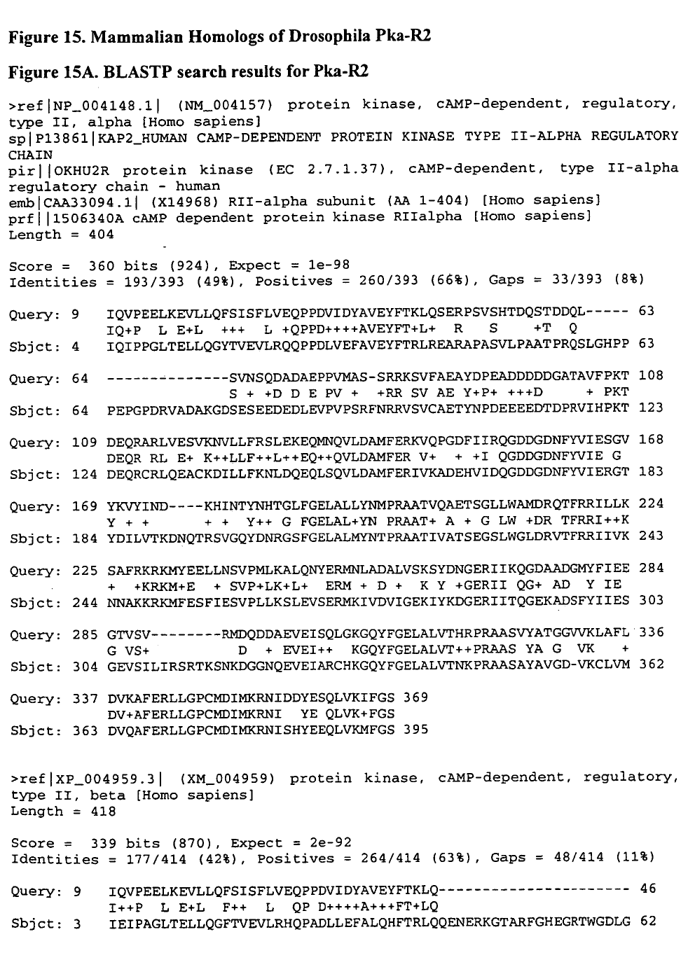

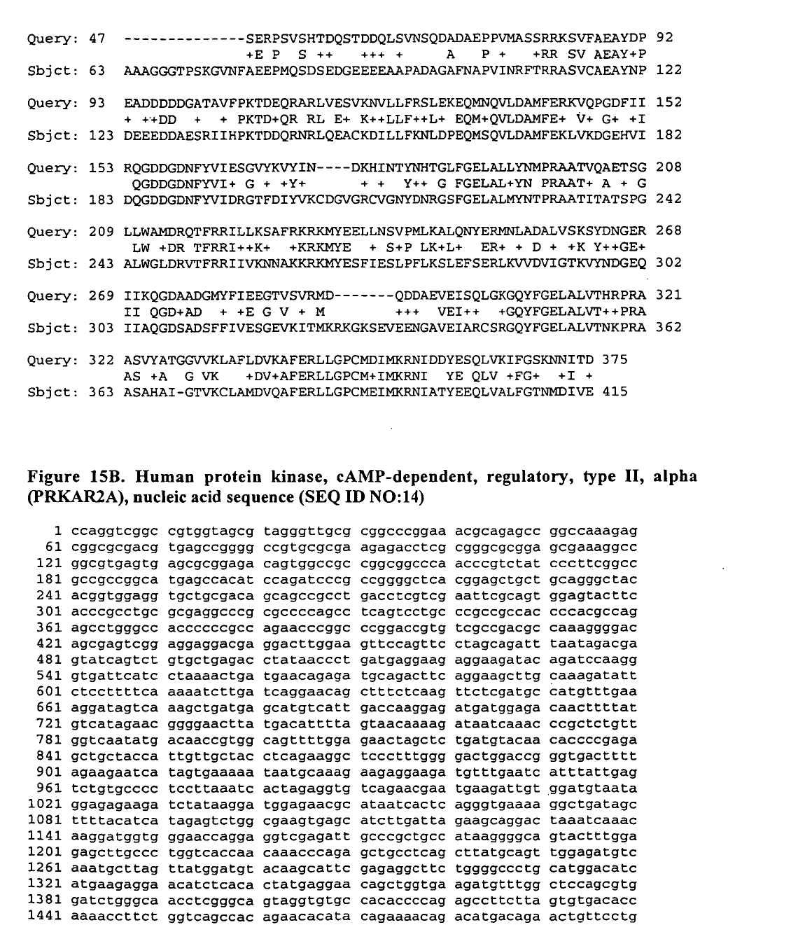

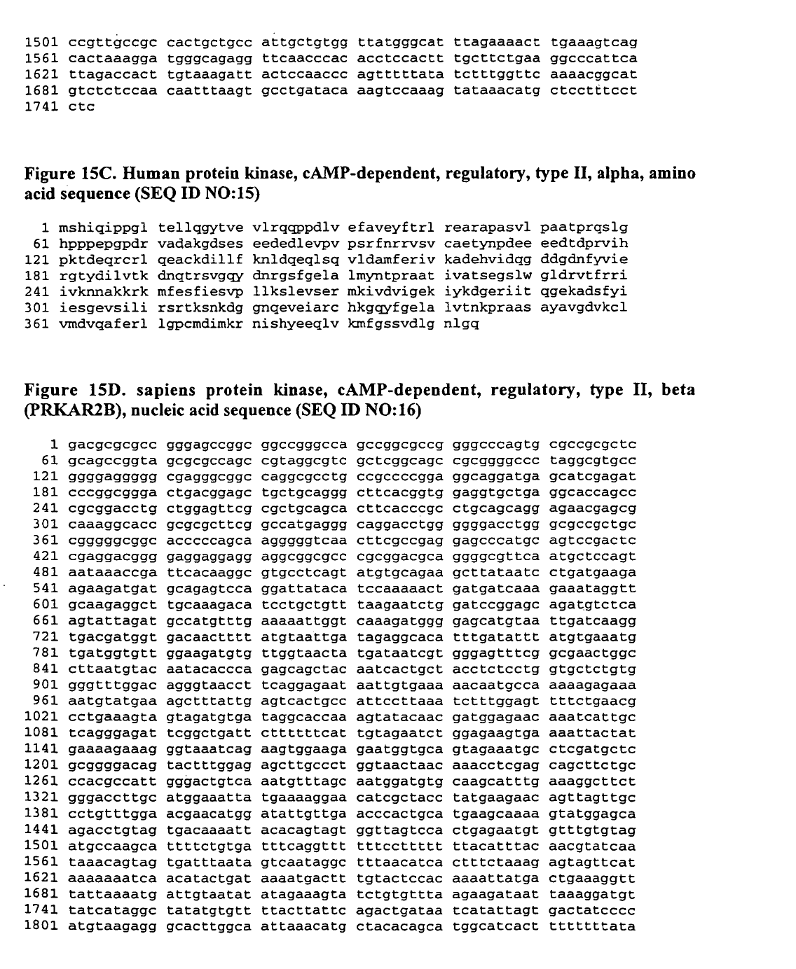

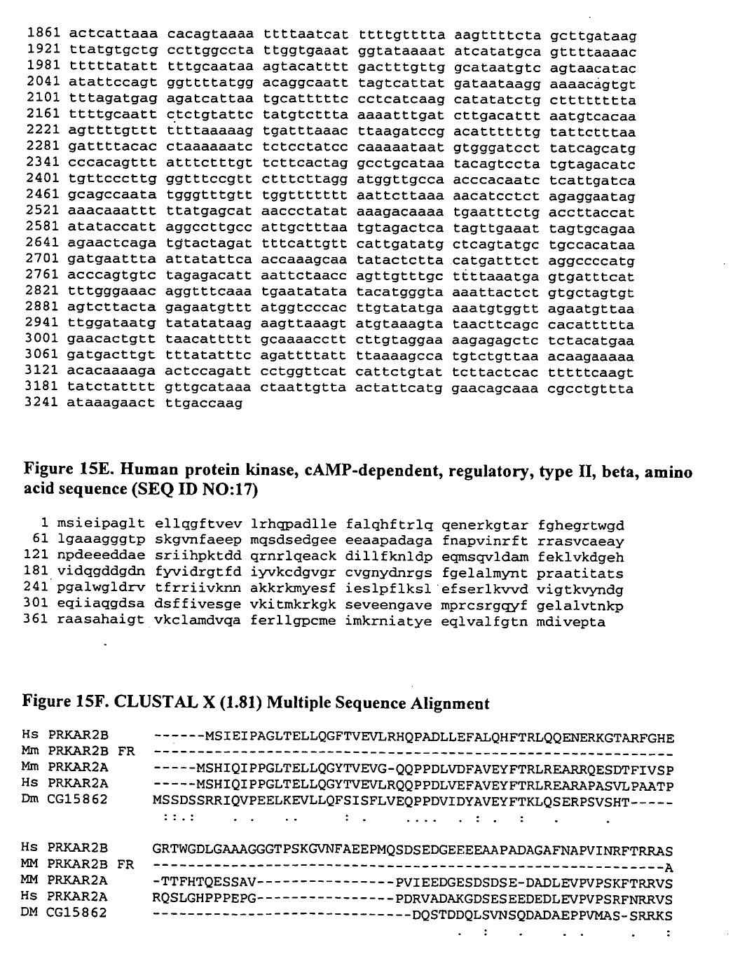

FIG. 15 shows the mammalian homologs of Drosophila Pka-R2.

FIG. 15A shows the BLASTP search result for Pka-R2 (Query), shown are only the human homologs with highest homology values (Sbjct).

FIG. 15B shows the nucleic acid sequence of human protein kinase, cAMP-dependent, regulatory, type II, alpha (SEQ ID NO: 14; GenBank Accession Number NM_{—}004157).

FIG. 15C shows the amino acid sequence (one-letter code) of human protein kinase, cAMP-dependent, regulatory, type II, alpha (SEQ ID NO: 15; GenBank Accession Number NP_{—}004148).

FIG. 15D shows the nucleic acid sequence of human protein kinase, cAMP-dependent, regulatory, type II, beta (SEQ ID NO: 16; GenBank Accession Number NM_{—}002736).

FIG. 15E shows the amino acid sequence (one-letter code) of human protein kinase, cAMP-dependent, regulatory, type II, beta (SEQ ID NO: 17; GenBank Accession Number NP_{—}002727).

FIG. 15F shows the Claustal X (1.81) multiple sequence alignment analysis of human PRKAR2B protein (referred to as ‘Hs PRKAR2B’), a fragment of murine PRKAR2B protein (referred to as ‘Mm PRKAR2B FR’), murine PRKAR2A protein (referred to as ‘Mm PRKAR2A’), human PRKAR2A protein (referred to as ‘Hs PRKAR2A’), and Drosophila Pka-R2 protein (referred to as ‘Dm CG15862’). Gaps in the alignment are represented as -.

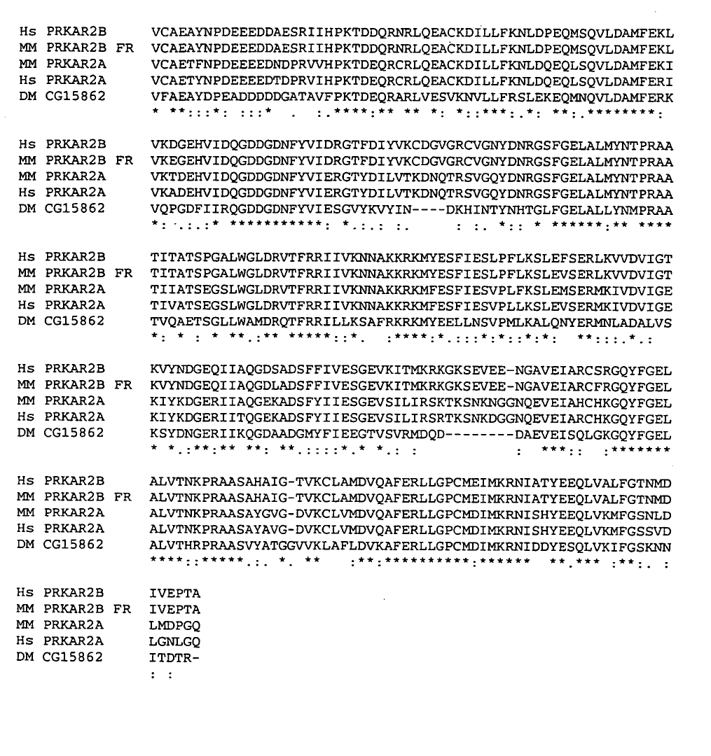

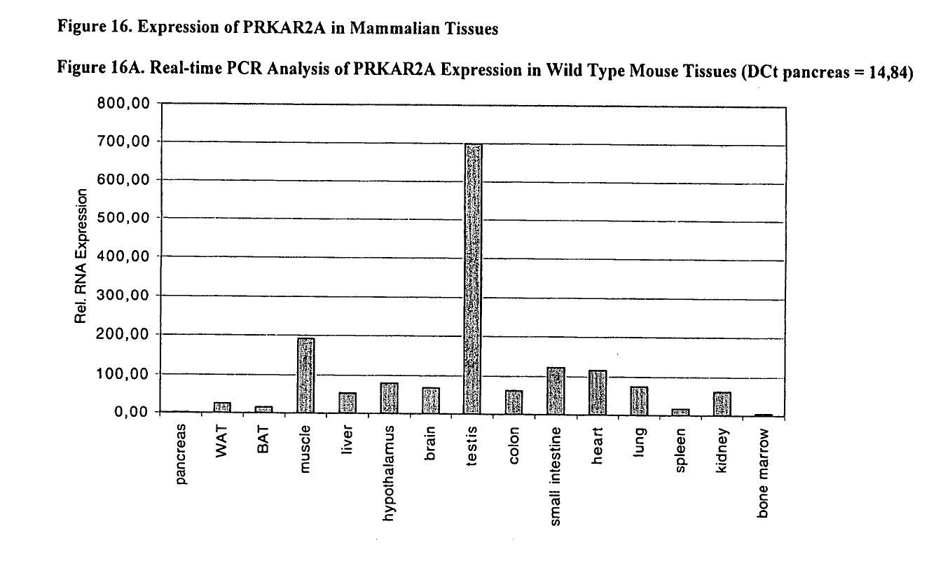

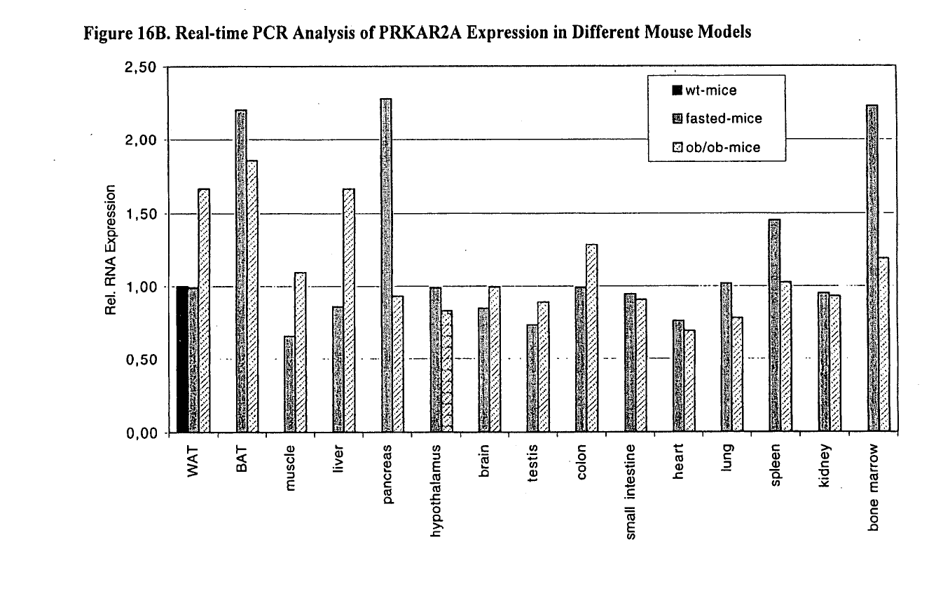

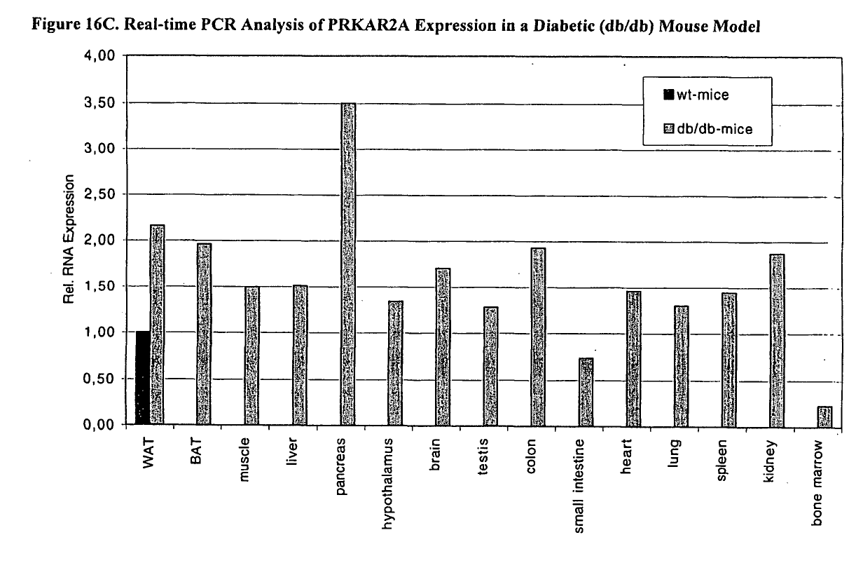

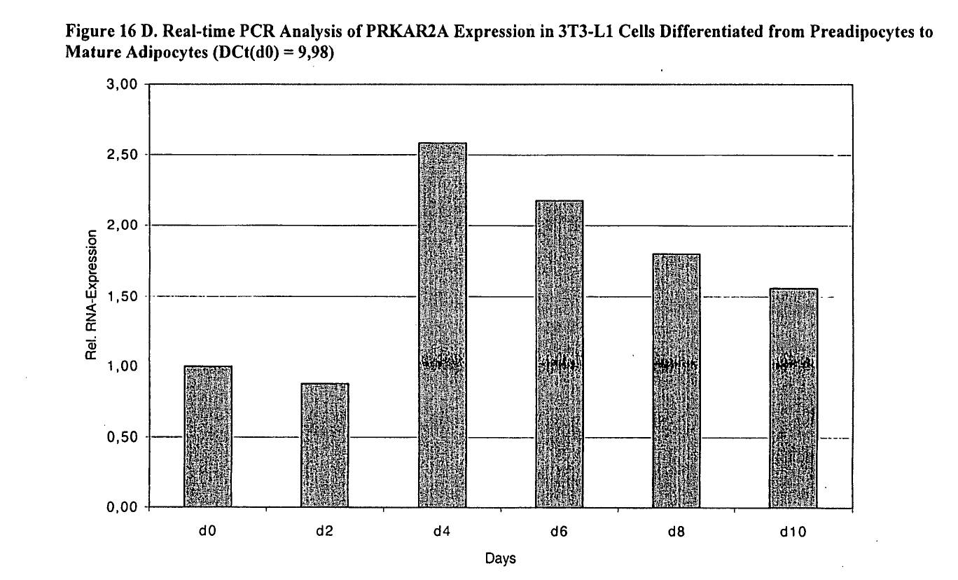

FIG. 16 shows the expression of PRKAR2A in mammalian tissues. The relative RNA-expression is shown on the Y-axis. In FIG. 16A, B, and C, the tissues tested are given on the X-axis. “WAT” refers to white adipose tissue, “BAT” refers to brown adipose tissue. In FIG. 16D, the X-axis represents the time axis. “d0” refers to day 0 (start of the experiment), “d2-“d10” refers to day 2-day 10 of adipocyte differentiation.

FIG. 16A: Real-time PCR analysis of PRKAR2A expression in wildtype mouse tissues.

FIG. 16B: Real-time PCR mediated analysis of PRKAR2A expression in different mouse models.

FIG. 16C: Real-time PCR mediated analysis of PRKAR2A expression in a diabetic (db/db) mouse model.

FIG. 16D: Real-time PCR mediated analysis of PRKAR2A expression during the differentiation of 3T3-L1 cells from preadipocytes to mature adipocytes.

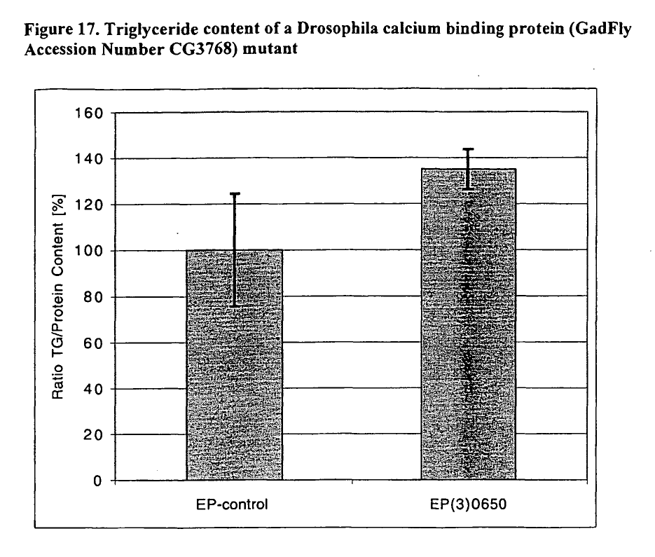

FIG. 17 shows the increase of triglyceride content of EP(3)0650 flies (referred to as ‘EP(3)0650’) caused by homozygous viable integration of the P-vector in comparison to controls with integration of these vectors elsewhere in the genome (referred to as ‘EP control’).

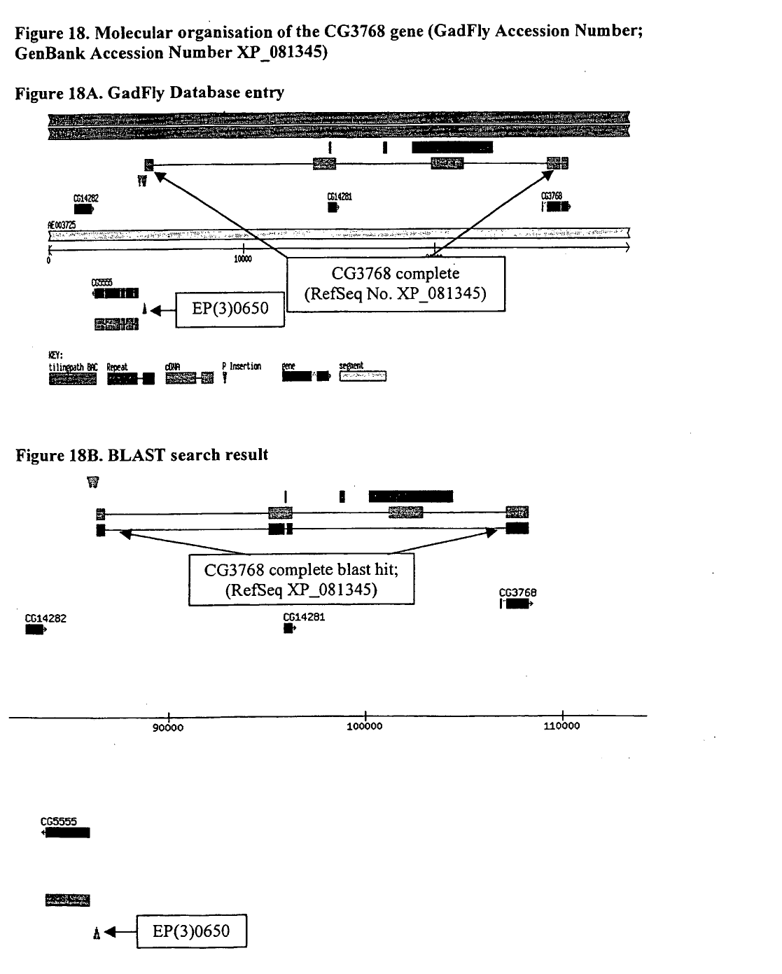

FIG. 18 shows the molecular organization of the mutated gene locus of GadFly Accession Number CG3768.

FIG. 18A shows the GadFly database entry for the mutated CG3768 gene locus.

FIG. 18B shows the BLAST search result for the mutated CG3768 gene locus.

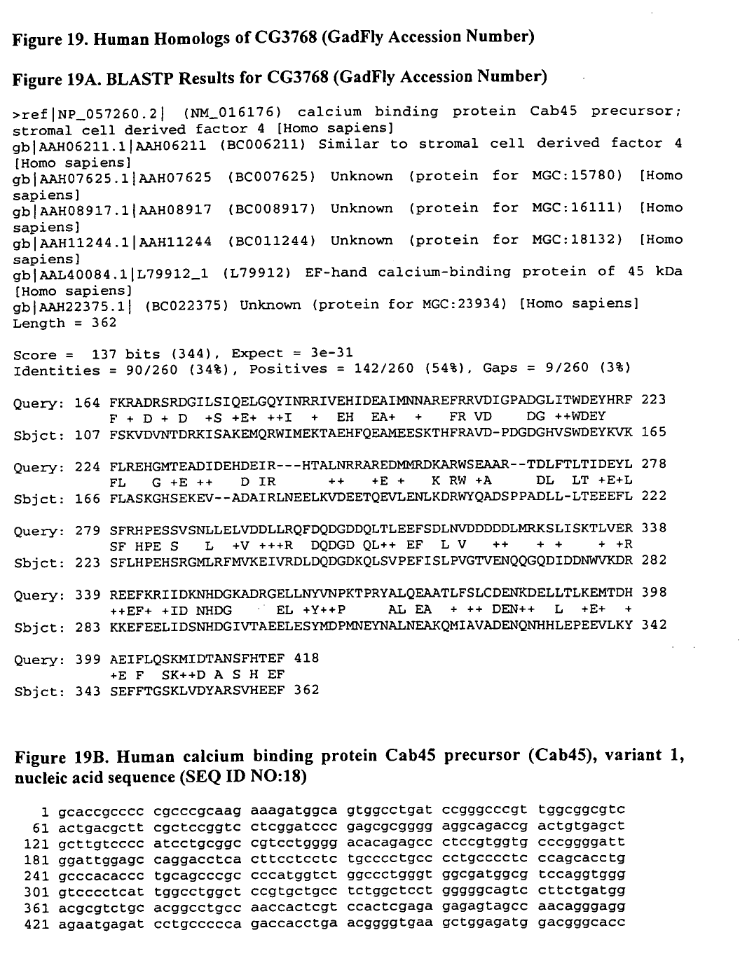

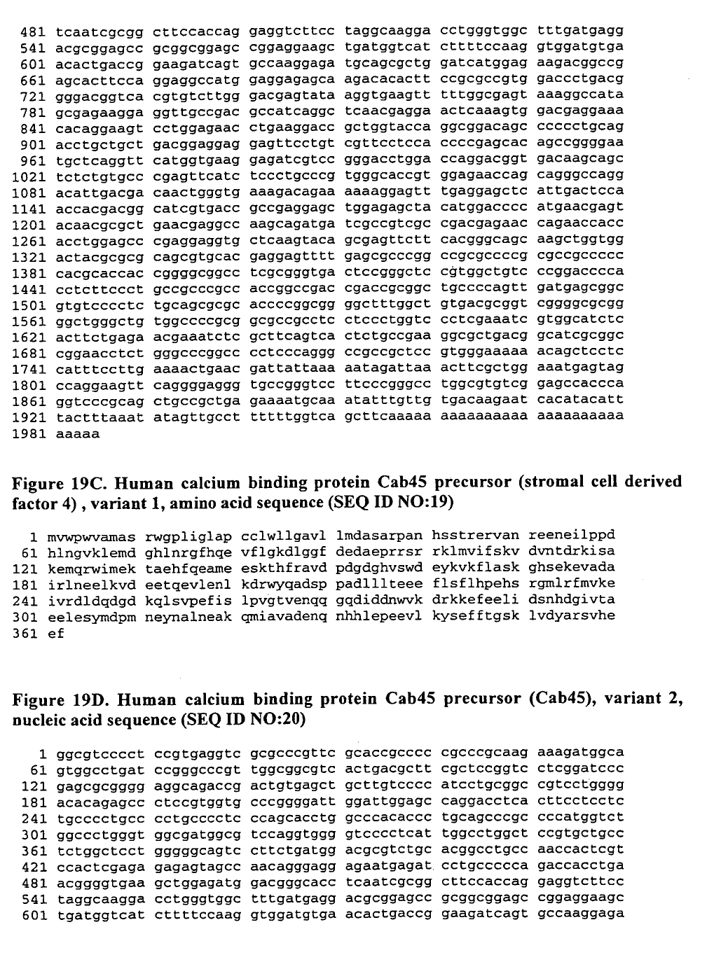

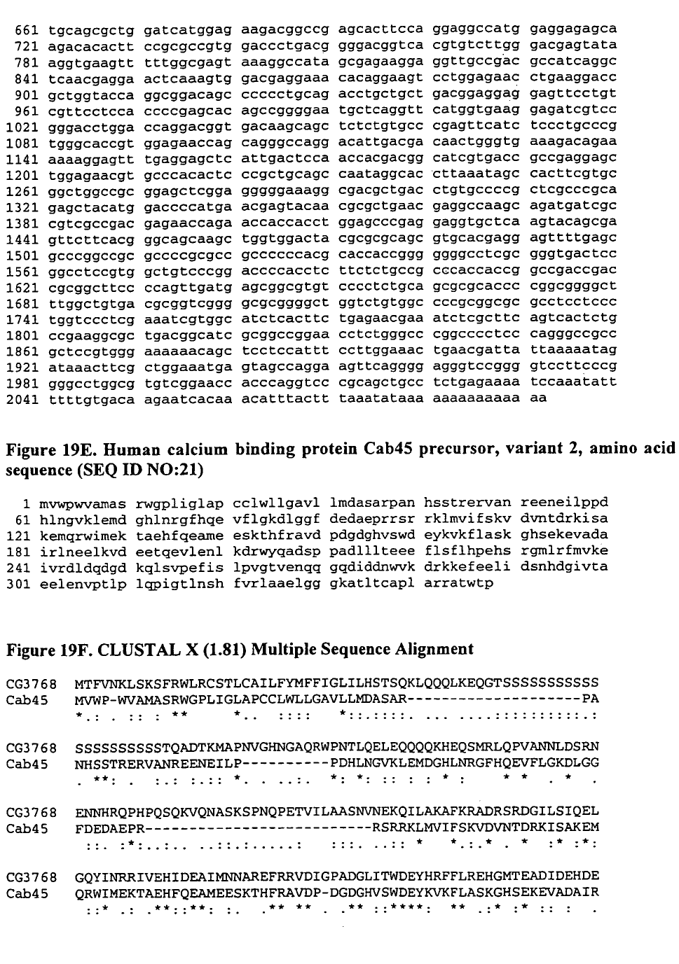

FIG. 19 shows the human homologs of GadFly Accession Number CG31475 (also referred to as Gadfly Accession Numbers CG14281 and CG3768).

FIG. 19A shows the BLASTP search result for CG31475 (Query) with the best human homologous match (Sbject).

FIG. 19B shows the nucleic acid sequence of human calcium binding protein Cab45 precursor variant 1 (SEQ ID NO: 18; GenBank Accession Number NM_{—}016176).

FIG. 19C shows the amino acid sequence (one-letter code) of human calcium binding protein Cab45 precursor variant 1 (SEQ ID NO: 19; GenBank Accession Number NP_{—}057260).

FIG. 19D shows the nucleic acid sequence of human calcium binding protein Cab45 precursor variant 2 (SEQ ID NO: 20; GenBank Accession Number NM_{—}016547).

FIG. 19E shows the amino acid sequence (one-letter code) of human calcium binding protein Cab45 precursor variant 2 (SEQ ID NO: 21; GenBank Accession Number NP_{—}057631).

FIG. 19F shows the Clustal X (1.81) protein alignment for the Drosophila protein encoded by GadFly Accession Number CG3768 (referred to as ‘CG3768’) and human Cab45 protein (GenBank Accession Number NP_{—}057260.2; referred to as ‘Cab45’).

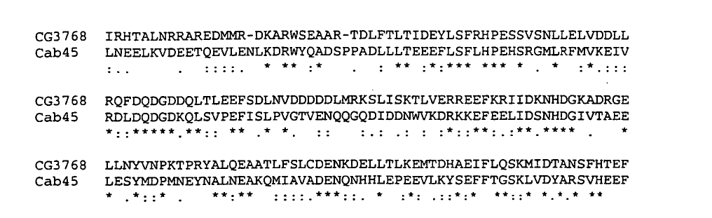

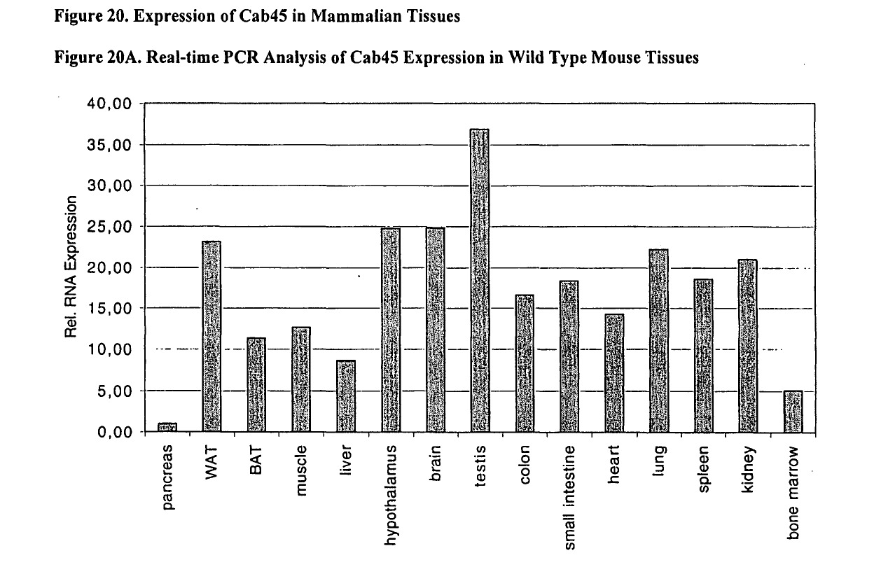

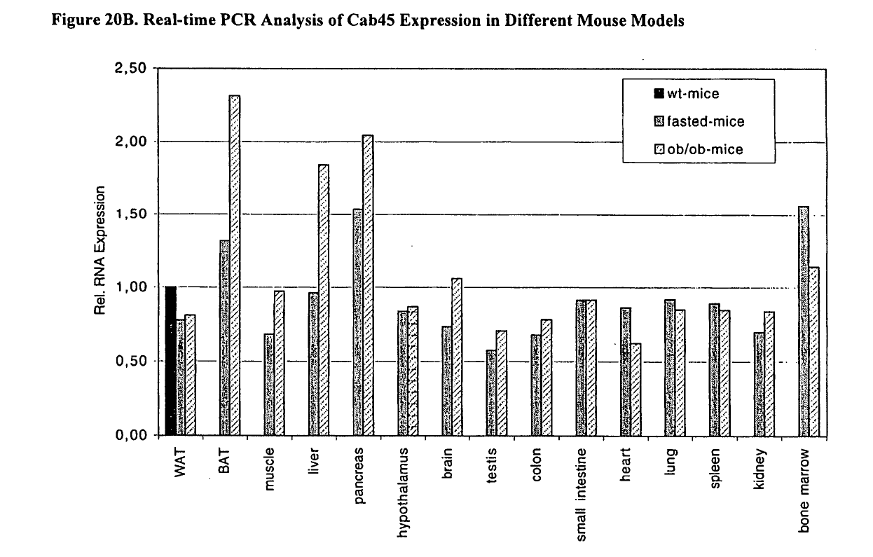

FIG. 20 shows the expression of Cab45 in mammalian tissues. The relative RNA-expression is shown on the Y-axis. In FIGS. 20A and B, the tissues tested are given on the X-axis. “WAT” refers to white adipose tissue, “BAT” refers to brown adipose tissue.

FIG. 20A: Real-time PCR analysis of Cab45 expression in wildtype mouse tissues.

FIG. 20B: Real-time PCR mediated analysis of Cab45 expression in different mouse models.

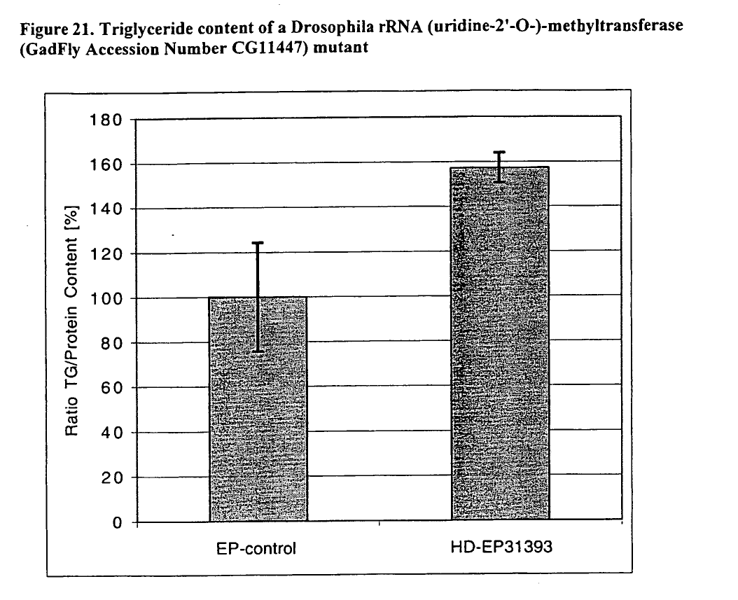

FIG. 21 shows the triglyceride content of a Drosophila rRNA (uridine-2′-O-)-methyl-transferase protein (GadFly Accession Number CG11447) mutant. Shown is the increase of triglyceride content of HD-EP(3)31393 flies (referred to as ‘HD-EP31393’) caused by homozygous viable integration of the P-vector in comparison to controls with integration of these vectors elsewhere in the genome (referred to as ‘EP control’).

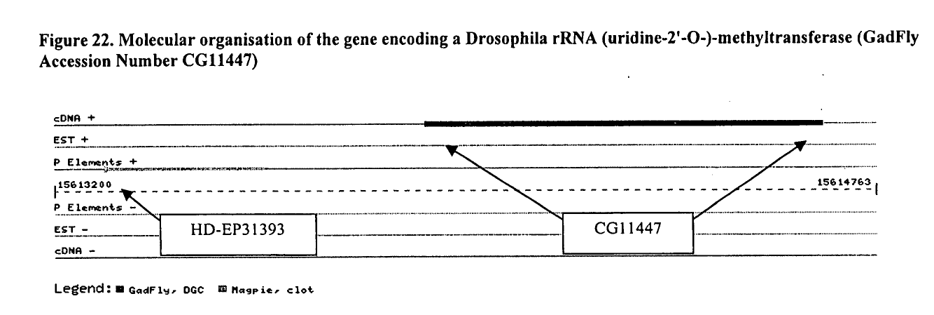

FIG. 22 shows the molecular organization of the mutated gene locus of Drosophila rRNA (uridine-2′-O-)-methyltransferase (Gadfly Accession Number CG11447).

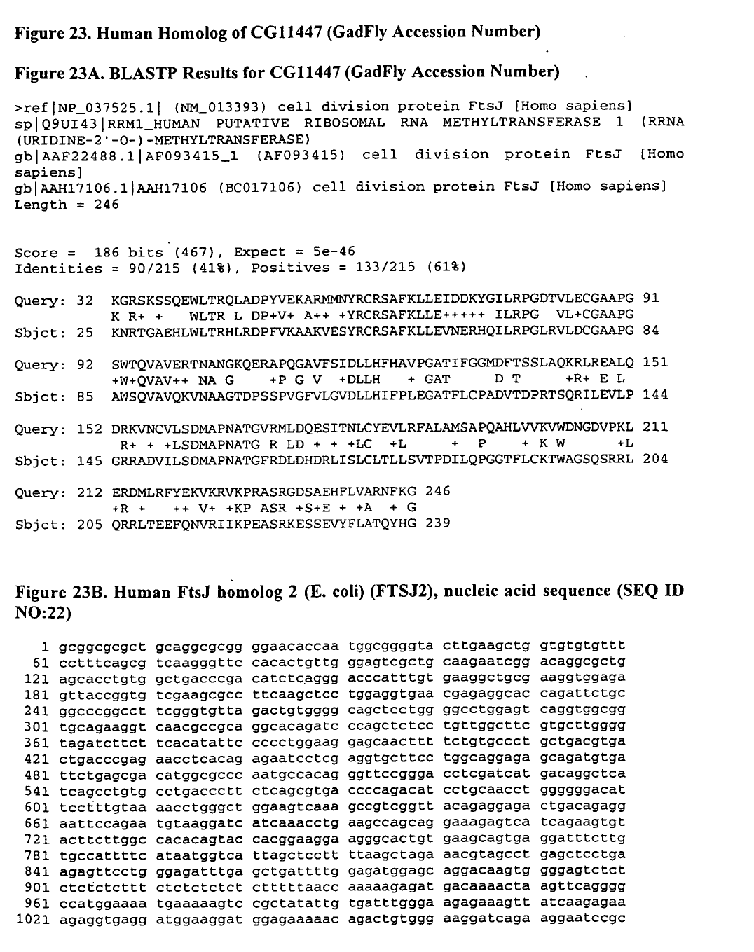

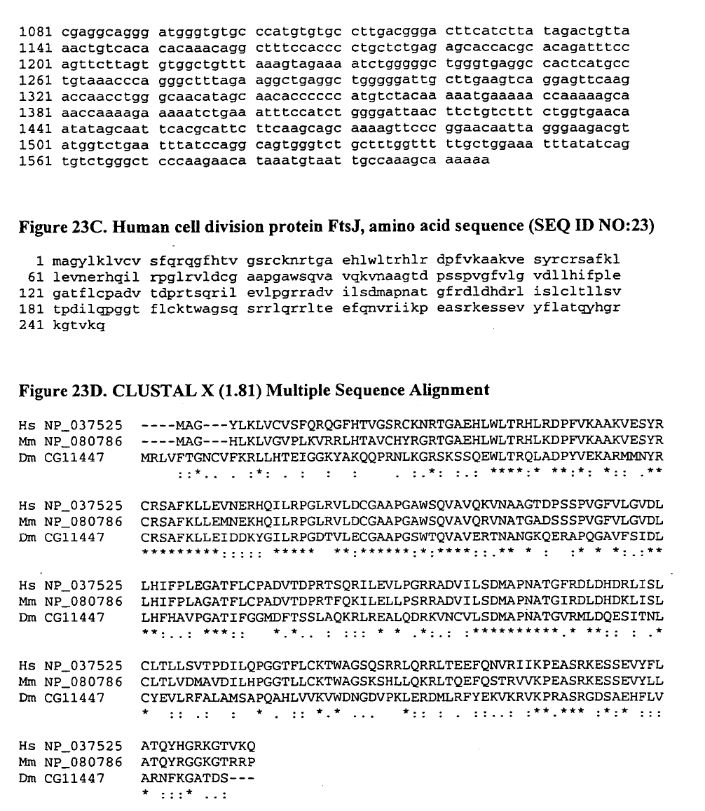

FIG. 23 shows the mammalian homologs of GadFly Accession Number CG 1447.

FIG. 23A shows the BLASTP search result for CG 11447 (Query) with the best human homologous match (Sbject).

FIG. 23B shows the nucleic acid sequence of human cell division protein FtsJ (SEQ ID NO: 22; GenBank Accession Number NM_{—}013393).

FIG. 23C shows the amino acid sequence (one-letter code) of human cell division protein FtsJ (SEQ ID NO: 23; GenBank Accession Number NP_{—}037525).

FIG. 23D shows the Clustal X (1.81) protein sequence alignment for human ribosomal RNA (rRNA) methyltransferase (referred to as ‘Hs NP_{—}037525’), murine rRNA methyltransferase (referred to as ‘Mm NP_{—}080786’), and Drosophila rRNA methyltransferase (referred to as ‘Dm CG11447’).

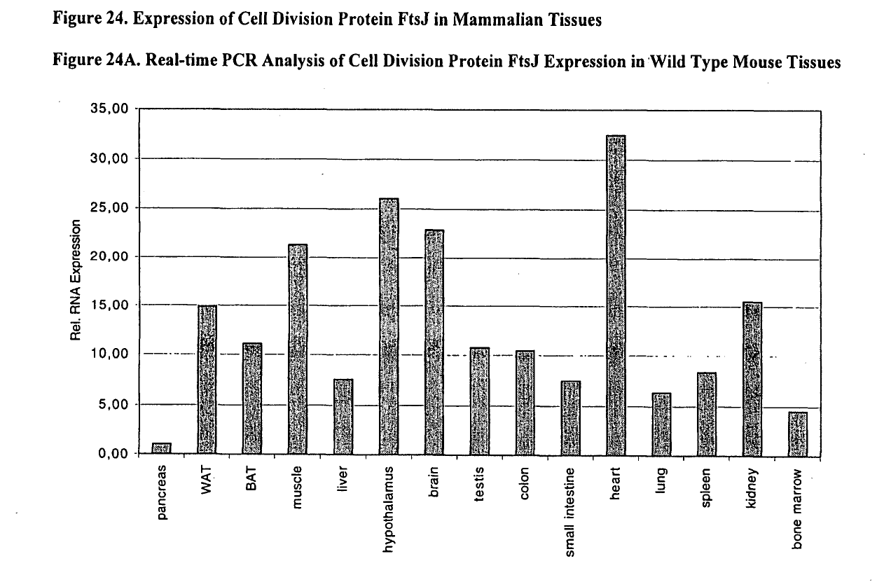

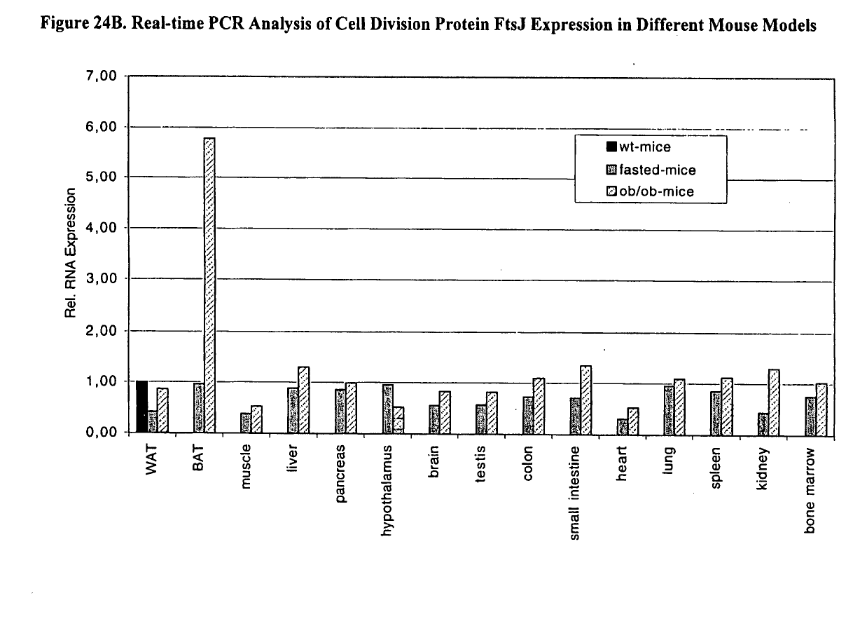

FIG. 24 shows the expression of cell division cycle protein FtsJ in mammalian tissues. The relative RNA-expression is shown on the Y-axis. In FIG. 24A and B, the tissues tested are given on the X-axis. “WAT” refers to white adipose tissue, “BAT” refers to brown adipose tissue.

FIG. 24A: Real-time PCR analysis of cell division cycle protein FtsJ expression in wildtype mouse tissues.

FIG. 24B: Real-time PCR mediated analysis of cell division cycle protein FtsJ expression in different mouse models.

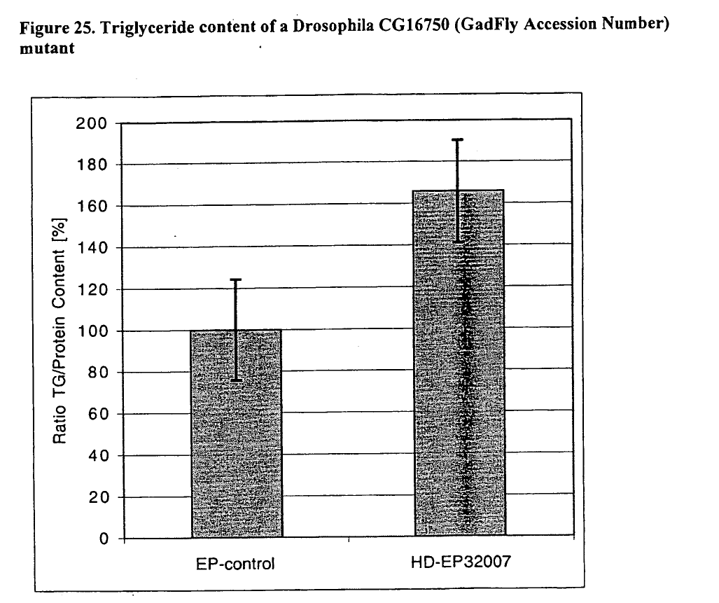

FIG. 25 shows the increase of triglyceride content of HD-EP(3)2007 flies (referred to as ‘HD-EP32007’) caused by homozygous viable integration of the P-vector in comparison to controls with integration of these vectors elsewhere in the genome (referred to as ‘EP control’).

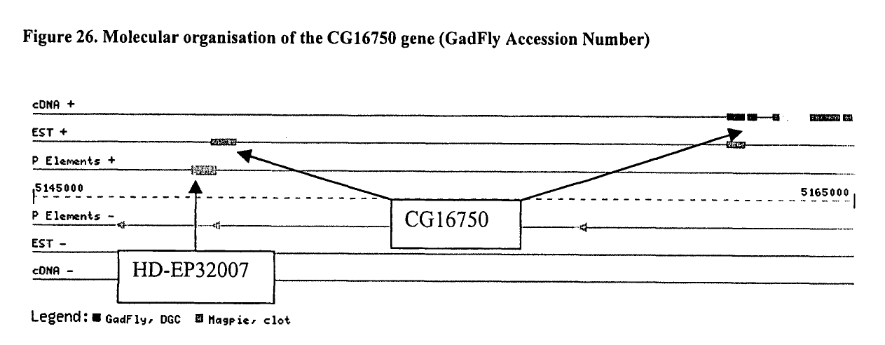

FIG. 26 shows the molecular organization of the mutated gene locus of GadFly Accession Number CG16750.

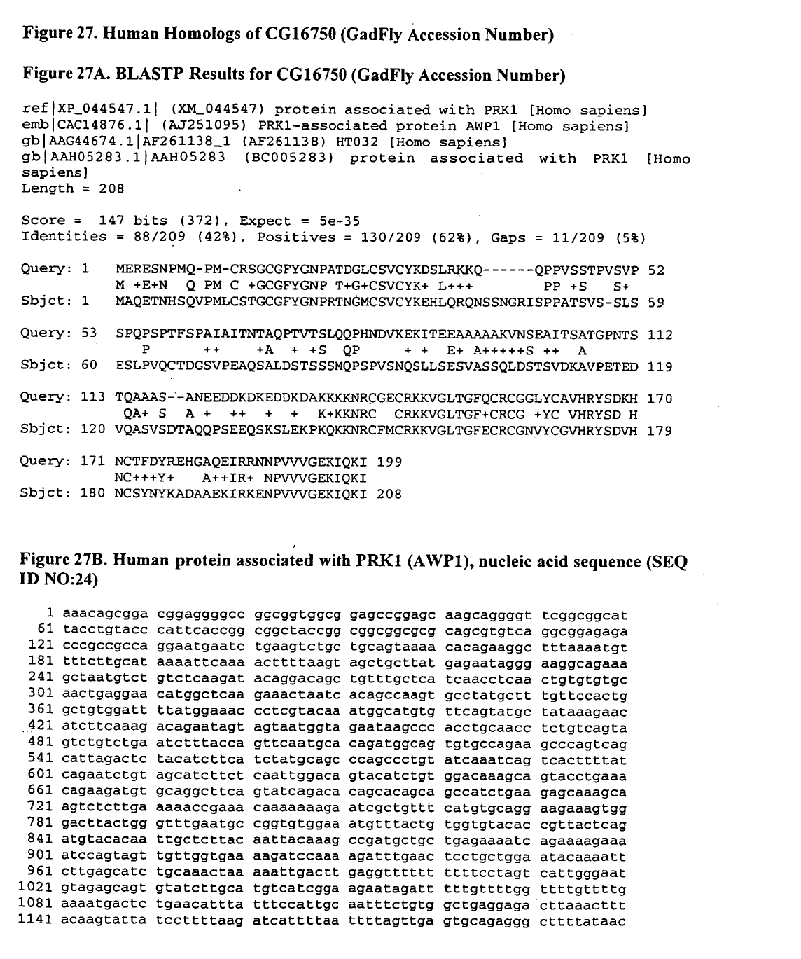

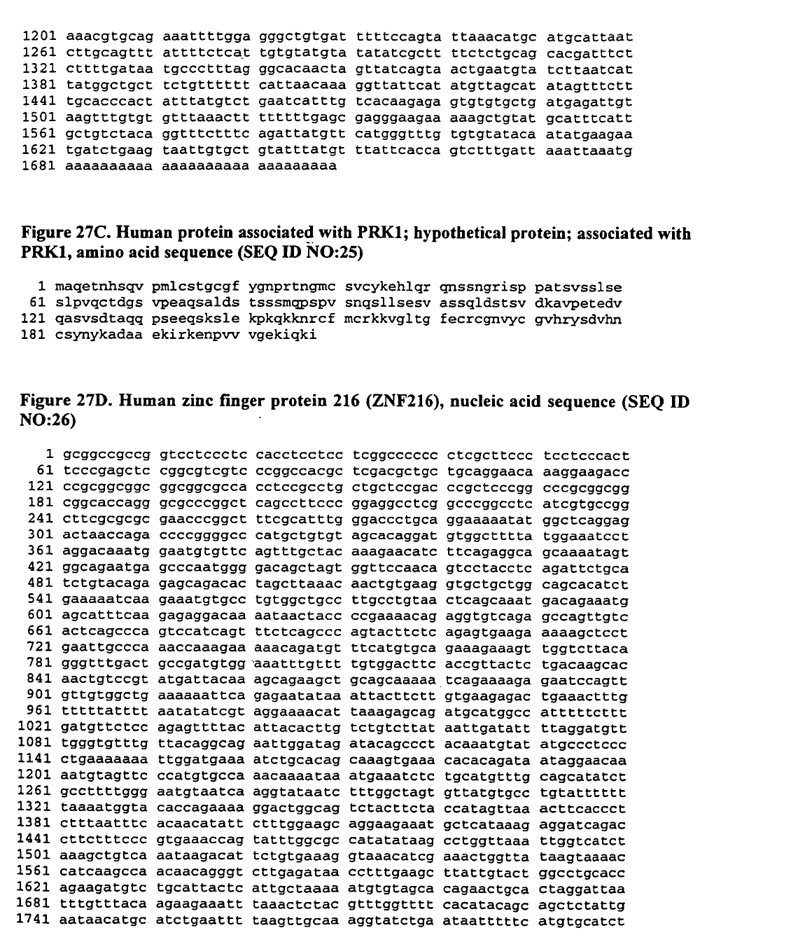

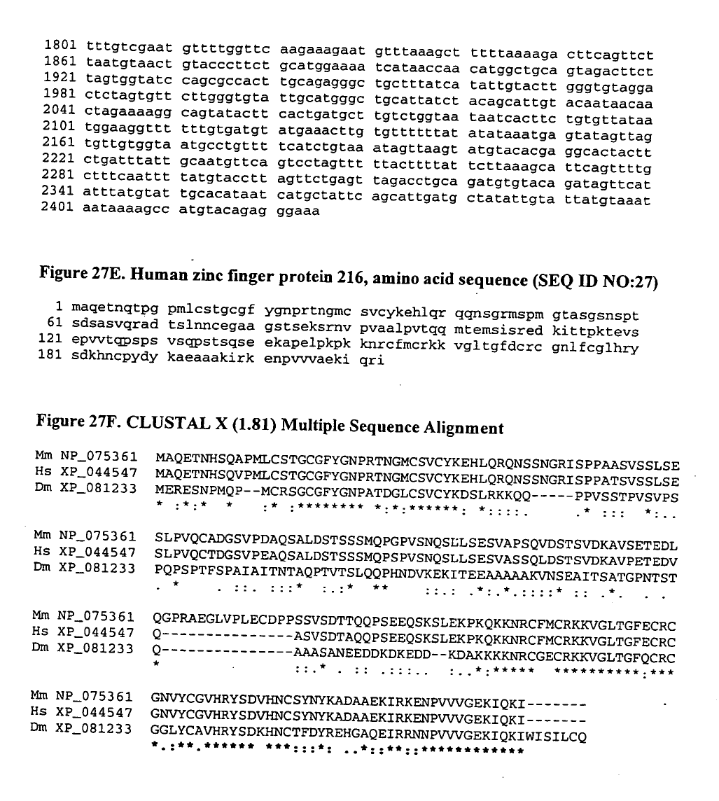

FIG. 27 shows the human homologs of GadFly Accession Number CG16750.

FIG. 27A shows the BLASTP search result for CG16750 (Query) with the best human homologous match (Sbject).

FIG. 27B shows the nucleic acid sequence of human protein associated with PKR1 (SEQ ID NO: 24; GenBank Accession Number NM_{—}019006).

FIG. 27C shows the amino acid sequence (one-letter code) of human protein associated with PKR1 (SEQ ID NO: 25; GenBank Accession Number NP_{—}061879).

FIG. 27D shows the nucleic acid sequence of human zinc finger protein 216 (SEQ ID NO: 26; GenBank Accession Number NM_{—}006007).

FIG. 27E shows the amino acid sequence (one-letter code) of human neuropeptide zinc finger protein 216 (SEQ ID NO: 27; GenBank Accession Number NP_{—}005998).

FIG. 27F shows the Clustal X (1.81) protein alignment for the mouse protein NP_{—}075361.2 (referred to as ‘Mm NP_{—}075361’), human AWP1 protein (referred to as ‘Hs XP_{—}044547’), and Drosophila protein encoded by GadFly Accession Number CG16750 (referred to as ‘Dm XP_{—}0081233’).

The examples illustrate the invention:

### EXAMPLE 1

### Measurement of Triglyceride Content

Mutant flies are obtained from a proprietary fly mutation stock collection and a publicly availabla stock collection. The flies are grown under standard conditions known to those skilled in the art. In the course of the experiment, additional feedings with bakers yeast (Saccharomyces cerevisiae) are provided. The average increase of triglyceride content of Drosophila containing the EP-vectors in homozygous or hemizygous viable integration was investigated in comparison to control flies (see FIGS. 1, 5, 9, 13, 17, 21, and 25). For determination of triglyceride, flies were incubated for 5 min at 90° C. in an aqueous buffer using a waterbath, followed by hot extraction. After another 5 min incubation at 90° C. and mild centrifugation, the triglyceride content of the flies extract was determined using Sigma Triglyceride (INT 336-10 or -20) assay by measuring changes in the optical density according to the manufacturer's protocol. As a reference protein content of the same extract was measured using BIO-RAD DC Protein Assay according to the manufacturer's protocol. The assay was repeated three times.

The average triglyceride level of all flies of the EP collections (referred to as ‘EP-control’) is shown as 100% in the first columns in FIGS. 1, 5, 9, 1 3, 17, 21, and 25, including standard deviation. EP(3)3054 homozygous flies show constantly a higher triglyceride content than the control flies tested (column 2 in FIG. 1). Therefore, the loss of gene activity in the locus 85E4 on chromosome 3R where the EP-vector of EP(3)3054 flies is homozygous viable integrated, is responsible for changes in the metabolism of the energy storage triglycerides.

It was found in this invention that homozygous HD-EP(3)31068 and HD-EP(3)31149 flies show constantly a higher triglyceride content than the control flies tested (columns 2 and 3 in FIG. 5). Therefore, the loss of gene activity in the locus 93D4-5 on chromosome 3R where the EP-vectors of HD-EP(3)31068 and HD-EP(3)31149 flies are homozygous viable integrated, is responsible for changes in the metabolism of the energy storage triglycerides.

EP(3)3322 homozygous flies show constantly a higher triglyceride content than the controls (column 2 in FIG. 9). Therefore, the loss of gene activity in the locus 64C1 on chromosome 3L where the EP-vector of EP(3)3322 flies is homozygous viable integrated, is responsible for changes in the metabolism of the energy storage triglycerides.

EP(2)2162 homozygous flies show constantly a higher triglyceride content than the controls (column 2 in FIG. 13). Therefore, the loss of gene activity in the locus 46D1-3 on chromosome 2R where the EP-vector of EP(2)2162 flies is homozygous viable integrated, is responsible for changes in the metabolism of the energy storage triglycerides.

EP(3)0650 homozygous flies show constantly a higher triglyceride content than the controls (column 2 in FIG. 17). Therefore, the loss of gene activity in the locus 91F8 on chromosome 3R where the EP-vector of EP(3)0650 flies is homozygous viable integrated, is responsible for changes in the metabolism of the energy storage triglycerides. HD-EP(3)31393 homozygous flies show constantly a higher triglyceride content than the controls (column 2 in FIG. 21). Therefore, the loss of gene activity in the locus 92B6 on chromosome 3R where the EP-vector of HD-EP(3)31393 flies is homozygous viable integrated, is responsible for changes in the metabolism of the energy storage triglycerides.

EP(3)32007 homozygous flies show constantly a higher triglyceride content than the controls (column 2 in FIG. 25). Therefore, the loss of gene activity in the locus 85D11 on chromosome 3R where the EP-vector of HD-EP(3)32007 flies is homozygous viable integrated, is responsible for changes in the metabolism of the energy storage triglycerides.

### EXAMPLE 2

### Identification of Drosophila Genes Associated With Metabolic Control

Nucleic acids encoding the proteins of the present invention were identified using a plasmid-rescue technique. Genomic DNA sequences were isolated that are localized adjacent to the EP vector (herein EP(3)3054, HD-EP(3)31068, HD-EP(3)31149, EP(3)3322, EP(2)2162, EP(3)0650, HD-EP(3)31393, and HD-EP(3)32007) integration. Using those isolated genomic sequences public databases like Berkeley Drosophila Genome Project (GadFly) were screened thereby identifying the integration sites of the vectors, and the corresponding genes. The molecular organization of these gene loci is shown in FIGS. 2, 6, 10, 14, 18, 22, and 26.

In FIG. 2, genomic DNA sequence is represented by the assembly as a scaled black line in the middle (numbers represent the length in basepairs of the genomic DNA) that includes the integration site of vector for line EP(3)3054. A transcribed DNA sequence (EST) and predicted genes are shown as bars on the upper side (sense strand). Predicted exons of the cDNA with GadFly Accession Number CG8327 are shown as dark grey bars and introns as light grey lines. The sequence encodes for a gene that is predicted by GadFly sequence analysis programs as Accession Number CG8327. Public DNA sequence databases (for example, NCBI GenBank) were screened thereby identifying the integration site of line EP(3)3054, causing an increase of triglyceride content. EP(3)3054 is integrated into the first cDNA in antisense orientation of the cDNA with Accession Number CG8327 (the site of integration is shown as triangle in the lower half of the Figure). Therefore, expression of the cDNA encoding Accession Number CG8327 could be effected by homozygous viable integration of vectors of line EP(3)3054, leading to increase of the energy storage triglycerides.

The HD-EP(3)31068 and the HD-EP(3)31149 vectors are homozygous viable integrated into the enhancer region of a Drosophila gene in 5′ antisense and sense orientation, respectively. The gene was identified as Berkeley Drosophila Genome Project Accession No. CG10823. FIG. 6 shows the molecular organization of this gene locus. The chromosomal localization site of the integration of the vector of HD-EP(3)31068 and HD-EP(3)31149 is at gene locus 3R, 93D4-5. In FIG. 6, genomic DNA sequence is represented as a black dotted line in the middle that includes the integration sites of HD-EP(3)31068 and HD-EP(3)31149. Numbers represent the coordinates of the genomic DNA (starting at position 17023997 on chromosome 3R, ending at position 17048997 on chromosome 3R). The upper half of the Figure represents the sense orientation (+), the lower half represents the antisense orientation (−). Grey bars on the two “cDNA”-lines represent the predicted genes (as predicted by the Berkeley Drosophila Genome Project, GadFly and by Magpie). Predicted exons of the Drosophila cDNA (Berkeley Drosophila Genome Project Accession Nr. CG10823) are shown as dark grey bars and predicted introns as light grey bars. Grey bars on the lower “EST”-line represent transcribed DNA. The identified cDNA sequence encodes for a gene that is predicted by GadFly sequence analysis programs as Accession Number CG10823. Public DNA sequence databases (for example, NCBI or GenBank) were screened thereby identifying the integration sites of lines HD-EP(3)31068 and HD-EP(3)31149, causing an increase of triglyceride content. Therefore, expression of the cDNA encoding Accession Number CG10823 could be effected by homozygous integration of vectors of line HD-EP(3)31068 and HD-EP(3)31149, leading to increase of the energy storage triglycerides.

In FIG. 10, genomic DNA sequence is represented by the assembly as a dotted black line (from position 4660000 to 4666250 on chromosome 3L) that includes the integration sites of vector for line EP(3)3322. Transcribed DNA sequences (ESTs) and predicted exons are shown as bars in the lower two lines. Predicted exon of the cDNA with GadFly Accession Number CG18418 is shown as dark grey bar. CG18418 encodes for a gene that is predicted by GadFly sequence analysis programs as Accession Number CG18418 (see for example, sequence 190 from patent WO0140519). Public DNA sequence databases (for example, NCBI GenBank) were screened thereby identifying the integration sites of lines EP(3)3322, causing an increase of triglyceride content. EP(3)3322 is integrated into the enhancer region of CG18418 in antisense orientation of the cDNA with Accession Number CG18418. Therefore, expression of the cDNA encoding Accession Number CG18418 could be effected by homozygous integration of vectors of line EP(3)3322, leading to increase of the energy storage triglycerides.

In FIG. 14, genomic DNA sequence is represented as a black thin scaled line in the middle that includes the integration site of EP(2)2162. Numbers represent the length in base pairs of the genomic DNA. Black bars on the lower side represent the predicted genes (as predicted by the Berkeley Drosophila Genome Project, GadFly and by Magpie). Predicted exons of the Drosophila cDNA (Berkeley Drosophila Genome Project Accession No. CG15862) are shown as black bars and predicted introns as linking light grey lines. Light grey bars on the lower half of the figure represent transcribed DNA. The symbols are described in more detail in the key at the bottom of FIG. 14. Pka-R2 encodes for a gene that is predicted by GadFly sequence analysis programs as Accession Number CG15862. Public DNA sequence databases (for example, NCBI GenBank) were screened thereby identifying the integration sites of lines EP(2)2162, causing an increase of triglyceride content. EP(2)2162 is integrated into the promoter in sense direction of the cDNA with Accession Number CG15862. Therefore, expression of the cDNA encoding Accession Number CG15862 could be effected by homozygous integration of vectors of line EP(2)2162, leading to increase of the energy storage triglycerides.

In FIGS. 18A and B, genomic DNA sequence is represented as a thin black bar in the middle that includes the integration site of EP(3)0650. Numbers represent the length in base pairs of the genomic DNA. The upper black bars in FIGS. 18A and B represent repetitive sequences, the grey bars below represent cDNA sequences. Grey triangles represent P-insertions, black bars, labeled with CG-numbers represent genes. In FIG. 18A, the genes CG14281 and CG3768 are annotated incorrect. In FIG. 18B the corresponding BLAST search result is shown. According to the BLAST result, the gene with GadFly Accession Number CG3768 (GenBank Accession Number XP_{—}081345) extends more in 5prime direction. In FIGS. 18A and B predicted exons of the Drosophila cDNA (Berkeley Drosophila Genome Project Accession Nr. CG3768) are shown as black bars and predicted introns as black lines.

In FIG. 22, genomic DNA sequence is represented by the assembly as a dotted black line (from position 15613200 to 15614763 on chromosome 3R) that includes the integration sites of vector for line HD-EP(3)31393. A predicted gene is shown as bar in the topmost line. A predicted exon of the cDNA with GadFly Accession Number CG11447 is shown as dark grey bar. The sequence encodes for a gene that is predicted by GadFly sequence analysis programs as Accession Number CG11447. Public DNA sequence databases (for example, NCBI GenBank) were screened thereby identifying the integration sites of lines HD-EP(3)31393, causing an increase of triglyceride content. HD-EP(3)31393 is integrated into the enhancer region in sense orientation of the cDNA with Accession Number CG11447. Therefore, expression of the cDNA encoding Accession Number CG11447 could be effected by homozygous integration of vectors of line HD-EP(3)31393, leading to increase of the energy storage triglycerides.

In FIG. 26, genomic DNA sequence is represented by the assembly as a dotted black line (from position 5145000 to 5165000 on chromosome 3R) that includes the integration sites of vector for line EP(3)32007. Transcribed DNA sequences (ESTs) and predicted exons are shown as bars in the upper two lines. Predicted exons of the cDNA with GadFly Accession Number CG16750 are shown as dark grey bars and introns as light grey bars. Public DNA sequence databases (for example, NCBI GenBank) were screened thereby identifying the integration sites of lines EP(3)32007, causing an increase of triglyceride content. EP(3)32007 is integrated into the promoter of CG16750 in sense orientation of the cDNA with Accession Number CG16750. Therefore, expression of the cDNA encoding Accession Number CG16750 could be effected by homozygous integration of vectors of line EP(3)32007, leading to increase of the energy storage triglycerides.

### EXAMPLE 3

### Identification of Human Homologous Genes and Proteins

The Drosophila genes and proteins encoded thereby with functions in the regulation of triglyceride metabolism were further analysed using the BLAST algorithm searching in publicly available sequence databases and mammalian homologs were identified (see FIGS. 3, 7, 11, 15, 19, 23, and 27).

As shown in FIG. 3A, the gene product of Drosophila CG8327 is 76% homologous over 283 amino acids (of 302 amino acids) to a human spermidine synthase (also referred to as spermidine synthase-1; SRM; GenBank Accession Number NM_{—}003132 for the cDNA, NP_{—}003123 for the protein). Furthermore the gene product of Drosophila CG8327 is 76% homologous over 283 amino acids (of 302 amino acids) to murine spermidine synthase (GenBank Accession Number NM_{—}009272 for the cDNA, NP_{—}033298 for the protein). In FIGS. 3B and 3C, the nucleic acid and amino acid sequences of the human spermidine synthase are shown. FIG. 3D shows a Clustal X (1.81) protein alignment of spermidine synthase homologs of human, mouse, and Drosophila.

CG10823 homologous proteins and nucleic acid molecules coding therefore are obtainable from insect or vertebrate species, e.g. mammals or birds. Particularly preferred are nucleic acids encoding a Drosophila CG10823 protein (GadFly Accession Number CG10823), a G protein-coupled receptor 74 (GPR74, neuropeptide FF2 receptor; Genbank Accession Number NM_{—}053036 for the cDNA, NP_{—}444264.1 for the protein), neuropeptide FF1 receptor (Genbank Accession Number NM_{—}022146 for the cDNA, NP_{—}071429.1 for the protein), orexin receptor 1 (OX1R; GenBank Accession Number NM_{—}001525 for the cDNA, NP_{—}001516 for the protein), and orexin receptor 2 (OX2R; GenBank Accession Number NM_{—}001526for the cDNA, NP_{—}001517 for the protein). A BLASTP search for GadFly Accession Number CG10823 (SEQ ID NO: 3; see FIG. 7A) was conducted. The best human match is neuropeptide FF2 receptor (NP_{—}444264; 38% identity, 58% homology, data not shown), the second best human match is neuropeptide FF1 receptor (NP_{—}071429; 34% identity, 52% homology, data not shown). In FIG. 7B, C, D, E, F, G, H, and I, the nucleic acid and amino acid sequences of the human homologs of CG10823 are shown. An alignment of CG10823 homologous proteins from different species has been done by the Clustal W multiple sequence alignment program and is shown in FIG. 3J.

CG18418 homologous proteins and nucleic acid molecules coding therefore are obtainable from insect or vertebrate species, e.g. mammals or birds. Particularly preferred are nucleic acids comprising GadFly Accession Number CG18418 and a human solute carrier family 25 (mitochondrial carrier; oxoglutarate carrier) member 11 protein (SLC25A11; GenBank Accession Number NM_{—}003562 for the cDNA, NP_{—}003553 for the protein). As shown in FIG. 11A, gene product of GadFly Accession Number CG18418 (which is identical to sequence 190 from patent application WO0140519; GenBank Accession Number AX154841.1) is 70% homologous over 297 amino acids (of 314 amino acids) to human SLC25A11 (GenBank Accession Number NM_{—}003562; lacobazzi, V. et al, 1992, DNA Seq. 3(2):79-88). CG18418 also shows 69% homology on protein level over 279 amino acids (of 314 amino acids) to mouse solute carrier family 25 (mitochondrial carrier; oxoglutarate carrier), member 11 protein (GenBank Accession Number BAB2631 9.1). In FIGS. 11B and C, the nucleic acid and amino acid sequences of the human homologs of CG18418 are shown. FIG. 11D shows a Clustal X (1.81) protein alignment of the solute carrier family homologs of human (NP_{—}003553), mouse (BAB26319), and Drosophila(CG18418).

Pka-R2 homologous proteins and nucleic acid molecules coding therefore are obtainable from insect or vertebrate species, e.g. mammals or birds. Particularly preferred are nucleic acids encoding a Drosophila Pka-R2 protein (GadFly Accession Number CG1 5862), regulatory subunit RII alpha of human cAMP-dependent protein kinase (PRKAR2A; Genbank Accession Number NM_{—}004157 for the cDNA, NP_{—}004148.1 for the protein), or regulatory subunit RII beta of human cAMP-dependent protein kinase (PRKAR2B; GenBank Accession Number NM_{—}002736 for the cDNA, NP_{—}002727 for the protein, formerly Genbank Accession Number XM_{—}004959 for the cDNA, XP_{—}004959.3 for the protein). The BLASTP search results for GadFly Accession Number CG1 5862 (Pka-R2) are shown in FIG. 15A; shown are only the two human homologous proteins with highest identity values. In FIGS. 15B, C, D, and E, the nucleic acid and amino acid sequences of the human homologs of Pka-R2 are shown. An alignment of Pka-R2 from different species has been done by the Clustal X multiple sequence alignment program and is shown in FIG. 15F.

CG3768 homologous proteins and nucleic acid molecules coding therefore are obtainable from insect or vertebrate species, e.g. mammals or birds.

Particularly preferred are nucleic acids comprising GadFly Accession Number CG3768 (GenBank Accession Number XP_{—}081345), and human calcium binding protein Cab45 precursor (Cab45; GenBank Accession Numbers NM_{—}016176 and NM_{—}016547 for the cDNAs, NP_{—}057260 and NP_{—}057631 for the proteins). As shown in FIG. 19A, the gene product of Drosophila CG3768 (GenBank Accession Number XP_{—}081345) is 54% homologous over 260 amino acids (of 362 amino acids) to human calcium binding protein Cab45 precursor (also referred to as Cab45; GenBank Accession Number NP_{—}057260.2; Scherer P. E. et al., 1996, J Cell Biol 133(2):257-268). The gene product of Drosophila CG3768 is 53% homologous over 261 amino acids (of 361 amino acids) to mouse calcium-binding protein Cab45b (GenBank Accession Number AAB01813.1). In FIGS. 19B, C, D, and E, the nucleic acid and amino acid sequences of the human homologs of CG3768 are shown. FIG. 19F shows a Clustal X (1.81) protein alignment of the Cab45 homologs of Drosophila (CG3768) and human (Cab45).

Methyl transferase homologous proteins and nucleic acid molecules coding therefore are obtainable from insect or vertebrate species, e.g. mammals or birds. Particularly preferred are nucleic acids encoding GadFly Accession Number CG11447, human cell division protein FtsJ (FtsJ2; GenBank Accession Number NM_{—}013393 for the CDNA, NP_{—}037525 for the protein; Sequence 72 from Patent WO0107471, GenBank Accession Number AX078268). As shown in FIG. 23A, gene product of GadFly Accession Number CG11447 is 61% homologous over 215 amino acids (of 246 amino acids) to human cell division protein FtsJ (GenBank Accession Number NM_{—}013393). Additionally the gene product of GadFly Accession Number CG11447 is 63% homologous over 215 amino acids (of 246 amino acids) to mouse rRNA methyltransferase (RIKEN cDNA 2310037B18; GenBank Accession Number NM_{—}026510 for the cDNA, NP_{—}080786 for the protein). In FIGS. 23B and C, the nucleic acid and amino acid sequences of the human homologs of CG11447 are shown. FIG. 23D shows a Clustal X (1.81) protein alignment of methyltransferase homologs of human, mouse, and Drosophila.

CG16750 homologous proteins and nucleic acid molecules coding therefore are obtainable from insect-or vertebrate species, e.g. mammals or birds. Particularly preferred are nucleic acids comprising GadFly Accession Number CG16750, human protein associated with PRK1 (AWP1; GenBank Accession Number NM_{—}019006 for the cDNA, NP_{—}061879 for the protei, formerly GenBank Accession Number XP_{—}044547.1), and human zinc finger protein 216 (ZNF216, GenBank Accession Number NM_{—}006007 for the cDNA, NP_{—}005998 for the protein). As shown in FIG. 27A, gene product of GadFly Accession Number CG1 6750 is 62% homologous over 209 amino acids (of 208 amino acids) to human protein associated with PRK1 (AWP1; GenBank Accession Number XP_{—}044547.1). The gene product of GadFly Accession Number CG16750 is 55% homologous over 227 amino acids (of 223 amino acids) to mouse protein associated with Prkcl1 (GenBank Accession Number NP_{—}075361.2). In FIGS. 27B, C, D, and E, the nucleic acid and amino acid sequences of the human homologs of CG16750 are shown. FIG. 27F shows a Clustal X (1.81) protein alignment of the homologs of mouse (Mm NP_{—}075361), human (Hs XP_{—}044547), and Drosophila (Dm XP_{—}081233).

### EXAMPLE 4

### Expression Profiling Experiments

To analyze the expression of the polypeptides disclosed in this invention in mammalian tissues, several mouse strains (preferrably mouse strains C57BI/6J, C57BI/6 ob/ob and C57BI/KS db/db which are standard model systems in obesity and diabetes research) were purchased from Harlan Winkelmann (33178 Borchen, Germany) and maintained under constant temperature (preferrably 22° C.), 40 per cent humidity and a light/dark cycle of preferrably 14/10 hours. The mice were fed a standard diet (for example, from ssniff Spezialitäten GmbH, order number ssniff M-Z V1126-000). For the fasting experiment (“fasted-mice”), wild type mice were starved for 48 h without food, but only water supplied ad libitum (see, for example, Schnetzler et al. J Clin Invest 1993 July;92(1):272-80, Mizuno et al. Proc Natl Acad Sci USA Apr. 16, 1996;93(8):3434-8). Animals were sacrificed at an age of 6 to 8 weeks. The animal tissues were isolated according to standard procedures known to those skilled in the art, snap frozen in liquid nitrogen and stored at −80° C. until needed.

For analyzing the role of the proteins disclosed in this invention in the in vitro differentiation of different mammalian cell culture cells for the conversion of pre-adipocytes to adipocytes, mammalian fibroblast (3T3-L1) cells (e.g., Green & Kehinde, Cell 1: 113-116, 1974) were obtained from the American Tissue Culture Collection (ATCC, Hanassas, Va., USA; ATCC-CL 173). 3T3-L1 cells were maintained as fibroblasts and differentiated into adipocytes as described in the prior art (e.g., Qiu. et al., J. Biol. Chem. 276:11988-95, 2001; Slieker et al., BBRC 251: 225-9, 1998). At various time points of the differentiation procedure, beginning with day 0 (day of confluence) and day 2 (hormone addition; for example, dexamethasone and 3-isobutyl-1-methylxanthine), up to 10 days of differentiation, suitable aliquots of cells were taken every two days. Alternatively, mammalian fibroblast 3T3-F442A cells (e.g., Green & Kehinde, Cell 7: 105-113, 1976) were obtained from the Harvard Medical School, Department of Cell Biology (Boston, Mass., USA). 3T3-F442A cells were maintained as fibroblasts and differentiated into adipocytes as described previously (Djian, P. et al., J. Cell. Physiol., 124:554-556, 1985). At various time points of the differentiation procedure, beginning with day 0 (day of confluence and hormone addition, for example, Insulin), up to 10 days of differentiation, suitable aliquots of cells were taken every two days. 3T3-F442A cells are differentiating in vitro already in the confluent stage after hormone (insulin) addition.

RNA was isolated from mouse tissues or cell culture cells using Trizol Reagent (for example, from Invitrogen, Karlsruhe, Germany) and further purified with the RNeasy Kit (for example, from Qiagen, Germany) in combination with an DNase-treatment according to the instructions of the manufacturers and as known to those skilled in the art. Total RNA was reverse transcribed (preferrably using Superscript II RNaseH-Reverse Transcriptase, from Invitrogen, Karlsruhe, Germany) and subjected to Taqman analysis preferrably using the Taqman 2×PCR Master Mix (from Applied Biosystems, Weiterstadt, Germany; the Mix contains according to the Manufacturer for example AmpliTaq Gold DNA Polymerase, AmpErase UNG, dNTPs with dUTP, passive reference Rox and optimized buffer components) on a GeneAmp 5700 Sequence Detection System (from Applied Biosystems, Weiterstadt, Germany).

Taqman analysis of spermidine synthase (Srm) was performed preferrably using the following primer/probe pairs: mouse Srm forward primer (Seq ID NO: 28) 5′-CCG TGC CGC CTT CGT AC-3′; mouse Srm reverse primer (Seq ID NO: 29) 5′-ACC TGG ATT CAG CTT ATG TCA TTG-3′; mouse Srm Taqman probe (Seq ID NO: 30) (5/6-FAM) CCT GAG TTC ACC CGG AAG GCC C (5/6-TAMRA).

Taqman analysis of RFamide-related peptide receptor was performed preferrably using the following primer/probe pairs: mouse RFamide-related peptide receptor forward primer (Seq ID NO: 31) 5′ GCA GCT GCA CTT GCT GTC C-3′; mouse RFamide-related peptide receptor reverse primer (Seq ID NO: 32) 5′-TGC TGT GGA AGA AGG CCA G-3′; mouse RFamide-related peptide receptor Taqman probe (Seq ID NO: 33) (5/6-FAM) TCT ACG CCT TCC CGT TGG CAC ACT (5/6-TAMRA).

Taqman analysis of G protein-coupled receptor 74 (Gpr74) was performed preferrably using the following primer/probe pairs: mouse Gpr74 forward primer (Seq ID NO: 34) 5′-TGA GAG CGA AAC GCA ACA TG-3′; mouse Gpr74 reverse primer (Seq ID NO: 35) 5′-GTT TTG AGA CAC CGG TTC CTG-3′; mouse Gpr74 Taqman probe (Seq ID NO: 36) (5/6-FAM) TCA TAA ACA CAT CGG GCC TGC TGG T (5/6-TAMRA).

Taqman analysis of the solute carrier family 25 (mitochondrial carrier; oxoglutarate carrier), member 11 (Slc25al 1) was performed preferrably using the following primer/probe pairs: mouse Slc25a11 forward primer (Seq ID NO: 37) 5′-CAT CCC TAC CAT GGC TCG AG-3′; mouse Slc25a11 reverse primer (Seq ID NO: 38) 5′-TGC TTA GAT TGA GAG TAA GAG GCA AG-3′; mouse Slc25a11 Taqman probe (Seq ID NO: 39) (5/6-FAM) TGT CGT TGT CAA TGC CGC CCA (5/6-TAMRA).

Taqman analysis of protein kinase, cAMP dependent regulatory, type II alpha (Prkar2a) was performed preferrably using the following primer/probe pairs: mouse Prkar2a forward primer (Seq ID NO: 40) 5′-GCC ACA AGA ACA CAC ACA GAA AA-3′; mouse Prkar2a reverse primer (Seq ID NO: 41) 5′-ATG GCA GCG GCG GAG-3′; mouse Prkar2a Taqman probe (Seq ID NO: 42) (5/6-FAM) AGA CAC GAC AGA ACC GCT CCT GCT G (5/6-TAMRA).

Taqman analysis of Cab45 was performed preferrably using the following primer/probe pairs: mouse Cab45 forward primer (Seq ID NO: 43) 5′-CGC AAC GTG CAT GAA GAG TT-3′; mouse Cab45 reverse primer (Seq ID NO: 44) 5′-AGA CAG GAC ACT GGC GAC CT-3′; mouse Cab45 Taqman probe (Seq ID NO: 45) (5/6-FAM) TGA GCA TGC CCC TCA GCT GGG (5/6-TAMRA).

Taqman analysis of the cell division protein FtsJ (FstJ) was performed preferrably using the following primer/probe pairs: mouse FstJ forward primer (Seq ID NO: 46) 5′-GCA GGA CTG AGG AAC AAG CC-3′; mouse FtsJ reverse primer (Seq ID NO: 47) 5′-CCT GGA GGC AGC AGC AAC-3′; mouse FtsJ Taqman probe (Seq ID NO: 48) (5/6-FAM) CAT CCA TAC CCC ACT CCC CGT CTC C (5/6-TAMRA).

Real time PCR (Taqman) analysis of the expression of spermidine synthase in mammalian (mouse) tissues revealed that spermidine synthase is expressed in a variety of adult mouse tissues including WAT and BAT. (FIG. 4A). The high experession levels of spermidine synthase in these tissues indicate, that spermidine synthase is involved in the metabolism of tissues relevant for the metabolic syndrome. The expression of spermidine synthase in brown adipose tissue is under metabolic control: In genetically obese (ob/ob) mice, expression is moderately induced compared to wildtype levels (FIG. 4B). In addition, expression of spermidine synthase is regulated during the in vitro differentiation of 3T3-L1 cells as well as of an additional model system for the in vitro differentiation of preadipocytes to adipocytes, the 3T3-F442A cell line (FIGS. 4C and 4D). Whereas spermidine synthase shows a transient upregulation of its expression in3T3-L1 cells, a stable upregulation of its expression in 3T3-F442A cells is noted. Spermidine synthase is upregulated by the end of the clonal expansion phase of preadipocyte differentaition. This suggests that spermidine synthase is neccssary during the onset of final adipocyte maturation (see FIGS. 4C and 4D).

Real time PCR (Taqman) analysis of the expression of RFamide-related peptide receptor and Gpr74 in mammalian (mouse) tissues revealed that both homologs, RFamide-related peptide receptor and Gpr74, demonstrate their highest expression in hypothalamus (FIGS. 8A and 8B). Overall, RFamide-related peptide receptor shows a higher expression level than Gpr74. Whereas Gpr74 is restricted in its expression to a few tissues, RFamide-related peptide receptor can—although at a quite low level—be detected in a variety of tissues including WAT and BAT. The expression of RFamide-related peptide receptor in liver ans spleen is under metabolic control: In fasted mice, expression is activated in these tissues compared to wildtype levels (FIG. 8C). The expression of Gpr74 in brown adipose tissue is under metabolic control: In genetically obese (ob/ob) mice, expression is moderately induced compared to wildtype levels (FIG. 8D). In addition, expression of RFamide-related peptide receptor is acvtivated during the in vitro differentiation of 3T3-L1 as well as of an additional model system for the in vitro differentiation of preadipocytes to adipocytes, the 3T3-F442A cell line (FIGS. 8E and 8F).

Real time PCR (Taqman) analysis revealed that Slc25a 1is ubiquitously expressed in adult mouse tissues showing highest expression in heart and skeletal muscle (FIG. 12A). As shown in FIG. 12B the expression in both tissues is about 2 fold lower in fasted animals. The expression and regulation of Slc25al 1 in both muscles tested is indicative of a potential role in metabolism.

Real time PCR (Taqman) analysis of the expression of PRKAR2A in mammalian (mouse) tissues revealed that PKAR2A is expressed in a variety of adult mouse tissues with higest levels in testis and muscle (FIG. 16A). The expresion of PRKAR2A is upregulated in WAT, BAT and the pancreas of genetically obese (FIG. 16B), genetically diabetic (FIG. 16C) and fasted (FIG. 16B) animals. These data suggest a metabolic control for PKAR2A in tissues involved in energy homeostasis. This hypothesis is further supported by the finding that PRKAR2A is transiently induced during preadipocyte differentiation (FIG. 16D).

Real time PCR (Taqman) analysis of the expression of Cab45 in mammalian (mouse) tissues revealed that Cab45 is expressed in a variety of adult mouse tissues including WAT and BAT (FIG. 20A). The high experession levels of Cab45 in these tissues indicate, that Cab45 is involved in the metabolism of tissues relevant for the metabolic syndrome. The expression of Cab45 is under metabolic control: In genetically obese ob/ob mice, the relative expression of Cab45 in BAT, liver, and pancreas is upregulated (FIG. 20B).

Real time PCR (Taqman) analysis of the expression of cell division protein FtsJ in mammalian (mouse) tissues revealed that cell division protein FtsJ is rather ubiquitously expressed (FIG. 24A). The expression of cell division protein FtsJ in brown adipose tissue is under metabolic control: In genetically obese (ob/ob) mice, expression is strongely induced compared to wildtype levels (FIG. 24B).

All publications and patents mentioned in the above specification are herein incorporated by reference.

Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A pharmaceutical composition comprising a nucleic acid molecule of the SRM, NPFF2, NPFF1, OX1R, OX2R, SLC25A11, PRKAR2A, PRKAR2B, Cab45, FtsJ2, AWP1, or ZNF216 gene family or a polypeptide encoded thereby or a fragment or a variant of said nucleic acid molecule or said polypeptide, or an effector, e.g. an antibody, an aptamer or another receptor recognizing a nucleic acid molecule of the SRM, NPFF2, NPFF1, OX1R, OX2R, SLC25A11, PRKAR2A, PRKAR2B, Cab45, FtsJ2, AWP1, or ZNF216 gene family or said polypeptide encoded thereby, preferably together with pharmaceutically acceptable carriers, diluents and/or adjuvants.

2. The composition of claim 1, wherein the nucleic acid molecule is a vertebrate or insect SRM, NPFF2, NPFF1, OX1 R, OX2R, SLC25A1 1, PRKAR2A, PRKAR2B, Cab45, FtsJ2, AWP1, or ZNF216 nucleic acid, particulary encoding Drosophila Gadfly Accession Number CG8327, human spermidine synthase (SRM; SEQ ID NO: 1; GenBank Accession Number NM_{—}003132), Drosophila Gadfly Accession Number CG10823, human neuropeptide FF 2 (NPFF2; SEQ ID NO: 4; GenBank Accession Number NM_{—}053036), human neuropeptide FF 1 (NPFF1; SEQ ID NO: 6; GenBank Accession Number NM_{—}022146), human orexin receptor 1 (OX 1 R; SEQ ID NO: 8; GenBank Accession Number NM_{—}001525), human orexin receptor 2 (OX2R; SEQ ID NO: 10; GenBank Accession Number NM_{—}01526), Drosophila Gadfly Accession Number CG18418, human solute carrier family 25 (mitochondrial carrier; oxoglutarate carrier) member 11 (SLC25A11; SEQ ID NO: 12; GenBank Accession Number NM_{—}03562), Drosophila Pka-R2 (Gadfly Accession Number CG15862), protein kinase, cAMP-dependent, regulatory, type II, alpha (PRKAR2A; SEQ ID NO: 14; GenBank Accession Number NM_{—}004157), human protein kinase, cAMP-dependent, regulatory, type II, beta (PRKAR2B; SEQ ID NO: 16; GenBank Accession Number NM-002736), Drosophila Gadfly Accession Number CG31475, human calcium binding protein Cab45 precursor variant 1 or 2 (Cab45; SEQ ID NO: 18 and 20; GenBank Accession Numbers NM_{—}16176 and NM_{—}16547), Drosophila Gadfly Accession Number CG 11447, human cell division protein FtsJ (FtsJ2; SEQ ID NO: 22; GenBank Accession Number NM_{—}013393), Drosophila Gadfly Accession Number CG 16751, human protein associated with PKR1 (AWP1; SEQ ID NO: 24; GenBank Accession Number NM_{—}1 9006), and/or human zinc finger protein 216 (ZNF216; SEQ ID NO: 26; GenBank Accession Number NM_{—}06007), or a fragment thereof or a variant thereof, and/or a sequence which is complementary thereto.

3. The composition of claim 1, wherein said nucleic acid molecule
(a) hybridizes at 50° C. in a solution containing 1×SSC and 0.1% SIDS to a nucleic acid molecule as defined in claim 2 or a nucleic acid molecule which is complementary thereto;
(b) it is degenerate with respect to the nucleic acid molecule of (a),
(c) encodes a polypeptide which is at least 85%, preferably at least 90%, more preferably at least 95%, more preferably at least 98% and up to 99.6% identical to a human spermidine synthase (SRM; SEQ ID NO: 2; GenBank Accession Number NP_{—}003123), human neuropeptide FF 2 (NPFF2; SEQ ID NO: 5; GenBank Accession Number NP_{—}444264), human neuropeptide FF 1 (NPFF1; SEQ ID NO: 7; GenBank Accession Number NP_{—}071429), human orexin receptor 1 (OX1 R; SEQ ID NO: 9; GenBank Accession Number NP_{—}001516), human orexin receptor 2 (OX2R; SEQ ID-NO: 11; GenBank Accession Number NP_{—}001517), human solute carrier family 25 mitochondrial carrier; oxoglutarate carrier) member 11 (SLC25A11; SEQ ID NO: 13; GenBank Accession Number NP_{—}003553), human protein kinase, cAMP-dependent, regulatory, type II, alpha (PRKAR2A; SEQ ID NO: 15; GenBank Accession Number NP_{—}004148), human protein kinase, cAMP-dependent, regulatory, type II, beta (PRKAR2B; SEQ ID NO: 17; GenBank Accession Number NP_{—}002727), human calcium binding protein Cab45 precursor variant 1 or 2 (Cab45; SEQ ID NO: 19 and 21; GenBank Accession Numbers NP_{—}057260 and NP_{—}057631), human cell division protein FtsJ (FtsJ2; SEQ ID NO: 23; GenBank Accession Number NP_{—}037525), human protein associated with PKR1 (AWP1; SEQ .ID NO: 25; GenBank Accession Number NP_{—}061879), and/or human zinc finger protein 216 (ZNF216; SEQ ID NO: 27; GenBank Accession Number NP_{—}005998);
(d) differs from the nucleic acid molecule of (a) to (c) by mutation and wherein said mutation causes an alteration, deletion, duplication or premature stop in the encoded polypeptide.

4. The composition of claim 1, wherein the nucleic acid molecule is a DNA molecule, particularly a cDNA or a genomic DNA.

5. The composition of claim 1, wherein said nucleic acid encodes a polypeptide contributing to regulating the energy homeostasis and/or the metabolism of triglycerides.

6. The composition of claim 1, wherein said nucleic acid molecule is a recombinant nucleic acid molecule.

7. The composition of claim 1, wherein the nucleic acid molecule is a vector, particularly an expression vector.

8. The composition of claim 1, wherein the polypeptide is a recombinant polypeptide.

9. The composition of claim 8, wherein said recombinant polypeptide is a fusion polypeptide.

10. The composition of claim 1, wherein said nucleic acid molecule is selected from hybridization probes, primers and anti-sense oligonucleotides.

11. The composition of claim 1 which is a diagnostic composition.

12. The composition of claim 1 which is a therapeutic composition.

13. The composition of claim 1 for the manufacture of an agent for detecting and/or verifying, for the treatment, alleviation and/or prevention of an disorders, including metabolic diseases such as obesity and other body-weight regulation disorders as well as related disorders such as metabolic syndrome, eating disorder, cachexia, diabetes mellitus, hypertension, coronary heart disease, hypercholesterolemia, dyslipidemia, osteoarthritis, gallstones and others, in cells, cell masses, organs and/or subjects.

14. Use of a nucleic acid molecule of SRM, NPFF2, NPFF1, OX1R, OX2R, SLC25A11, PRKAR2A, PRKAR2B, Cab45, FtsJ2, AWP1, or ZNF216 gene family or a polypeptide encoded thereby or a fragment or a variant of said nucleic acid molecule or said polypeptide or an effector, e.g. an antibody, an aptamer or another receptor recognizing a nucleic acid molecule of the SRM, NPFF2, NPFF1, OX1R, OX2R, SLC25A11, PRKAR2A, PRKAR213, Cab45, FtsJ2, AWP1, or ZNF216 gene family or a polypeptide encoded thereby for controlling the function of a gene and/or a gene product which is influenced and/or modified by a SRM, NPFF2, NPFF1, OX1R, OX2R, SLC25A11, PRKAR2A, PRKAR2B, Cab45, FtsJ2, AWP1, or ZNF216 homologous polypeptide.

15. Use of the nucleic acid molecule of the SRM, NPFF2, NPFF1, OX1 R, OX2R, SLC25A11, PRKAR2A, PRKAR2B, Cab45, FtsJ2, AWP1, or ZNF21 6 gene family or a polypeptide encoded thereby or a fragment or a variant of said nucleic acid molecule or said. polypeptide or an effector, e.g. antibody, an aptamer or another receptor recognizing a nucleic acid molecule of the SRM, NPFF2, NPFF1, OX1 R, OX2R, SLC25A11, PRKAR2A, PRKAR2B, Cab45, FtsJ2, AWP1, or ZNF216 gene family or a polypeptide encoded thereby for identifying substances capable of interacting with a SRM, NPFF2, NPFF1, OX1R, OX2R, SLC25A11, PRKAR2A, PRKAR2B, Cab45, FtsJ2, AWP1, or ZNF216 homologous polypeptide.

16. A non-human transgenic animal exhibiting a modified expression of a SRM, NPFF2, NPFF1, OX1R, OX2R, SLC25A11, PRKAR2A, PRKAR2B, Cab45, FtsJ2, AWP1, or ZNF216 homologous polypeptide.

17. The animal of claim 16, wherein the expression of the SRM, NPFF2, NPFF1, OX1 R, OX2R, SLC25A11, PRKAR2A, PRKAR2B, Cab45, FtsJ2, AWP1, or ZNF216 homologous polypeptide is increased and/or reduced.

18. A recombinant host cell exhibiting a modified expression of a SRM, NPFF2, NPFF1, OX1R, OX2R, SLC25A11, PRKAR2A, PRKAR2B, Cab45, FtsJ2, AWP1, or ZNF216 homologous polypeptide.

19. The cell of claim 18 which is a human cell.

20. A method of identifying a (poly)peptide involved in the regulation of energy homeostasis and/or metabolism of triglycerides in a mammal comprising the steps of
(a) contacting a collection of (poly)peptides with a SRM, NPFF2, NPFF1, OX1R, OX2R, SLC25A11, PRKAR2A, PRKAR2B, Cab45, FtsJ2, AWP1, or ZNF216 homologous polypeptide or a fragment thereof under conditions that allow binding of said (poly)peptides;
(b) removing (poly)peptides which do not bind and
(c) identifying (poly)peptides that bind to said SRM, NPFF2, NPFF1, 0X1 R, OX2R, SLC25A11, PRKAR2A, PRKAR2B, Cab45, FtsJ2, AWP1, or ZNF216 homologous polypeptide.

21. A method of screening for an agent which modulates the interaction of a SRM, NPFF2, NPFF1, OX1 R, OX2R, SLC25A1 1, PRKAR2A, PRKAR2B, Cab45, FtsJ2, AWP1, or ZNF216 homologous polypeptide with a binding targetagent, comprising the steps of
(a) incubating a mixture comprising
(aa)a SRM, NPFF2, NPFF1, OX1 R, OX2R, SLC25A11, PRKAR2A, PRKAR2B, Cab45, FtsJ2, AWP1, or ZNF216 homologous polypeptide, or a fragment thereof;
(ab) a binding target/agent of said SRM, NPFF2, NPFF1, OX1 R, MR, SLC25A11, PRKAR2A, PRKAR2B, Cab45, FtsJ2, AWP1, or ZNF216 homologous polypeptide or fragment thereof; and
(ac) a candidate agent under conditions whereby said SRM, NPFF2, NPFF1, OX1 R, MR, SLC25A11, PRKAR2A, PRKAR2B, Cab45, FtsJ2, AWP1, or ZNF216 polypeptide or fragment thereof specifically binds to said binding target/agent at a reference affinity;
(b) detecting the binding affinity of said SRM, NPFF2, NPFF1, OX1R, OX2R, SLC25A11, PRKAR2A, PRKAR2B, Cab45, FtsJ2, AWP1, or ZNF216 polypeptide or fragment thereof to said binding target to determine a (candidate) agent-biased affinity; and
(c) determining a difference between (candidate) agent-biased affinity and reference affinity.

22. A method for screening for an agent which modulates the activity of a SRM, NPFF2, NPFF1, OX1 R, OX2R, SLC25A11, PRKAR2A, PRKAR2B, Cab45, FtsJ2, AWP1, or ZNF216 homologous polypeptide, comprising the steps of
(a) incubating a mixture comprising
(aa) SRM, NPFF2, NPFF1, OX1 R, OX2R, SLC25A11, PRKAR2A, PRKAR2B, Cab45, FtsJ2, AWP1, or ZNF21 6 homologous polypeptide or a fragment thereof; and
(ab) a candidate agent under conditions whereby said SRM, NPFF2, NPFF1, OX1R, OX2R, SLC25A11, PRKAR2A, PRKAR2B, Cab45, FtsJ2, AWP1, or ZNF216 polypeptide or fragment thereof exhibits a reference activity;
(b) detecting the activity of said polypeptide or fragment thereof to determine a (candidate) agent-biased activity; and
(c) determining a difference between (candidate) agent-biased activity and reference activity.

23. A method of producing a composition comprising the (poly)peptide identified by the method of claim 20 with a pharmaceutically acceptable carrier, diluent and/or adjuvant.

24. The method of claim 23 wherein said composition is a pharmaceutical composition for preventing, alleviating or treating of diseases and disorders, including metabolic diseases such as obesity and other body-weight regulation disorders as well as related disorders such as metabolic syndrome, eating disorder, cachexia, diabetes mellitus, hypertension, coronary heart disease, hypercholesterolemia, dyslipidemia, osteoarthritis, gallstones, and other diseases and disorders.

25. Use of a (poly)peptide as identified by the method of claim 20 for the preparation of a pharmaceutical composition for the treatment, alleviation and/or prevention of diseases and disorders, including metabolic diseases such as obesity and other body-weight regulation disorders as well as related disorders such as metabolic syndrome, eating disorder, cachexia, diabetes mellitus, hypertension, coronary heart disease, hypercholesterolemia, dyslipidemia, osteoarthritis, gallstones, and other diseases and disorders.

26. Use of a nucleic acid molecule of the SRM, NPFF2, NPFF1, OX1 R, OX2R, SLC25A11, PRKAR2A, PRKAR2B, Cab45, FtsJ2, AWP1, or ZNF216 family or of a fragment thereof for the preparation of a non-human animal which over- or under-expresses the SRM, NPFF2, NPFF1, OX1R, OX2R, SLC25A11, PRKAR2A, PRKAR2B, Cab45, FtsJ2, AWP1, or ZNF216 gene product.

27. Kit comprising at least one of
(a) a SRM, NPFF2, NPFF1, OX1 R, OX2R, SLC25A1 1, PRKAR2A, PRKAR2B, Cab45, FtsJ2, AWP1, or ZNF216 nucleic acid molecule or a fragment thereof;
(b) a vector comprising the nucleic acid of (a);
(c) a host cell comprising the nucleic acid of (a) or the vector of (b);
(d) a polypeptide encoded by the nucleic acid of (a);
(e) a fusion polypeptide encoded by the nucleic acid of (a);
(f) an antibody, an aptamer or another receptor against the nucleic acid of (a) or the polypeptide of (d) or (e) and
(g) an anti-sense oligonucleotide of the nucleic acid of (a).

28. A method of producing a composition comprising mixing the (poly)peptide identified by the agent of claim 21 with a pharmaceutically acceptable carrier, diluent and/or adjuvant.

29. Use of a (poly)peptide agent as identified by the method of claim 21 for the preparation of a pharmaceutical composition for the treatment, alleviation and/or prevention of diseases and disorders, including metabolic diseases such as obesity and other body-weight regulation disorders as well as related disorders such as metabolic syndrome, eating disorder, cachexia, diabetes mellitus, hypertension, coronary heart disease, hypercholesterolemia, dyslipidemia, osteoarthritis, gallstones, and other diseases and disorders.
